SEQUENCE LISTING (20 sequences)

SEQ ID NO: 1 - DNA, 1085 bp, Mus musculus
  source 1..1085: mol_type = other DNA
cttcctttctttgtagccacctcaggggaagcaacagatcgtcactcggtgttctcaccgaaagcacgtaatcgccggtgtaactcatgttggctggggggcctccccgcgcgcagaaaggctggggtgcgcccccgggcagctctcctttgctcagctacatggtcctggtccacgagtgctctgagggcggcaagagagcgcaactcctgacgcctccccccactccccggggttcggagtgactcctcagagccagaggcacttctgctcaccggtccgcaagctgcctggtctgctgaagctgacgaatcgggaaaccatgcaattgaggcgaaccttgggctgttttagaggcgctgaggagccttctcctgggaggcccaaggttgatttcagcccaccaggatctggggaagacccaactagggataagagcacaccaaaaggccaagtccgagttccatttctagaagaggcggcttccggcaaggctatgacattggccctggacattggtttcccaggagctgctttttctcaagaactccacagcacggggctgtctccagaaaacgctcttcaacgtttatttcttttaatcgtcgcccggagccctaaggcggctaatgcaagaggccaaaaatgtttggaggaagaaaaacaaaggcaggaagtggccgcggcctgacggtgcgtgtgtgtctgtaaagaagggagggagccggttcaacctcccctcgttttcccgaacttcaaggtctaggcagacccccttagggccttgccgaggctcgcccccacacccccagcggcgcagcatttggaggtggccaacgatttaagcctcggtcgggctgaaaggagatttgatcggcagaacaaaccaacccttttcggaggtttcttttgatttggtcctaaagggtatatgctagtgtccacagcggctggggtggctgctgttttcctcccgccgggctaaaagtaccaagaagggagggagggagagagattcaggcaccttgcgctggctgcactctccttctgagatagaataccagaataaagtgtattcaggtgcc

SEQ ID NO: 2 - DNA, 1552 bp, Mus musculus
  source 1..1552: mol_type = other DNA
cttcctttctttgtagccacctcaggggaagcaacagatcgtcactcggtgttctcaccgaaagcacgtaatcgccggtgtaactcatgttggctggggggcctccccgcgcgcagaaaggctggggtgcgcccccgggcagctctcctttgctcagctacatggtcctggtccacgagtgctctgagggcggcaagagagcgcaactcctgacgcctccccccactccccggtgggtgagggatactctctgggctgggggtggccaggtgaaagcccggaattgtatagcttcaggtcccggagtctgttatccgaaggcttacgttcagcaccttcttcgcagcccccctcccacaggtttgctctgggaagccccccagcctcagaccctggctggaccccatttggggccaggcttcgccggcacggatgtgccggcctcgtggcttgtccgatttgcacggtgacttgattacacgctctcattcacggtcacttccgaagcgctttagtgccttccgtccccaaaccgccaacaggcaaagcggcttccctccgcggtttgtcaataatccgcgctgtccggaagggccttcgtcttacccgggttccaccttccctgtatctttctgcttacttccttaccccacaccctgtccttgaaggaaccccttctcctcgctgcctgtaggggttcggagtgactcctcagagccagaggcacttctgctcaccggtccgcaagctgcctggtctgctgaagctgacgaatcgggaaaccatgcaattgaggcgaaccttgggctgttttagaggcgctgaggagccttctcctgggaggcccaaggttgatttcagcccaccaggatctggggaagacccaactagggataagagcacaccaaaaggccaagtccgagttccatttctagaagaggcggcttccggcaaggctatgacattggccctggacattggtttcccaggagctgctttttctcaagaactccacagcacggggctgtctccagaaaacgctcttcaacgtttatttcttttaatcgtcgcccggagccctaaggcggctaatgcaagaggccaaaaatgtttggaggaagaaaaacaaaggcaggaagtggccgcggcctgacggtgcgtgtgtgtctgtaaagaagggagggagccggttcaacctcccctcgttttcccgaacttcaaggtctaggcagacccccttagggccttgccgaggctcgcccccacacccccagcggcgcagcatttggaggtggccaacgatttaagcctcggtcgggctgaaaggagatttgatcggcagaacaaaccaacccttttcggaggtttcttttgatttggtcctaaagggtatatgctagtgtccacagcggctggggtggctgctgttttcctcccgccgggctaaaagtaccaagaagggagggagggagagagattcaggcaccttgcgctggctgcactctccttctgagatagaataccagaataaagtgtattcaggtgcctctgc

SEQ ID NO: 3 - DNA, 2126 bp, Mus musculus
  source 1..2126: mol_type = other DNA
atttaaaaacaattgtgaataacagaaggcagcatgtaattgcaaccggaccagaggaaaatggcttcctttggacaaaaaggccaaatttaagttaatttgtttcttttaatatattgcttgagctaagtgggctactttatctgttaaatgcgctcttagcgccatcattaaacagccgatgcctttgaatcccagccgggactggagtcccgccgcaacacatggcttttataaaaatctccggattaccccgctatcaaaggctcctgaagcccttgtagggggaatttacaggcgcccacctccggctccccagcctggagctggtctctagcggggatcgtgctgttgagtttgggagctgagaagggaaaaaaggaaaaaggggagttgtatttttttttttttctgcaggcatgctttggccggtgggtatttcacggccaatttcagcactcgccacgtgatccctccttttataacaaagttttgttgggggaaacctaaaggcccttcataaaccttatatgcttataaaacagcatataaaaatttaacagcggtgctgcgctagattcccaactcccctttcataaagcgctgggcgctgcctttatacgcaccggagccaccggccttgtcctcagtgtggccggaagacgactcggggctgctgctgctctcgggtcgccgccgtcgctccttctcctccgccgccgcctcctgacagccgccgccaccgccgccgtccgaacttgaagttgccggcgcgcccgtggccgccgccgccaccgccgcagcggaggtcgcagctgctgcttggggccccttctcggcgttcttgtccccggggtagtcggaggaagcgaggttttccggggtgccgtaagccgtctcgaaaaactggtcgaaagcctgtggcagaacgccgttcctgcccaccgtgctatagaaattggacgagacggcgggggtggggtggtggtagacgttggccgagctcttggccagcacgtcgccaggcacgccggccgcgctgggcgcctgcagacagtctctgtgcacgagctcctccgcggagtagcagtgggccagattgccgcgggggtgccatttagtggcgggctcaatggcgtactctctgaaggtcacttcgcgcacgggttggacctggggcaggttggaggagtaggagtatgtcattgggcgcgaagacggggtctggggcaaaaaagaagggaggctggagaaatctggacccgagacgtagggttcggagtgactcctcagagccagaggcacttctgctcaccggtccgcaagctgcctggtctgctgaagctgacgaatcgggaaaccatgcaattgaggcgaaccttgggctgttttagaggcgctgaggagccttctcctgggaggcccaaggttgatttcagcccaccaggatctggggaagacccaactagggataagagcacaccaaaaggccaagtccgagttccatttctagaagaggcggcttccggcaaggctatgacattggccctggacattggtttcccaggagctgctttttctcaagaactccacagcacggggctgtctccagaaaacgctcttcaacgtttatttcttttaatcgtcgcccggagccctaaggcggctaatgcaagaggccaaaaatgtttggaggaagaaaaacaaaggcaggaagtggccgcggcctgacggtgcgtgtgtgtctgtaaagaagggagggagccggttcaacctcccctcgttttcccgaacttcaaggtctaggcagacccccttagggccttgccgaggctcgcccccacacccccagcggcgcagcatttggaggtggccaacgatttaagcctcggtcgggctgaaaggagatttgatcggcagaacaaaccaacccttttcggaggtttcttttgatttggtcctaaagggtatatgctagtgtccacagcggctggggtggctgctgttttcctcccgccgggctaaaagtaccaagaagggagggagggagagagattcaggcaccttgcgctggctgcactctccttctgagatagaataccagaataaagtgtattcaggtgcct

SEQ ID NO: 4 - DNA, 1116 bp, Mus musculus
  source 1..1116: mol_type = other DNA
cttcctttctttgtagccacctcaggggaagcaacagatcgtcactcggtgttctcaccgaaagcacgtaatcgccggtgtaactcatgttggctggggggcctccccgcgcgcagaaaggctggggtgcgcccccgggcagctctcctttgctcagctacatggtcctggtccacgagtgctctgagggcggcaagagagcgcaactcctgacgcctccccccactccccggaaccccttctcctcgctgcctgtaggggttcggagtgactcctcagagccagaggcacttctgctcaccggtccgcaagctgcctggtctgctgaagctgacgaatcgggaaaccatgcaattgaggcgaaccttgggctgttttagaggcgctgaggagccttctcctgggaggcccaaggttgatttcagcccaccaggatctggggaagacccaactagggataagagcacaccaaaaggccaagtccgagttccatttctagaagaggcggcttccggcaaggctatgacattggccctggacattggtttcccaggagctgctttttctcaagaactccacagcacggggctgtctccagaaaacgctcttcaacgtttatttcttttaatcgtcgcccggagccctaaggcggctaatgcaagaggccaaaaatgtttggaggaagaaaaacaaaggcaggaagtggccgcggcctgacggtgcgtgtgtgtctgtaaagaagggagggagccggttcaacctcccctcgttttcccgaacttcaaggtctaggcagacccccttagggccttgccgaggctcgcccccacacccccagcggcgcagcatttggaggtggccaacgatttaagcctcggtcgggctgaaaggagatttgatcggcagaacaaaccaacccttttcggaggtttcttttgatttggtcctaaagggtatatgctagtgtccacagcggctggggtggctgctgttttcctcccgccgggctaaaagtaccaagaagggagggagggagagagattcaggcaccttgcgctggctgcactctccttctgagatagaataccagaataaagtgtattcaggtgcctctgc

SEQ ID NO: 5 - DNA, 1549 bp, Homo sapiens
  source 1..1549: mol_type = other DNA
gccacctcaggggaagcaacagatcgtcactcggtgttctcaccgaaagcacgtaatcgccggtgtaactcatgttggctggggggcctcccggcgcgcgcggagaggctggggtgcgcccccatgcagcatgcttgtgctcaattgcagggtcctcgttctcgagtgtgcagagggcggtgagagctcaactctcgtccccacctcccacccgcagctccccgggtgggtgagggatgccctggactggggatagccaggtgggagtccgtcgctgtgtggcctgtggtctcggagtctgttctcctggagtctcgcatttgcacccccttcttcgcagtccccctcccatagacttgctctgggaagcgcctctgcctccgaccctagccggaaccccttcggggccagagtttgaagccgtggatgtgcctgcctggtggcttgtccgatttgcacggtgacttgattacactctctcattcatggtcacttccgaagcgctttagtgccttccgtccctaaaccgccaacagccagaacggcttctccccgcggtttgtcactgatccgcagggcccggaagggccttcgtcttacccgggatccacctctcccctcatcttccctgcctacctcttcatcccaccttctgtccttggagaaactccctcctcctcgctgcctgccgggcttcggagtgactcggcagagacagaggcacaggggctgccctgctgctcaccggtccacccatctgcctggtcttctggagctgaggactcgggaaaccatgcaattgaggcaagccttgggctgctttagaggcgctgacatccgaggagacttctcctgggaggtccaacagccgagcttagcccaccgggctctgggaaagacccgactgaggctaaagccgccccggaaggccaagtccgagttccatttcttgaagaggccggcgcgcgtaaggctgtgacattggccctggcgactggcttcccaggagctgttctttctcaggagctccacagcgcgggccatctccagaaaactgtcttcagagtgtatttccttttatcgtcaacccagagccccaccgcggctaatgcaagaggccaaaaaatgtttggaggaagaaaaacaaaggcaggaagtggcggcggcctgacggtgcgtgtgtgtctgcagagaagggagggagccggctcagtctcttcttgtttttccaaacttcaaggtccaggcagccctctgcagggccgggccccattgctccccgcgcggcattggaggtggccgcccggagaggagaaggccaacgcctgcgccaggcttgtcaggcggaaacggctaacaaggagatttggtcagcaaaacagacccagcctttccgaggcttcgtctgacttggcccgaaaggttggggagggggggcttgcgcagagcctcagggaccctcctctctggggactaccatccctgagccttacgcttctttccacagcctttgcaggcggaatatcggaataaagtgggtccaggcgcc

SEQ ID NO: 6 - DNA, 1201 bp, Homo sapiens
  source 1..1201: mol_type = other DNA
cagcatgcttgtgctcaattgcagggtcctcgttctcgagtgtgcagagggcggtgagagctcaactctcgtccccacctcccacccgcagctccccgggtgggtgagggatgccctggactggggatagccaggtgggagtccgtcgctgtgtggcctgtggtctcggagtctgttctcctggagtctcgcatttgcacccccttcttcgcagtccccctcccatagacttgctctgggaagcgcctctgcctccgaccctagccggaaccccttcggggccagagtttgaagccgtggatgtgcctgcctggtggcttgtccgatttgcacggtgacttgattacactctctcattcatggtcacttccgaagcgctttagtgccttccgtccctaaaccgccaacagccagaacggcttctccccgcggtttgtcactgatccgcagggcccggaagggccttcgtcttacccgggatccacctctcccctcatcttccctgcctacctcttcatcccaccttctgtccttggagaaactccctcctcctcgctgcctgccgggcttcggagtgactcggcagagacagaggcacaggggctgccctgctgctcaccggtccacccatctgcctggtcttctggagctgaggactcgggaaaccatgcaattgaggcaagccttgggctgctttagaggcgctgacatccgaggagacttctcctgggattgaaggagcaatgtttggaggaagcgaaagaaaggagaggacacagagcacagagcagccaggcagagccaggagctgagaagggcccagacctgaggcctcccaacaactctcttcttggaaggatctgggatgttgctgaaggaaaataaaaaaatatgtaaaaagataaccttttgtttttccctctccaggaaatagccaaagttatttacatatcttggggagatttagagtataaactctaagatctttggtatttaagtgtcaacatcgatttatttatttattgctgagctgactgtaactgactcaataacaaatctaatcgtgtattgcactggaaaagaaatattcttattatgtattttctccaaataatggcctaccattgcatttgaatacctgctgtaaatatcaataatatgaagtaattactctgtagtcgagtaaactaatttattagca

SEQ ID NO: 7 - DNA, 369 bp, synthetic construct
  source 1..369: mol_type = other DNA
atgcaattgaggcgaaccttgggctgttttagaggcgctgaggagccttctcctgggaggcccaaggttgatttcagcccaccaggatctggggaagacccaactagggataagagcacaccaaaaggccaagtccgagttccatttctagaagaggcggcttccggcaaggctatgacattggccctggacattggtttcccaggagctgctttttctcaagaactccacagcacggggctgtctccagaaaacgctcttcaacgtttatttcttttaatcgtcgcccggagccctaaggcggctaatgcaagaggccaaaaatgtttggaggaagaaaaacaaaggcaggaagtggccgcggcctga

SEQ ID NO: 8 - DNA, 216 bp, synthetic construct
  source 1..216: mol_type = other DNA
atgcaagaggccaaaaatgtttggaggaagaaaaacaaaggcaggaagtggccgcggcctgacggtgcgtgtgtgtctgtaaagaagggagggagccggttcaacctcccctcgttttcccgaacttcaaggtctaggcagacccccttagggccttgccgaggctcgcccccacacccccagcggcgcagcatttggaggtggccaacgatttaa

SEQ ID NO: 9 - DNA, 159 bp, synthetic construct
  source 1..159: mol_type = other DNA
atgttggctggggggcctccccgcgcgcagaaaggctggggtgcgcccccgggcagctctcctttgctcagctacatggtcctggtccacgagtgctctgagggcggcaagagagcgcaactcctgacgcctccccccactccccggggttcggagtga

SEQ ID NO: 10 - DNA, 384 bp, synthetic construct
  source 1..384: mol_type = other DNA
atgttggctggggggcctccccgcgcgcagaaaggctggggtgcgcccccgggcagctctcctttgctcagctacatggtcctggtccacgagtgctctgagggcggcaagagagcgcaactcctgacgcctccccccactccccggtgggtgagggatactctctgggctgggggtggccaggtgaaagcccggaattgtatagcttcaggtcccggagtctgttatccgaaggcttacgttcagcaccttcttcgcagcccccctcccacaggtttgctctgggaagccccccagcctcagaccctggctggaccccatttggggccaggcttcgccggcacggatgtgccggcctcgtggcttgtccgatttgcacggtga

SEQ ID NO: 11 - DNA, 234 bp, synthetic construct
  source 1..234: mol_type = other DNA
atgtgccggcctcgtggcttgtccgatttgcacggtgacttgattacacgctctcattcacggtcacttccgaagcgctttagtgccttccgtccccaaaccgccaacaggcaaagcggcttccctccgcggtttgtcaataatccgcgctgtccggaagggccttcgtcttacccgggttccaccttccctgtatctttctgcttacttccttaccccacaccctgtccttga

SEQ ID NO: 12 - DNA, 76 bp, synthetic construct
  source 1..76: mol_type = other DNA
cacgagtgctctgaaataaaagatctttattttcattagatctgtgtgttggttttttgtgtggggcggcaagaga

SEQ ID NO: 13 - DNA, 22 bp, synthetic construct
  source 1..22: mol_type = other DNA
gctcagctacatggtcctggtc

SEQ ID NO: 14 - DNA, 21 bp, synthetic construct
  source 1..21: mol_type = other DNA
tccgggacctgaagctataca

SEQ ID NO: 15 - DNA, 21 bp, synthetic construct
  source 1..21: mol_type = other DNA
cattagatctgtgtgttggtt

SEQ ID NO: 16 - DNA, 24 bp, synthetic construct
  source 1..24: mol_type = other DNA
gcagctctcctttgctcagctaca

SEQ ID NO: 17 - DNA, 17 bp, synthetic construct
  source 1..17: mol_type = other DNA
ctctcttgccgccctca

SEQ ID NO: 18 - DNA, 33 bp, synthetic construct
  source 1..33: mol_type = other DNA
agatcggaagagcacacgtctgaactccagtca

SEQ ID NO: 19 - DNA, 4060 bp, Homo sapiens
  source 1..4060: mol_type = other DNA
acgtaagctgacgtggaaatctatccccatccttagcagggaggtgctggtcatgtgacccgatgttgaaattgacaagctgctagctagtccgggccttttccccccccctttccttttttttttttcctcctctcccctccctcccggcttcctttctttgtagccacctcaggggaagcaacagatcgtcactcggtgttctcaccgaaagcacgtaatcgccggtgtaactcatgttggctggggggcctcccggcgcgcgcggagaggctggggtgcgcccccatgcagcatgcttgtgctcaattgcagggtcctcgttctcgagtgtgcagagggcggtgagagctcaactctcgtccccacctcccacccgcagctccccgggtgggtgagggatgccctggactggggatagccaggtgggagtccgtcgctgtgtggcctgtggtctcggagtctgttctcctggagtctcgcatttgcacccccttcttcgcagtccccctcccatagacttgctctgggaagcgcctctgcctccgaccctagccggaaccccttcggggccagagtttgaagccgtggatgtgcctgcctggtggcttgtccgatttgcacggtgacttgattacactctctcattcatggtcacttccgaagcgctttagtgccttccgtccctaaaccgccaacagccagaacggcttctccccgcggtttgtcactgatccgcagggcccggaagggccttcgtcttacccgggatccacctctcccctcatcttccctgcctacctcttcatcccaccttctgtccttggagaaactccctcctcctcgctgcctgccgggcttcggagtgactcggcagagacagaggcacaggggctgccctgctgctcaccggtccacccatctgcctggtcttctggagctgaggactcgggaaaccatgcaattgaggcaagccttgggctgctttagaggcgctgacatccgaggagacttctcctgggtatgctgcatcttcgtggggggcccatcaggctgcttacagagctccaggggtgtggggagaatggaggtggaaaggacgggctgagggccaaggagaggtggctgagaaaggggtaacccacctactctcctactctccttccttgcaatgtgtgagagtgaaagcaagggatgtgcagggcaaaaactcagagagctctgtgcctttcctactgctcattccacaggaggaatacaataccagagaaaggagaggtgcttcccgaactccctgcacctggggaaacagggatgtctttaaaaagctaggtttgtgctaaggatagagagaggccatagcaatactctgagtctagctttcttgagaagaggaaaaaaaataaattaagaaggcaaaatatgctccccatcctgcaggattgaaggagcaatgtttggaggaagcgaaagaaaggagaggacacagagcacagagcagccaggcagagccaggagctgagaagggcccagacctgaggcctcccaacaactctcttcttggaaggatctgggatgttgctgaaggaaaataaaaaaatatgtaaaaagataaccttttgtttttccctctccaggaaatagccaaagttatttacatatcttggggagatttagagtataaactctaagatctttggtatttaagtgtcaacatcgatttatttatttattgctgagctgactgtaactgactcaataacaaatctaatcgtgtattgcactggaaaagaaatattcttattatgtattttctccaaataatggcctaccattgcatttgaatacctgctgtaaatatcaataatatgaagtaattactctgtagtcgagtaaactaatttattagcattaatgtttatgtggctctccacctccccccaccaccacctttacaaggattgcttatcacaaatcaagctacttggacacattggtttaatgaactctttattcaagatttgctacaagaattttcatgtcctttgaatccctgagaactgaacttgaaattatttgtgcatcttcagcttgacatatttgtccactgtggcttgtccagagggcagcagtgctgtgagtgagaaggtccagtgggaaggaaggttattggagaagagtagcctcagacctcctaaagctgggaagcacatttacagaattgccctgcagcgaaaaaacttctatttccagcagtgaccaacaaggcaaaatgtttgtttctccacagcatctcttttttagagacagaatataaaagacagaaggagaggtttcaaacagcggtattcgagctgttccctggcttgattttggctatcccaagctctctctctgcagcccacccagtccacgccacccctaccttcgaacaaaaggaatgcatgaagggtttcagtgactttgccataacaaaggcgccaccattgcggggctcgccccgcccctgggtgaaggcaaacaaattcttgcacttgtattagggcttttaagaccataattgaacccgggggcgtctaggaaaaccgaaaacagttctagacagacctgcggttttatagcagttttggcagtcaacttcagcttgtgcctgagcagacggggttgtggtggcccgccagcgggggatgccaggccacctcccccagcggcacgcagcccctctcttaattagatcggttttcccctggtgtccgggagagcggtcccggcagaaaggtcggtatgggggtgtgcgctgttccgcataaccactgcctcccatgtcctcctcgagggccgaaccgagagggtgctggcagggctggatcccacgggtgtccgcaggagacaaaggcgaattccggagaaaaggctggggctgagaaaggcgctccgggagcggctggcagggcaattcggcaggctgcaccgaagccgagtgcccggagggacttgccgcccggaagggggtgtgtgggggcgctgccgtgaagatggatgagggaaaaggtttttgatatcagcagaagggaaaacgcctggagtggccgaacacttttagttgcccagcaggaataggagacgggtactcagctccccaaggctgcgcaatatcccagctttgcccgctcctgccctcgtgttcggaatatgctggcggtgtgaatgtgaaggtttctccaggcaggcggctggggcgaggggtgggcgcagctctgaggatcagaccaaatctaggggaaaaaggggaggcagaccttcgggtccttggttttgactttgctctgagcctatgattgactcttccgctttgcaggaggtccaacagccgagcttagcccaccgggctctgggaaagacccgactgaggctaaagccgccccggaaggccaagtccgagttccatttcttgaagaggccggcgcgcgtaaggctgtgacattggccctggcgactggcttcccaggagctgttctttctcaggagctccacagcgcgggccatctccagaaaactgtcttcagagtgtatttccttttatcgtcaacccagagccccaccgcggctaatgcaagaggccaaaaaatgtttggaggaagaaaaacaaaggcaggaagtggcggcggcctgacggtgcgtgtgtgtctgcagagaagggagggagccggctcagtctcttcttgtttttccaaacttcaaggtccaggcagccctctgcagggccgggccccattgctccccgcgcggcattggaggtggccgcccggagaggagaaggccaacgcctgcgccaggcttgtcaggcggaaacggctaacaaggagatttggtcagcaaaacagacccagcctttccgaggcttcgtctgacttggcccgaaaggttggggagggggggcttgcgcagagcctcagggaccctcctctctggggactaccatccctgagccttacgcttctttccacagcctttgcaggcggaatatcggaataaagtgggtccaggcgcctctgccgcc

SEQ ID NO: 20 - DNA, 3985 bp, Mus musculus
  source 1..3985: mol_type = other DNA
acgtaagctgacgtggaaatctatccccatccttagcagggaggtgctggtcatgtgacccgatgttgaaattgacaagccgcgagctagtcccggcttttttttttttttaacccccctccctttccttttttcccctcccctccctcctcggcttcctttctttgtagccacctcaggggaagcaacagatcgtcactcggtgttctcaccgaaagcacgtaatcgccggtgtaactcatgttggctggggggcctccccgcgcgcagaaaggctggggtgcgcccccgggcagctctcctttgctcagctacatggtcctggtccacgagtgctctgagggcggcaagagagcgcaactcctgacgcctccccccactccccggtgggtgagggatactctctgggctgggggtggccaggtgaaagcccggaattgtatagcttcaggtcccggagtctgttatccgaaggcttacgttcagcaccttcttcgcagcccccctcccacaggtttgctctgggaagccccccagcctcagaccctggctggaccccatttggggccaggcttcgccggcacggatgtgccggcctcgtggcttgtccgatttgcacggtgacttgattacacgctctcattcacggtcacttccgaagcgctttagtgccttccgtccccaaaccgccaacaggcaaagcggcttccctccgcggtttgtcaataatccgcgctgtccggaagggccttcgtcttacccgggttccaccttccctgtatctttctgcttacttccttaccccacaccctgtccttgaaggaaccccttctcctcgctgcctgtaggggttcggagtgactcctcagagccagaggcacttctgctcaccggtccgcaagctgcctggtctgctgaagctgacgaatcgggaaaccatgcaattgaggcgaaccttgggctgttttagaggcgctgaggagccttctcctgggtatgctgcaccttggggtgggggtgggggggggcaccaggctgcttgcagggcttcagagctgtgagggaaaaggagggtggcaaagagggagtactggcccaaggagtcctggctaagaaaagagtctcttctttcttgcaaaacctaagagtgaaagcaaaggatattcaaggcagaaattgagacagctcccatcattcttgttctttagcccacaggagaagcacaatgccaaagaaaagagagatgctctcctgcctgcctgcaccttgggccacagagatgcctcaaaagggcaaggcttgctgtacagatagataattctaaagaggccttggctgctaagttgcatttagctttctcccgaggaggaaatggggattaagaagataaaataacgactgccattctgcaagaccaaagatgtacttaggggaaaatgaaagagaaaaaagagggcgcaggacagctaggcaaattgagcagagatggggctccagaccaggggtctcaccacaactcccttcttggaaggtgctagggtgtcacaacagtcaaaagtgaaaaatgaaaacctttttccctttcctcattcaggaaatagttaaaggtatttacatatcttggggagatttagagtataaacgctaagatctttggtatttaagtgtcaacatcgatttatttatttattgctgagctgactgtaactgactcaataacaaatttaatcatgtattgctctggaaaagaactactctcgttatgtattttctccaaataatgacccaccattgcatatgaatgcccactgtaaatatcaataatatcaagtaattactgtgtagtggagtaaactaatttattagcattaatgtttatgtggctccaacccccctctcttttacaagggctgcttatcacaaatcaagctactcggacacattggtttaatgaactctttattcaggatttgctgcaggaactttcatgccccttgaaatccctgagagctgaacttgaaattatttgtgcatcttcagcttgacatatttgttcactgtggcttcatagatgactgccacggcgtttgcatgagattcaggagggagagagggtgttgggaaagggtagcttcaggtcttctaaagctgaggatcacaacgacaggatggtcctgcagcaaaaacttctatttctatcagttaccaaggaggcaagcatatctgtctcttcacggttgtctttctggagatccggatagaaaagggaggtgggcttgcaaaaggccttacaagaacagctctctgggttggttttgctacctcaaactttcttcagcagcccacccagtcctcaataccccttccctcgaacaaaaggaatgcatgaagggtttcagtgactttgctataacaaaggcgccaccattgcagggctcgccccgcccctgggtgaaggcaaacaaattcttgcacttgcattagggcttttaagaccataattgaacccgggggcgtctaggagaaccgaaaacagttctagacagacctgtggttttatagcagttttggcagtcaacttcagcttgtgcctgagcagtggggcccgaggtggctcagccgcgggggacgccaggccgcctcccctgactacatggcagcttctctcataattaactcgacttcccgggatgccagggagaaggggtacccacagaaaggtcggtgcggcactgtgcagtgttctgcacagccagtgcttcctaggttctcggggagggctaattgaataggagccggcaggtctgtatcccatgggtgtcagcaggagacaagacaaattcccgagcaaagactgcggaagaagaaggagccccacagaggctgacagagcaattcgacaggctctacagaagaggggtgcccagaggggcatcctcgaagggagcgtgtgggggcgctgcgatgaagatggatgagggaaaggcttttgatgggggtgggggggaggcggatggcctggagatgccgattgcgtttagttctcttaagcagagagaagaaatggatgctcagatccctaaggctgctcaatagtagccgctttgcctgcttggccctggtagtgtgaatgttcttcagtcatgcatcagggtacaaggtgcgagcagccctgaggttcggaccaaatctggaggaagaaagggaggtctctgagggtccttggttttcactttgctttgagcttaactggttctttggctttccaggaggcccaaggttgatttcagcccaccaggatctggggaagacccaactagggataagagcacaccaaaaggccaagtccgagttccatttctagaagaggcggcttccggcaaggctatgacattggccctggacattggtttcccaggagctgctttttctcaagaactccacagcacggggctgtctccagaaaacgctcttcaacgtttatttcttttaatcgtcgcccggagccctaaggcggctaatgcaagaggccaaaaatgtttggaggaagaaaaacaaaggcaggaagtggccgcggcctgacggtgcgtgtgtgtctgtaaagaagggagggagccggttcaacctcccctcgttttcccgaacttcaaggtctaggcagacccccttagggccttgccgaggctcgcccccacacccccagcggcgcagcatttggaggtggccaacgatttaagcctcggtcgggctgaaaggagatttgatcggcagaacaaaccaacccttttcggaggtttcttttgatttggtcctaaagggtatatgctagtgtccacagcggctggggtggctgctgttttcctcccgccgggctaaaagtaccaagaagggagggagggagagagattcaggcaccttgcgctggctgcactctccttctgagatagaataccagaataaagtgtattcaggtgcctctgctgaa

(19) United States
(12) Patent Application Publication
(10) Pub. No.: US 2026/0218299 A1
(43) Pub. Date: Jul. 30, 2026

(54) HOXA11AS LONG NON-CODING RNA IN INFLAMMATORY BOWEL DISEASE

(71) Applicant: UNIVERSITY OF MASSACHUSETTS, Westborough, MA (US)

(72) Inventors: Katherine A. FITZGERALD, Westborough, MA (US); Liraz GALIA, Westborough, MA (US)

(21) Appl. No.: 19/143,050

(22) PCT Filed: Jul. 25, 2024
(86) PCT No.: PCT/US2024/039483

§ 371 Date: Jun. 25, 2025

Related U.S. Application Data
(60) Provisional application No. 63/528,833, filed on Jul. 25, 2023.
(60) Provisional application No. 63/532,249, filed on Aug. 11, 2023.

(51) Int. Cl. C12Q 1/6883; A61K 31/7105; C12N 15/113; C12N 15/86; C12N 15/88; C12Q 1/6809; C12Q 1/686

(52) U.S. Cl. CPC C12Q 1/6883; A61K 31/7105; C12N 15/113; C12N 15/86; C12N 15/88; C12Q 1/6809; C12Q 1/686; C12N 2710/10043; C12N 2740/15043; C12N 2750/14143; C12Q 2600/106

(57) ABSTRACT

Provided herein are gene replacement approaches to restore HOXA11AS in the colon for patients with IBD, e.g., UC. Further, since HOXA11os levels inversely correlate with disease severity this lncRNA can also be used as a sensitive colon specific disease relevant biomarker.

### CLAIM OF PRIORITY

This application claims the benefit of U.S. Provisional Patent Application Ser. No. 63/528,833, filed on Jul. 25, 2023, and 63/532,249, filed on Aug. 11, 2023. The entire contents of the foregoing are hereby incorporated by reference herein.

### SEQUENCE LISTING

This application contains a Sequence Listing that has been submitted electronically as an XML file named “07917-0448WO1.xml” The XML file, created on Jul. 24, 2024, is 36,864 bytes in size. The material in the XML file is hereby incorporated by reference in its entirety.

### TECHNICAL FIELD

Provided herein are gene replacement approaches to restore HOXA11AS in the colon for patients with inflammatory bowel disease (IBD), e.g., ulcerative colitis (UC). Further, since HOXA11os levels inversely correlate with disease severity methods are provided for using this lncRNA as a sensitive colon specific disease relevant biomarker.

### BACKGROUND

Ulcerative colitis (UC) is a chronic inflammatory disease of the colon tissue. UC is a multifactorial disease with underlying causes attributed to dysregulated immune responses, defects in epithelial barrier integrity as well as genetic and environmental factors. To date the majority of UC related studies have focused on delineating the functional roles proteins play in the development of the disease. Although significant progress has been made in understanding the disease etiology, current UC treatments only alleviate symptoms and for many individuals fail to maintain long term remission of the disease.

### SUMMARY

In this study, we have identified a previously unannotated disease relevant transcript of a lncRNA in the HOX locus, termed HOXA11os. HOXA11os is uniquely expressed in the distal colon in both mice and humans where its expression inversely correlates with inflammation and disease severity. Moreover, mice lacking HOXA11os are highly susceptible to colitis. HOXA11os is localized to the mitochondria under basal conditions where it interacts with complex I of the Electron Transfer Chain (ETC) that feeds into mitochondrial oxidative phosphorylation (OXPHOS). Mitochondria in HOXA11os deficient colonic myeloid cells are dysfunctional characterized by reduced levels of NAD and elevated mitochondrial reactive oxygen species (mtROS) which leads to the development of spontaneous intestinal inflammation. These studies identify a previously unannotated nuclear-encoded lncRNA that localizes to the mitochondria and regulates metabolic activity to maintain intestinal homeostasis.

Provided herein are methods for diagnosing inflammatory bowel disease (IBD) in a subject. The methods comprise providing a sample from the subject; detecting a level of a HOXA11AS in the sample; and comparing the level of the HOXA11AS in the sample to a reference level; wherein presence of a level of HOXA11AS below the reference level indicates that the subject has IBD.

Also provided herein are methods comprising: providing a sample from the subject; and detecting a level of a HOXA11AS in the sample; and optionally comparing the level of the HOXA11AS in the sample to a reference level.

Further provided herein are methods of treating IBD in a subject. The methods comprise providing a sample from the subject; detecting a level of a HOXA11AS in the sample; comparing the level of the HOXA11AS in the sample to a reference level; wherein presence of a level of HOXA11AS below the reference level indicates that the subject has IBD; and administering a treatment for IBD to the subject who has a level of HOXA11AS below the reference level.

In some embodiments, the sample comprises nucleic acids or cells from the intestine, e.g., distal colon, of the subject. In some embodiments, the sample comprises an intestinal biopsy or cytology brushing.

In some embodiments, the HOXA11AS is at least 80%, 85%, 90%, 95%, or 99% identical to a HOXA11AS-1 or HOXA11AS-2 sequence shown in Table 2, e.g., SEQ ID NO:5 or 6.

In some embodiments, detecting a level of HOXA11AS comprises using RT-PCR.

Additionally, provided herein are methods for treating IBD in a subject. The methods comprise administering to the subject a HOXA11AS nucleic acid to cells in the intestine of the subject, e.g., to myeloid cells in the intestine of the subject. Also provided are HOXA11AS nucleic acids as described herein for use in treating IBD in a subject. In some embodiments, the IBD is ulcerative colitis (UC).

In some embodiments, the HOXA11AS is at least 80%, 85%, 90%, 95%, or 99% identical to a HOXA11AS-1 or HOXA11AS-2 sequence shown in Table 2, e.g., SEQ ID NO:5 or 6.

In some embodiments, the HOXA11AS nucleic acid is naked RNA or a viral vector comprising a sequence encoding HOXA11AS.

Additionally, provided herein are isolated nucleic acids encoding HOXA11AS, optionally wherein the HOXA11AS is at least 80%, 85%, 90%, 95%, or 99% identical to a HOXA11AS-1 or HOXA11AS-2 sequence shown in Table 2, e.g., SEQ ID NO:5 or 6.

In some embodiments, the isolated nucleic acid is linked to or comprised within a nanoparticle, e.g., a lipid nanoparticle, optionally wherein the isolated nucleic acid is a HOXA11AS RNA.

Also provided herein are expression vectors comprising a sequence encoding HOXA11AS operably linked to a promoter for expression of the HOXA11AS, optionally wherein the HOXA11AS is at least 80%, 85%, 90%, 95%, or 99% identical to a HOXA11AS-1 or HOXA11AS-2 sequence shown in Table 2, e.g., SEQ ID NO:5 or 6. In some embodiments, the expression vector is a viral vector, optionally an AAV, adenovirus, or lentivirus.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-H. HOXA11os/AS are highly abundant in the healthy colon and are downregulated in intestinal inflammation. Volcano plots representing differentially expressed lncRNAs in (A) resident macrophages and (B) infiltrated monocytes isolated from healthy and DSS treated mice. LncRNAs with p.value>0.05 are presented as grey dots (Data are represented as the mean of n=2-5 from 3 independent experiments). 24 (C) Combined HOXA11os reads from Oxford Nanopore sequencing (top), reference genome Gencode vM26 annotation (middle) and HOXA11os transcripts (bottom) in the colon (n=2). (D) RT-qPCR analysis of murine colonic HOXA11os transcripts in the indicated mice. Gene expression levels were normalized to TBP (***P<0.001, Data represent the mean±SEM of n=6-10). (E) HOXA11AS TPM expression levels from aligned RNA sequencing 26,27, n=62-228. HOXA11os/AS is predominantly expressed in the distal colon. (F) RT-qPCR analysis of HOXA11os mRNA in the indicated organs of WT mice (n=4). (G) RT-qPCR analysis of HOXA11os mRNA in the indicated organs of WT mice (n=4). (H) Representative images of single molecule RNAscope single-plex assay detecting endogenous HOXA11os in colon biopsies of WT mice.

FIGS. 2A-H. HOXA11os is localized to the mitochondria. RT-qPCR analysis of HOXA11os, GAPDH and 7SK expression levels in the cytoplasm and nucleus of (A) LP cells and (B) IECs isolated from the distal colon of WT mice presented as a % of total RNA (n=2). (C) Graphical summary of HOXA11os-binding proteins identified by ChIRP followed by mass spectrometry. (D) RT-qPCR analysis of HOXA11os, TBP and COXII expression levels in the cytoplasm and purified mitochondria. (Data are represented as the mean of n-3 from 2 independent experiments). Representative image of single molecule RNAscope multi-plex assay detecting endogenous (E) HOXA11os and mitochondria and (F) RNA polymerase II and Peptidylprolyl Isomerase B in colon biopsies of WT mice. (G) Percentage of HOXA11os positive mitochondria in the distal colon of WT mice. (***P<0.001, Data represent the mean±SEM of n=8-10 from 2-3 independent experiments). Arrows represent colocalization sites. (H) Representative images of single molecule RNAscope multi-plex assay detecting endogenous HOXA11os and mitochondria in the distal colon of HOXA11os-KO mice. Scale bar=10 μm.

FIGS. 3A-Q. HOXA11os-KO mice developed mild signs of spontaneous intestinal inflammation and are highly susceptible to colitis. Oxygen consumption rate of colonic cells isolated from HOXA11os-KO and WT littermate control mice at 4-months of age. (**P<0.01, Data represent the mean±SEM of n=2 from 5 independent experiments). RT-qPCR analysis of (B) IL-6, (C) IL-1ß and (D) RANTES expression levels in colons isolated from the indicated mice at 4-month of age. (**P<0.01, ***P<0.001, Data represent the mean±SEM of n=7-8). (E) Schematic illustration of acute model of DSS-inded colitis. (F,J) Colon length, (G,K) percentage weight change and (H,L) stool appearance of HOXA11os-KO and WT littermate control mice treated with DSS for 7 days. (***P<0.001, Data represent the mean±SEM of n=2-8 from 6 independent experiments). (I,M) Pathological score of H&E-stained colon sections from HOXA11os-KO and WT littermate control mice treated with DSS for 7 days. (***P<0.001, Data represent the mean±SEM of n=2-4 from 3 independent experiments). (N) Representative H&E-stained colon sections of the indicated mice. RT-qPCR analysis of (O) IL-6, (P) IL-1ß and (Q) CXCL-10 expression levels in colons isolated from the indicated mice treated with DSS for 7 days. (*P<0.05, **P<0.01, Data represent the mean±SEM of n=5-6 from 2 independent experiments).

FIGS. 4A-I. HOXA11os deficiency in the hematopoietic compartment promoted colitis. (A) Schematic illustration of bone marrow chimera experiments of the indicated irradiated (900R) mice reconstituted with 10⁷ bone marrow cells from donor mice. graphical summary of (B) weight loss, (C) colon length and (D) stool appearance of the indicated mice 4 days post DSS treatment. (*P<0.05, ***P<0.001. Data represent the mean±SEM of n=3-5 from 2 independent experiments). (F) Representative H&E-stained colon sections of the indicated mice. RT-qPCR analysis of (G) IL-6, (H) IL-1B and (I) CXCL-1 expression levels in colons isolated from the indicated mice 4 days post DSS treatment. (*P<0.05, **P<0.01. Data represent the mean±SEM of n=3-5 from 2 independent experiments).

FIGS. 5A-O. HOXA11os deficient immune cells displayed impaired complex I activity and dysfunctional mitochondria. (A) NAD+/NADH ratio, (B) ATP nM concentration and (C) Mitochondrial ROS concentrations in CD45⁺ LP immune cells isolated from the indicated mice at 4.5-months of age. (**P<0.01, ***P<0.001, Data represent the mean±SEM of n=3 from 2 independent experiments). (D) RT-qPCR analysis of mitochondrial DNA as mDNA/nDNA ratio of colonic LP CD45⁺ immune cells isolated from HOXA11os-KO and WT littermate control mice (*P<0.05, ***P<0.001, Date represents the mean±SEM of n=7). HOXA11os interacts with the complex I subunit, NDUFV1. (E) RT-qPCR analysis of HOXA11os and TBP expression levels in colonic HOXA11os-KO and WT LP cells of the indicated pulldowns. (***P<0.001, Data represent the mean±SEM of n=3 from 2 independent experiments). (F) Representative image of Western blotting of NDUFV1 and β-actin in colonic LP cells of HOXA11os-KO and WT littermate control mice with the indicated experimental groups. HOXA11os regulates complex I activity in LP myeloid cells. MitoTrackerGreen FMI analysis (G) and representative MFI images (H) of mitochondrial mass and Mito TrackerRed FMI analysis (I) and representative MFI images (J) of mitochondrial mass of colonic CD45⁺CD1b⁺ myeloid cells isolated from HOXA11os-KO and WT littermate control mice with the indicated treatment. (**P<0.01, ***P<0.001, Date represents the mean±SEM of n=2-4 from 3 independent experiments). RT-qPCR analysis of (K) IL-6, (L) IL-1B and (M) CXCL-10 expression levels in colonic myeloid cells isolated from the indicated mice (***P<0.001, Data represent the mean±SEM of n=4). (N) Representative Western blotting image of mitochondrial markers in colonic LP CD45⁺ cells isolated from HOXA11os-KO and WT littermate control mice. Equal loading was detected by measuring B-actin. n=5. (O) Representative images of colonic LP myeloid cells mitochondria obtained by transmission electron microscopy (TEM).

FIGS. 6A-I. Depletion of mtROS ameliorated colitis in HOXA11os-KO mice. (A) Schematic image illustration Rotenone experimental design. (B) Colon length, (C) percentage weight change, (D) stool appearance and (E) Pathological score of H&E-stained colon. (**P<0.01, ***P<0.001, Data represent the mean±SEM of n=3-6 from 2 independent experiments). (F) Representative H&E-stained colon sections of the indicated mice. RT-qPCR analysis of (G) IL-6, (H) IL-1ß and (I) CXCL-10 expression levels in colons isolated from the indicated mice (**P<0.01, ***P<0.001, Data represent the mean±SEM of n=3-5 from 2 independent experiments).

FIGS. 7A-H. HOXA11AS is highly abundant in the healthy colon and is downregulated in ulcerative colitis. Related to FIG. 1. (A) Combined HOXA11AS reads from Oxford Nanopore sequencing (top), reference. Gencode v39 annotation (middle) and HOXA11AS transcripts (bottom) in the colon. (B) RT-qPCR analysis of human colonic HOXA11AS transcripts in the indicated treatments. Gene expression levels were normalized to GAPDH (*P<0.05, Data represent the mean±SEM of n=5-6). (C) Schematic illustration of colonic HOXA11os and HOXA11AS transcripts and the percentage sequence conservation calculated by PFAAT. See also FIG. 12. (D) Representative image of Western blotting of Myc-tagged HOXA11os-potential ORFs and positive control Myc-tagged Caspase-1 in HEK293T cells, n=2. HOXA11os/AS is predominantly expressed in the distal colon. (E) HOXA11AS TPM expression level from aligned RNA sequencing 79,80 n=5-125. (F) RT-qPCR analysis of HOXA11os expression levels in CD45⁺ LP cells and CD45-IECs isolated from the distal colon. (n=8). (G) Representative image of single molecule RNAscope single-plex assay detecting endogenous HOXA11os (brown) in the indicated areas of colon biopsies of WT mice. (H) RT-qPCR analysis of HOXA11os expression levels in cells isolated from the distal colon of WT mice following the indicated treatments (Data are represented as mean±SEM of n=2-4 pulled mice from 2 independent experiments).

FIGS. 8A-F. HOXA11os-KO mice exhibit comparable expression levels of the protein coding gene HOXA11 as WT mice. Related to FIG. 1. (A) Schematic illustration of the CRISPR/Cas9 strategy to generate HOXA11os-KO mice (shown is SEQ ID NO:12). (B) RT-qPCR analysis of HOXA11os transcript 1 and HOXA11os transcript 2 expression levels in the distal colon of the indicated mice. (***P<0.001, Data represent the mean±SEM of n=4). (C) RTqPCR analysis of HOXA11os and HOXA11 protein expression levels in the distal colon of the indicated mice. (Data represent the mean±SEM of n=6). (D) Immunoblot analysis of HOXA11 protein and b-actin in lysates generated from colon tissue from the indicated mice. (E) RT-qPCR analysis of HOXA11os and HOXA11 protein expression levels in the distal colon of the indicated mice treated with 2% DSS for 7 days. (Data represent the mean±SEM of n=5). (F) Immunoblot analysis of HOXA11 protein and b-actin in lysates generated from colon tissue from the indicated mice treated with or without DSS.

FIGS. 9A-J. HOXA11os-KO mice demonstrated impaired recovery following DSS-induce colitis experiment. Related to FIG. 3. (A) Representative images of immunohistochemistry staining of Ki67 in distal colon biopsies collected from HOXA11os-KO and WT littermate control mice. (B) Quantification of Ki67 positive cells per field of view. (***P<0.001, Data represent the mean±SEM of n=2-17 from 2-3 independent experiments). Scale bar=100 μm. (C) Schematic image illustrating the recovery model of DSS-induced colitis. Graphical summary of (D) colon length, (E) stool appearance and (F) weight change and (G) pathological score of H&E-stained colon sections of HOXA11os-KO and WT littermate control mice 7 days post DSS treatment. (**P<0.05, ***P<0.001, Data represent the mean±SEM of n=2-8 from 3 independent experiments). (H) Representative H&E images of HOXA11os-KO and WT littermate control mice 7 days post DSS treatment. (I) pathological score of epithelial hyperplasia and pathological score of (J) epithelial erosion of of HOXA11os-KO and WT littermate control mice 7 days post DSS treatment. (*P<0.05, ***P<0.001, Data represent the mean±SEM of n=4-5 from 2 independent experiments).

FIGS. 10A-J S7. HOXA11os-KO mice are hyper susceptible to Salmonella Typhimurium-induced colitis. Related to FIG. 3. (A) Colon length, (B) percentage weight change, (C) stool appearance and (D) Pathological score of H&E-stained colon sections from pretreated streptomycin HOXA11os-KO and WT littermate control mice infected with Salmonella Typhimurium. (*P<0.05, ***P<0.001, Data represent the mean±SEM of n=5-7 from 3 independent experiments). (E) Representative H&E-stained colon sections of the indicated mice. RTqPCR analysis of (F) IL-6, (G) IL-1b and (H) CXCL-10 expression levels in colons isolated from the indicated mice (*P<0.05, ***P<0.001, Data represent the mean±SEM of n=5-7 from 3 independent experiments). HOXA11os function in the colon is independent on the microbiome. (I) RT-qPCR analysis of HOXA11os expression in the distal colon of WT mice treated for 4 weeks with a broad-spectrum antibiotic cocktail (ABX) or the indicated single antibiotic, ampicillin (1 gr/L), neomycin (1 gr/L), vancomycin (0.5 gr/L) and metronidazole (1 gr/L). (Data represent the mean±SEM of n=5 from 2 independent experiments). (J) Principal coordinates analysis (PCoA) plot of whole genome shotgun sequencing (WGS) community profiles representing diversity based on genotype using the weighted UniFrac distance metric, n=23-27.

FIGS. 11A-J. Rectal administration of complex I inhibitor to WT mice phenocopied colitis observed in HOXA11os-KO mice. Related to FIG. 6. (A) Schematic image illustration Rotenone experimental design. (B) Colon length, (C) percentage weight change, (D) stool appearance and (E) pathological score of H&E-stained colon. (**P<0.01, ***P<0.001, Data represent the mean±SEM of n=3-5 from 2 independent experiments). (F) Representative H&E-stained colon sections of the indicated mice. RT-qPCR analysis of (G) IL-6, (H) IL-1b and (I) CXCL-10 expression levels in colons isolated from the indicated mice (*P<0.05, **P<0.01, ***P<0.001, Data represent the mean±SEM of n=3-5 from 2 independent experiments). Depletion of mtROS ameliorated colitis in HOXA11os-KO mice. (J) Representative H&E-stained colon sections of the indicated mice.

FIG. 12. Sequence conservation. Alignment of mouse and human sequences. Shown are SEQ ID NO: 1, nucleotides 1-1523 of SEQ ID NO:2, SEQ ID NO:5, SEQ ID NO: 6, SEQ ID NO: 19, and SEQ ID NO:20, in order of appearance.

### DETAILED DESCRIPTION

New therapeutic targets are required to improve treatments and long-term outcomes for patients with Inflammatory Bowel Disease (IBD). While most studies have focused on delineating the functional roles of proteins in signaling pathways that initiate inflammation in the intestinal tract, a significant amount of genetic evidence suggests noncoding regions also plays important role in the regulation and progression of UC. Indeed, the majority of IBD-associated SNPs are located in noncoding genomic regions. LncRNAs account for the majority of RNA transcribed from noncoding genomic regions, accounting for a significant proportion of the human genome,¹⁻³ and have been implicated in the development of acute inflammation and chronic inflammatory diseases ⁴⁻¹². To date over 16,000 lncRNAs have been annotated in the human genome ¹³. Yet, the majority of these remain uncharacterized.

A small number of studies have revealed that lncRNA expression is associated with the pathogenesis of UC ^{14, 15}. LncRNAs have been implicated in UC through the regulation of miRNA expression ^{16, 17} or through modulation of intestinal epithelial barrier function ¹⁸. Given that these studies have primarily relied on cell-based in vitro assays and gene expression profiling ¹⁹⁻²³, physiological functions of lncRNAs in the pathogenesis of UC remain to be defined in detail, and thus their physiological role in UC is still unknown.

Described herein is a nuclear-encoded lncRNA, HOXA11os, that localizes to mitochondria to promote mitochondrial function and maintain intestinal homeostasis. HOXA11os is a tissue specific lncRNA that is highly conserved between mice and humans. Given the unique expression profile of HOXA11os in the distal colon, we performed all of our studies on physiologically relevant colon biopsies collected from mouse and human patients. LncRNAs are highly tissue specific ²⁹, and their annotation is poor and incomplete. Accurate annotation of lncRNAs is fundamental to define their biology. To overcome this limitation, we performed long read RNA sequencing on tissue biopsies which enabled the precise identification of the exact transcripts of HOXA11Aos expressed in the colon. This approach identified a previously unreported highly abundant transcript of HOXA11os.

Characterization of lncRNA subcellular localization and the proteins they interact with are central to understanding their function. Following identification of the specific HOXA11os transcripts expressed in the colon, we designed HOXA11os-specific probes to define the subcellular localization of HOXA11os. To date, lncRNAs have been shown to localize to the nucleus or cytoplasm. While studies have begun to interrogate the localization of lncRNAs to specific organelles, only a small portion of organelle-associated lncRNAs have been functionally characterized ³⁰.

Remarkably, HOXA11os was localized to mitochondria within cells of the distal colon and interacted with mitochondria residing proteins in shared metabolic pathways. To study the physiological function of HOXA11os in the colon, we generated HOXA11os deficient mice by introducing a minimal polyadenylation signal (pAS) downstream of the HOXA11os transcription start site. This approach efficiently terminated the transcription of HOXA11os in the distal colon while leaving the genomic locus intact. Interestingly, aged HOXA11os deficient mice demonstrated impaired mitochondrial OXPHOS in cells of the distal colon and impaired turnover of epithelial cells. HOXA11os deficient mice developed mild spontaneous intestinal inflammation and were hyper susceptible to experimental models of colitis. These data suggest that HOXA11os functions to restrict age associated susceptibility to intestinal inflammation by maintaining optimal functional mitochondria in cells of the distal colon.

Mitochondrial dysfunction has long been associated with intestinal inflammation. Indeed, mice carrying mitochondrial polymorphisms associated with higher OXPHOS activity and ATP production are protected from colitis ⁵⁵. Mitochondrial dysfunction has also been associated with human UC. A decrease in OXPHOS gene expression ^{39-41,56}, deficiencies in electron transport chain activity and elevated mtROS have all been reported in patients with UC ⁵⁷⁻⁵⁹. These data suggest that mitochondrial dysfunction is a key pathogenic feature of UC. mtROS plays an important role in host defense. Yet, under pathological conditions, high mtROS levels can lead to oxidative damage and inflammation ^{60, 61}. Furthermore, excessive mtROS levels can triggers colitis, whereas mtROS inhibitors alleviate colitis in mice ⁶².

HOXA11os was found at high levels in both the hematopoietic and non-hematopoietic cells of the distal colon and deficiency of HOXA11os in both cellular compartments resulted in impaired complex I activity, characterized by decreased NAD production and elevated mtROS. Yet, bone marrow chimera experiments revealed that complex I deficiency in the hematopoietic compartment but not the non-hematopoietic compartment was the cause of colitis susceptibility. This suggest that immune cells in the colon may be more susceptible to dysfunctional complex I activity when compared to IECs due to slower cellular turnover or due to different metabolic requirements that are unique to myeloid cells. HOXA11os-KO immune cells displayed dysregulation of the complex I subunit, NDUFV1 and a decrease in the protein levels of markers of mitochondrial mass and biogenesis. HOXA11os-KO immune cells also displayed elevated levels of the mitophagy regulator PARKIN. Rotenone treated colonic cells from WT mice mirrored the effects of HOXA11os deficiency on these dynamic markers. Immune cells isolated from HOXA11os-KO mice demonstrated comparable protein levels to cells isolated from HOXA11os-KO mice treated with rotenone. These data strongly support our conclusions that HOXA11os exhibits its function via the regulation of complex I in colonic immune cells.

Through the use of bone marrow chimeras, we defined immune cells as key drivers of colitis susceptibility in HOXA11os-KO mice. HOXA11os-KO myeloid cells displayed decreased ΔΨₘ and a disrupted mitochondrial ultrastructure. Comparable levels of ΔΨₘ were observed in HOXA11os-KO myeloid cells treated with or without rotenone, further supporting the role of complex I impairment as the key mediator of colitis in HOXA11os deficient mice. Rotenone treatment of WT mice phenocopied the colitis phenotype observed in HOXA11os-KO mice. Additionally, inhibition of mtROS ameliorated colitis in HOXA11os-KO mice. Collectively these findings underscore the importance of HOXA11os as a regulator of complex I activity in colonic myeloid cells. HOXA11os likely regulates complex 1 activity by regulating protein-protein interactions, enhancing substrate binding or through regulation of catalytic activity.

In summary, we have identified a previously unknown mechanism by which the lncRNA HOXA11os regulates cell metabolism to maintain intestinal homeostasis. The unique regulatory function of HOXA11os in the colon emphasizes the importance of noncoding genomic regions in the regulation of UC. This study provides the first characterization of how a lncRNA regulates cell metabolism at a tissue level and how its dysregulation can impact chronic diseases, such as UC. HOXA11os is abundant in the healthy colon and significantly reduced in colitis.

Thus, provided herein are gene replacement approaches to restore HOXA11AS (the human homolog of HOXA11os) in the colon for patients with IBD, e.g., with UC. Further, since HOXA11os levels inversely correlate with IBD/UC disease severity this lncRNA can also be used as a sensitive colon specific disease relevant biomarker.

### Methods of Diagnosis

Included herein are methods for diagnosing an inflammatory bowel disease in a subject. The methods rely on detection of levels of HOXA11AS. The methods include obtaining a sample from a subject, and evaluating the presence and/or level of HOXA11AS in the sample, and comparing the presence and/or level with one or more references, e.g., a control reference that represents a normal level of HOXA11AS, e.g., a level in an unaffected subject, and/or a disease reference that represents a level of the proteins associated with IBD, e.g., a level in a subject having IBD, e.g., CD or UC.

As used herein the term “sample”, when referring to the material to be tested for the presence of a biological marker using the method of the invention, includes inter alia tissue (e.g., from a biopsy), intestinal brushings, and stool samples. Various methods are well known within the art for the identification and/or isolation and/or purification of a biological marker from a sample. An “isolated” or “purified” biological marker is substantially free of cellular material or other contaminants from the cell or tissue source from which the biological marker is derived, i.e., partially or completely altered or removed from the natural state through human intervention. For example, nucleic acids contained in the sample can be isolated according to standard methods, for example using lytic enzymes, chemical solutions, or isolated by nucleic acid-binding resins following the manufacturer's instructions.

The presence and/or level of a HOXA11AS nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of HOXA11AS. Measurement of the level of a biomarker can be direct or indirect. For example, the abundance levels of HOXA11AS can be directly quantitated.

RT-PCR can be used to determine levels of HOXA11AS. The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes are most commonly used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, the presence and/or level of HOXA11AS is comparable to the presence and/or level of the protein(s) in the disease reference, and the subject has one or more symptoms associated with IBD then the subject has IBD. In some embodiments, the subject has no overt signs or symptoms of IBD, but the presence and/or level of one or more of the proteins evaluated is comparable to the presence and/or level of the protein(s) in the disease reference, then the subject has an increased risk of developing IBD. In some embodiments, once it has been determined that a person has IBD, or has an increased risk of developing IBD, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of HOXA11AS, e.g., a control reference level that represents a normal level of HOXA11AS, e.g., a level in an unaffected subject or a subject who is not at risk of developing a disease described herein, and/or a disease reference that represents a level of the proteins associated with conditions associated with IBD, e.g., a level in a subject having IBD (e.g., CD or UC).

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g., IBD).

A disease reference subject is one who has (or has an increased risk of developing) IBD. An increased risk is defined as a risk above the risk of subjects in the general population.

Thus, in some cases the level of HOXA11AS in a subject being less than or equal to a reference level of HOXA11AS is indicative of a clinical status (e.g., indicative of a disorder as described herein, e.g., IBD. In other cases the level of HOXA11AS in a subject being greater than or equal to the reference level of HOXA11AS is indicative of the absence of disease or normal risk of the disease. In some embodiments, the amount by which the level in the subject is the less than the reference level is sufficient to distinguish a subject from a control subject, and optionally is a statistically significantly less than the level in a control subject. In cases where the level of HOXA11AS in a subject being equal to the reference level of HOXA11AS, the “being equal” refers to being approximately equal (e.g., not statistically different). In some embodiments, the level of HOXA11AS is compared to a range of reference levels that represent varying severities of IBD, wherein lower levels of HOXA11AS correlate with more severe forms of IBD.

The predetermined value can depend upon the particular population of subjects (e.g., human subjects) selected. For example, an apparently healthy population will have a different ‘normal’ range of levels of HOXA11AS than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

### Methods of Treatment

The methods described herein include methods for the treatment of disorders associated with inflammation in the colon. In some embodiments, the disorder is inflammatory bowel disease (IBD). IBD is a collective term that describes conditions associated with chronic or recurring immune response and inflammation of the gastrointestinal (GI) tract. The two most common inflammatory bowel diseases are ulcerative colitis (UC) and Crohn's disease (CD). Both are marked symptoms of IBD, for example, severe diarrhea, abdominal pain, fatigue, and weight loss. IBD and other gastrointestinal inflammatory diseases can be debilitating and sometimes lead to life-threatening complications.

As used in this context, to “treat” means to ameliorate at least one symptom of the disorder associated with inflammation in the colon. Often, these disorders result in diarrhea, abdominal pain, fatigue, and weight loss; thus, administration of a therapeutically effective amount of a treatment can result in one or more of a reduction in diarrhea, abdominal pain, fatigue, and weight loss and a return or approach to normal stools, cessation of pain, lessening of fatigue, and weight gain or stabilization.

In some embodiments, the methods include administering a therapeutically effective amount of a nucleic acid encoding HOXA11AS as described herein, to a subject who is in need of, or who has been determined to be in need of, such treatment.

In some embodiments, the methods include administering a treatment for IBD as known in the art to a subject identified as being in need thereof using a method described herein. Such treatments can include administration of aminosalicylates (e.g., sulfasalazine (SASP) and other types of 5-aminosalicylic acid (5-ASA) drugs), corticosteroids (CSs, e.g., budesonide, beclomethasone dipropionate), immunomodulators (e.g., thiopurines (TPs), methotrexate (MTX), calcineurin inhibitors, and Janus Kinase (JAK) inhibitors), biologics (e.g., inhibitors of pro-inflammatory cytokines TNF-α (e.g., infliximab (IFX), adalimumab (ADA), golimumab) and IL-12/IL-23 (e.g., ustekinumab, mirikizumab, risankizumab, and integrin antagonists (e.g., vedolizumab, etrolizumab, carotegrast Methyl (AJM300), and PF-00547659), and oral small molecules (e.g., JAK inhibitors such as tofacitinib, filgotinib, upadacitinib, deucravacitinib; sphingosine-1-phosphate receptor modulators and agonists, e.g., ozanimod or etrasimod). In some embodiments, the treatment includes resection, e.g., laparoscopic resection; apheresis therapy (e.g., Adsorptive granulocyte/monocyte apheresis (GMA)); antibiotics, probiotics, prebiotics, postbiotics, synbiotics, and fecal microbiota transplantation (FMT); and stem cell therapy comprising administration of haematopoietic stem cells (HSCs), mesenchymal stem cells (MSCs), and intestinal stem cells (ISCs). See, e.g., Cai et al., Front Med (Lausanne). 2021; 8:765474.

### HOXA11AS Nucleic Acids

The HOXA11AS nucleic acids described herein, e.g., nucleic acids encoding HOXA11AS, can be incorporated into a gene construct to be used as a part of a gene therapy protocol. The invention includes targeted expression vectors for in vivo transfection and expression of HOXA11AS in particular cell types, especially myeloid cells of the gut. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vitro or in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques.

Protocols for producing recombinant viruses and for infecting cells in vitro or in vivo with such viruses are known in the art; examples can be found in Ausubel, et al., eds., Gene Therapy Protocols Volume 1: Production and In Vivo Applications of Gene Transfer Vectors, Humana Press, (2008), pp. 1-32; Ghosh et al., Viral Vector Systems for Gene Therapy: A Comprehensive Literature Review of Progress and Biosafety Challenges, Applied Biosafety. March 2020. 7-18; and other laboratory manuals.

Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986). Adenoviral delivery of nucleic acids to the intestine has been reported; see, e.g., Cheng et al. Hum Gene Ther. 1997; 8:755-64; Foreman et al. Hum Gene Ther. 1998; 9:1313-21; Hogaboam et al., J Clin Invest. 1997 Dec. 1; 100(11):2766-76.

A viral vector system particularly useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and Mclaughlin et al., J. Virol. 62:1963-1973 (1989)). AAV vectors, such as AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression.

Thus, in some embodiments, the HOXA11AS nucleic acid is present in a vector for gene therapy, such as an AAV vector. In some instances, the AAV vector is selected from the group consisting of AAV1, AAV2, AAV3, AAV4, AAV5, AAV6, AAV7, AAV8, AAV9, AAVrh10, AAV11, and AAV12. The use of AAV vectors to deliver constructs for expression in the intestinal tract has been described; see, e.g., Shao et al., J Pediatr Gastroenterol Nutr 43:168-179 (oral administration of AAV2); Polyak et al., Dig Dis Sci. 2008 May; 53(5): 1261-1270 (Gene delivery to intestinal epithelial cells in vitro and in vivo with recombinant AAV11, 2 and 5/AAV pseudotypes 2/1, 2/2, and 2/5); During et al., Nature Medicine 4:1131-1135 (1998) (orally administered AAV for gene therapy of lactose intolerance); Buckinx and Timmermans, Histochemistry and Cell Biology volume 146, pages 709-720 (2016); and Polyak et al., Am J Physiol Gastrointest Liver Physiol. 2012 Feb. 1; 302(3):G296-308 (AAVrh10/AAV pseudotypes 4, 7, 8, 9, and 10).

An AAV vector as described herein can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more neurons or groups of cells. In addition, the capsid can be altered to include one or more peptides that increase expression in the CNS, see, e.g., Yao et al., Nat Biomed Eng. 2022 Oct. 10. doi: 10.1038/s41551-022-00938-7; Chatterjee et al., Gene Ther. 2022 June; 29(6):390-397; Meng et al., Mol Ther Methods Clin Dev. 2021 Feb. 27; 21:28-41; Zhang et al., Biomaterials. 2022 February; 281:121340; Gray, Cell Gene Ther. Insights 5, 1361-1368 (2019); Nonnenmacher et al., Mol. Ther. Methods Clin. Dev. 20, 366-378 (2021).

Without limitation, illustrative examples of pseudotyped AAV vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. In particular instances, the present disclosures can include a pseudotyped AAV9 or AAVrh10 viral vector including a nucleic acid as disclosed herein. See Viral Vectors for Gene Therapy: Methods and Protocols, ed. Machida, Humana Press, 2003.

In some instances, a particular AAV serotype vector may be selected based upon the intended use, e.g., based upon the intended route of administration.

AAV vectors preferably include inverted terminal repeats (ITRs); promoters, enhancers (e.g., CMV enhancer), other cis-regulatory elements, and/or capsid serotype variants that control and drive expression of HOXA11AS. With regard to promoters, vectors can include promoters that drive expression in many cell types (e.g., human β-actin, human elongation factor-la, chicken β-actin combined with cytomegalovirus early enhancer, cytomegalovirus (CMV), simian virus 40 (SC40), herpes simplex virus thymidine kinase (HSVTK), PGK, CAG, sCAG, or CASI promoters) or specifically in myeloid cells, e.g., mfap4 promoter, CSF1R macrophage promoter CD68 promoter, or a synthetic promoter derived from elements of transcription factor genes (Levin et al., Gene Therapy 19:1041-1047 (2012)); or in epithelial cells of the intestinal tract, e.g., T3(b) promoter (Aihara et al., FEBS Lett. 1999 Dec. 10; 463(1-2):185-8); human intestinal fatty acid binding protein promoter (HIFABP), rat intestinal fatty acid binding protein promoter (RIFABP), human mucin-2 promoter (HMUC2), human lysozyme promoter (HLY), human sucrase-isomaltase enhancer (HSI); and rat intestinal trefoil factor (RITF)(Ya-Feng et al., J Anim Sci Biotechnol. 2012; 3(1): 32). Other cis-regulatory elements can include posttranscriptional regulatory elements; 2A enhancers; polyadenylation sequences; and/or an intron. Posttranscriptional regulatory elements can include HBV Posttranscriptional Regulatory Element (HPRE), woodchuck hepatitis virus post-transcriptional regulatory element (WPRE) or variants thereof (e.g., WPRE2, WPRE3 see, e.g., Kalev-Zylinska M L, During M J J Neurosci. 2007 Sep. 26; 27(39):10456-67; Zanta-Boussif et al., Gene Therapy (16): 605-619 (2009); Choi et al., Mol Brain. 7:17 (2014); U.S. Pat. No. 6,136,597).

Various methods for application of AAV vector constructs in gene therapy are known in the art, including methods of modification, purification, and preparation for administration to human subjects (see, e.g., Viral Vectors for Gene Therapy: Methods and Protocols, ed. Machida, Humana Press, 2003). In addition, AAV based gene therapy targeted to cells of the CNS has been described (see, e.g., U.S. Pat. Nos. 6,180,613 and 6,503,888). High titer AAV preparations can be produced using techniques known in the art, e.g., as described in U.S. Pat. No. 5,658,776

A vector construct refers to a polynucleotide molecule including all or a portion of a viral genome and a transgene. In some instances, gene transfer can be mediated by a DNA viral vector, such as an adenovirus (Ad) or adeno-associated virus (AAV). Other vectors useful in methods of gene therapy are known in the art. For example, a construct as disclosed herein can include an alphavirus, herpesvirus, retrovirus, lentivirus, or vaccinia virus.

Also provided herein are methods of making the AAV, as well as host cells comprising the AAV. Methods for obtaining recombinant AAVs having a desired capsid protein are well known in the art. (See, e.g., US 2003/0138772 and WO 2019/200286, the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins.

In addition to viral transfer methods, such as those illustrated above, non-viral methods can also be employed to cause expression of a HOXA11AS nucleic acid in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

For example, nucleic acids comprising DNA or mRNA encoding HOXA11AS, or HOXA11AS RNA, can be delivered to cells directly, e.g., encapsulated in a delivery vehicle, e.g., lipids and lipid nanoparticles (LNP); polymers and polymer-based nanoparticles (see Paunovska et al., Nature Reviews Genetics volume 23, pages 265-280 (2022)). Delivery vehicles such as liposomes, nanocapsules, microparticles, microspheres, lipid particles, vesicles, and the like, may be used for the introduction of the compositions of the present disclosure into suitable host cells. In particular, the AAV or HOXA11AS nucleic acids can be formulated for delivery encapsulated in a lipid particle, a liposome, a vesicle, a nanosphere, or a nanoparticle (e.g., conjugated to a nanoparticle) or the like. See, e.g., Tanaka et al., Heliyon. 2018 December; 4(12): e00959.

In some embodiments, a HOXA11AS nucleic acid is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the HOXA11AS gene delivery systems can be introduced into a subject by any of a number of methods, each of which is familiar in the art. For instance, a pharmaceutical preparation of the gene delivery system can be introduced systemically, e.g., by intravenous injection, oral administration, or and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced orally, luminally, by catheter (see, e.g., Prieto et al., Gut. 2003 May; 52(Suppl 2): ii49-ii54).

In some embodiments, the pharmaceutical preparation of the gene therapy construct can consist essentially of the gene delivery system in an acceptable diluent or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

Also provided herein are compositions and formulations comprising the HOXA11AS nucleic acids and/or HOXA11AS encoding vectors, e.g., in a sterile carrier. Formulation of pharmaceutically-acceptable excipients and carrier solutions is well-known to those of skill in the art. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

### Exemplary sequences

In some embodiments, the sequence of a HOXA11AS nucleic acid used in a composition or method described herein is at least 80%, 85%, 90%, 95%, 97%, 98%, or 99% identical to a sequence set forth herein, e.g., a sequence shown in Table 2, e.g., SEQ ID NO:5 or 6. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

### EXAMPLES

The invention is further described in the following examples, which do not limit the scope of the invention described in the claims.

### Methods and Materials

The following materials and methods were used in the Examples set forth below.

### Experimental Model and Study Participant Details

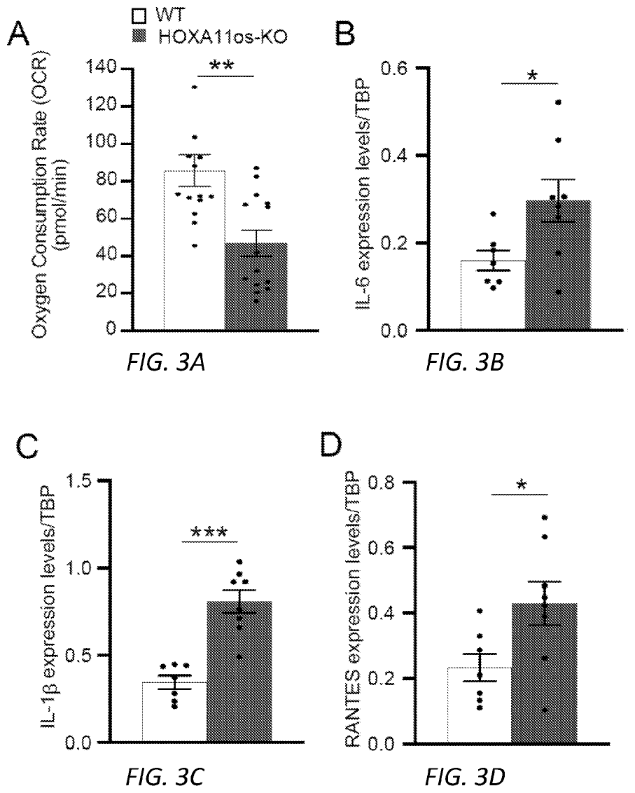

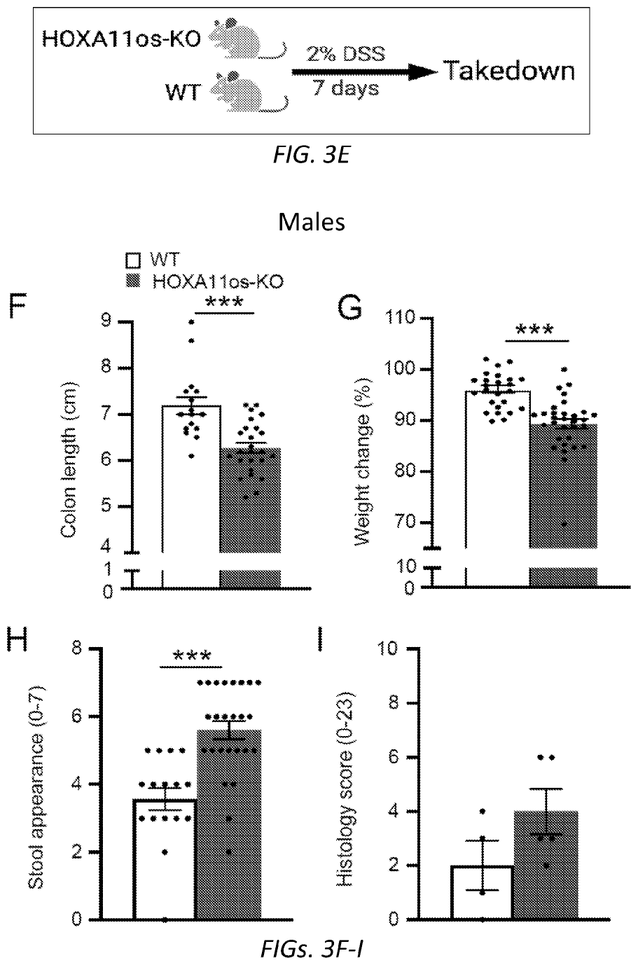

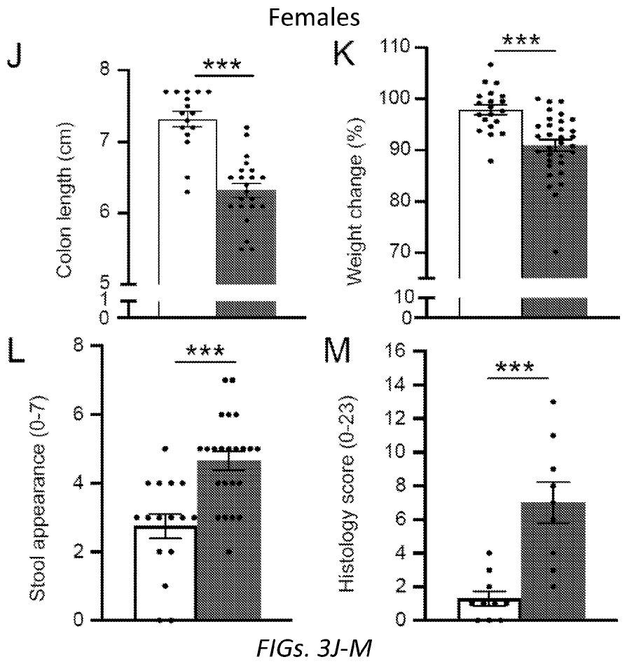

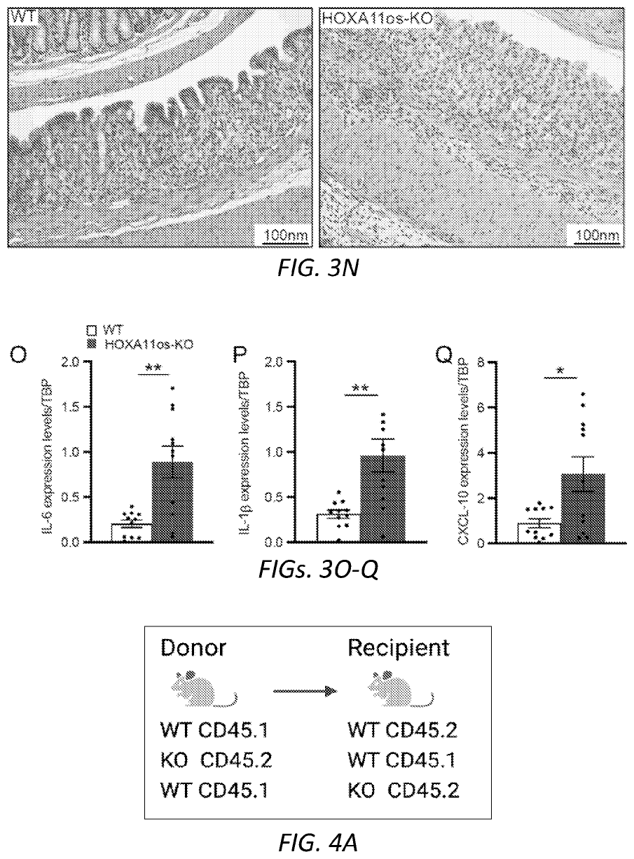

Animal studies. All animal experiments were approved by the Institutional Animal Care Use Committees at the UMass Chan Medical School. Animal were kept in specific pathogen free (SPF) environment. HOXA11os mice were generated on the C57BL/6 background by the insertion of synthetic poly adenylated, poly(A), site 33 to the first exon using CRISPR-Cas9 technology (Extended Data FIG. 3A). The design and synthesis of gRNAs were kindly provided by Prof. Jorge Henao-Mejia from the University of Pennsylvania.

Genotyping of HOXA11os-KO mice was performed in Transnetyx using TaqMan™ qPCR assay.

For the identification of HOXA11os-KO in mice, the following primers were used.

HOXA11os-KO Forward:
(SEQ ID NO: 13)
GCTCAGCTACATGGTCCTGGTC
HOXA11os-KO Reverse:
(SEQ ID NO: 14)
TCCGGGACCTGAAGCTATACA
HOXA11os-KO Reporter:
(SEQ ID NO: 15)
CATTAGATCTGTGTGTTGGTT

For the identification of HOXA11os-WT in mice, the following primers were used.

HOXA11os-WT Forward
(SEQ ID NO: 16)
GCAGCTCTCCTTTGCTCAGCTACA
HOXA11os-WT Reverse
(SEQ ID NO: 17)
CTCTCTTGCCGCCCTCA
HOXA11os-WT Reporter
(SEQ ID NO: 18)
CACTCGTGGACCAGG

Mice were generated by crossing heterozygous male mice with heterozygous littermate control female mice. Co-housed WT littermate control mice were used throughout the study. No gender bias was observed for intestinal inflammation and both male and female mice were used. Group sizes are specified in the figure legends. Animals were used solely for experiments described in this study and were not subjected to any treatment unless mentioned otherwise. Mice were co-housed with no more than 5 mice per cage.

Human subjects. Intestine biopsies collected from de-identified patients with ulcerative colitis and healthy individuals were used in this study. Biopsies samples were collected and stored in −80° C. until used.

DSS-induced acute colitis. For acute model of colitis, HOXA11os-KO and WT littermate control mice were co-house and treated with 2% DSS in the drinking water for 7 days. For recovery model of colitis, HOXA11os-KO and WT littermate control mice were treated with 1.5% DSS in the drinking water for 5 days followed by regular water for 7 days.

Salmonella-induced colitis. HOXA11os-KO and WT littermate control mice were pretreated with 20 mg streptomycin for 1 day following by administration of S. typhimurium (SL1344) by gavage (1.8*10\hat{ }8 CFU). Colon samples were extracted 4 days post infection.

MitoQ in vivo model. HOXA11os-KO and WT littermate controls were co-house and treated with 2% DSS in the drinking water for 7 days. An amount of 5 mg/kg MitoQ was administrated by intraperitoneal every other day during the duration of DSS treatment.

Rotenone in vivo model. HOXA11os-KO and WT littermate control were co-house and treated with 2% DSS in the drinking water for 7 days. An amount of 1 mg/kg of rotenone was rectally every other day during the duration of DSS treatment.

Immunoblotting. For immunoblot analysis cells were lysed in NP-40 lysis buffer (50 mM Tris-HCl, pH 7.4, containing 150 mM NaCl, 0.5% (w/v) IgePal, 50 mM NaF, 1 mM Na3VO4, 1 mM dithiothreitol) containing 1 mM phenylmethylsulfonyl fluoride and protease inhibitor cocktail (ThermoFisher). For analysis of whole colon, tissue was first sonicated in lysis buffer and protein concentration determined by DC protein assay kit (Bio Rad). For analysis of LP and IECs, cells were sonicated in RIPA buffer (150 mM NaCl, 50 mM Tris HCl pH 7.5, 0.1% SDS, 1% Triton, 0.5% deoxyxholate) containing protease inhibitor cocktail (ThermoFisher). Samples were resolved by using SDS-PAGE and transferred to nitrocellulose membranes and analyzed by immunoblot with the indicated antibodies. Immunoreactivity was visualized by the Odyssey Imaging System (LICOR Biosciences).

Lamina propria and Intestinal epithelial cells extraction. For extraction of IECs, colon samples were added to Hanks' Balanced Salt Solution (HBSS) containing 2.5 mM EDTA, 1 mM DTT and 5% of fetal calf serum and incubated at 37° C. with 300 RPM for 30 minutes. Remaining colon pieces were then washed with PBS and further digested for LP cell extraction in HBSS containing 10% fecal calf serum, 0.5 mg/ml collagenase I (Gibco), 0.5 mg/ml collagenase IV (Millipore Sigma) and 0.1 mg/ml DNase I (Roche) at 37° C. with 300 RPM for 30 minutes. Samples were then passed through 100 μm mesh and cells were collected by centrifugation at 900(×g) for 5 minutes. Cells were then subjected to MACS bead column-based separation. Cells were subjected to CD45 MACS MicroBeads (Mihtenyi Biotec) for CD45 positive LP cells and CD45 negative IECs.

cDNA synthesis and real time quantitative PCR. Total RNA was extracted from whole colon and lug of RNA was reverse transcribed using the iScript cDNA synthesis kit (Bio Rad). 5 ng of cDNA was then subjected to qPCR analysis using iQ SYBR Green super-mix reagent (Bio Rad). Murine and human gene expression levels were normalized to TATA-binding protein (TBP) and GAPDH, respectively. Relative mRNA expression was calculated by a change in cycling threshold method as 2-ddC (t) Specificity of RT-qPCR amplification was assessed by melting curve analysis.

Colitis score. Colitis scoring was carried out blindly on freshly collected fecal samples using the following criteria: stool consistency: 0-well-formed pellets, 1-changed formed pellets, 2-loose stool, 3-diarrhea, occult: 0-no blood, 1-traces of blood, 2-moderate, 3-severe, 4-bleeding from anus.

Histology. Colon tissues were fixed in 10% neutral buffered formalin for 24-48 hours before being processed and embedded in paraffin. Five micrometer thin sections were stained by H&E in an automated stainer (Leica Autostainer XL). Histomorphology of each H&E slide was evaluated by Applied Pathology Systems LLC at low and high-power field on an Olympus BX40 microscope, and the images were captured with Olympus cellSens Entry software at ×4 magnifications. Grading of histology scores was performed blindly by Applied Pathology Systems LLC under the following scoring criteria. Inflammatory cell infiltrate was evaluated by the number of leukocyte foci: 0—no significant change, 1—mild, infiltrated leukocytes in focal or occasional, 2—moderate, infiltrated leukocytes with more than one focus and 3—severe, infiltrated leukocytes diffuse or continuous. Epithelial changes in goblet cell loss were evaluated by the reduction of goblet cell numbers relative to baseline goblet cell numbers per crypt: 0—no significant change, 1—goblet cell loss of 10% of normal amount, 2—goblet cell loss of 10-50% and 3—goblet cell loss higher than 50%. Crypt abscesses were evaluated by the neutrophils in crypt lumen: 0—no crypt abscess, 1—rare crypt abscesses, 2—multiple crypt abscesses and 3—continuous crypt abscesses. Erosion was evaluated by the loss of surface epithelium: 0—no erosion, 1—one focus, 2—multiple foci and 3—continuous surface loss. Hyperplasia was evaluated by the increase in crypt number or length relative to baseline, visible as crypt elongation or thickened crypt layers: 0—no hyperplasia, 1—mild, increase 50%, 2—moderate, increase of 50-100% and 3—marked, increase 100%. Ulceration was evaluated by crypt loss reaching beyond muscularis mucosae: 0—no ulceration, 1—one focus, 2—multiple foci and 3—continuous. Lymphoplasmacytic aggregates were evaluated by a score based on the location of lymphoplasmacytic aggregates reach: 0—no lymphoplasmacytic aggregates, 1—lymphoplasmacytic aggregates reach mucosa, 2—lymphoplasmacytic aggregates reach submucosa and 3—lymphoplasmacytic aggregates reach muscularis propria or subserosa.

Ki67 staining. Immunohistochemistry stain was performed by Applied Pathology Systems using detection kit (Vector Laboratories, MP-7601) on Dako autostainer. Paraffin tissues sectioned at 5-micron thickness were dewaxed, rehydrated, and subjected to the antigen retrieval in citrate-based buffer pH 6.0 in a pressure cook. Slides were blocked with BloxAll blocking buffer and 2.5% Horse Serum respectively prior to 1 hour incubation with anti-Ki67 antibody at 1:250 dilution (abcam, ab16667). Subsequently, the sections were incubated with anti-rabbit Amplifier antibody and ImmPress Excel polymer reagent sequentially before applying with DAB chromogen. The slides were then counterstained with hematoxylin followed by dehydration and coverslipping.

Flow cytometry. LP and IE cells were stained with anti-CD45.2 BV650, anti-CD11b BV510, anti-Ly6C APC, anti-CD11c eF450, anti-CD64 BV711, anti-Ly6G PE-cy7, anti-Ly6G FITC, anti-CD3 PerCP-ef710, anti-B220 PE-Cy5, anti-CD326 APC. For mitochondrial mass and membrane potential, cells were stained with 120 nM MitoTracker™ Green and MitoTracker™ Red (ThermoFisher), respectively. Cells were acquired on a Cytek™ Aurora technology. Flow cytometry analysis was done with the FlowJo software. For sorting of colonic cells, cells were ectracted as described under sterile conditions and sorted directly into complemented growing media. Sorting was performed on FACSAria III or FACSAria Fusion.

Antibiotic-induced microbiota depletion. 2-month-old WT mice were treated with autoclaved drinking water or autoclaved drinking water supplemented with 1 gr/L neomycin, 1 gr/L metronidazole, 0.5 gr/L vancomycin and 1 gr/L ampicillin for 1 month. Antibiotics were given individually or as a cocktail containing all 4 antibiotics. Fecal pellets were collected, and total DNA was extracted using DNeasy PowerSoil Kit (Qiagen) and subjected to qPCR using universal primers referred to as the 319F and 806R that hybridize to the V3-V4 region of the rRNA gene.

Microbiome sequencing. Whole-genome shotgun (WGS) DNA sequencing libraries were constructed using Nextera XT DNA Library Prep Kits (Illumina Inc., San Diego, CA) and sequenced on a NextSeq500 Sequencing System as 150-base paired-end reads. Reads were trimmed reads and removed host decontamination using Trimmomatic and Bowtie2^{63, 64} using Mus musculus genome assembly mm10 as the host reference. Reads were profiled for bacterial spices abundances using MetaPhlAn version 3.0.1 and database mpa_v292_CHOCOPhlAn_20190165.

Oxford Nanopore sequencing. Libraries for long-read Nanopore sequencing were generated with RNA from healthy distal colon. For mouse samples, RNA was enriched for poly (A) RNA using NEBNext Poly (A) mRNA Magnetic Isolation Module (NEB) and libraries were prepared using direct cDNA sequencing (SQK-DCS109) kits according to the manufacturer's instructions. Due to limitation in RNA, libraries from human samples were prepared using cDNA-PCR sequencing (SOK-PCS109) kit. Libraries were sequenced with a MinION 1B device on an R9.4.1 flow cells (all Oxford Nanopore Technologies). Base calling was performed using guppy v4.2.2 (high accuracy) on Tesla V100 gpu (Nvidia). Full length reads were identified using pychopper v2.5.0 and filtered reads were aligned to the murine genome (assembly GRCm39/mm39) or the human genome (assembly GRCh38/hg38) using minimap2 v2.17 with the parameters ‘-ax splice-uf-k14’. Annotations were generated using stringtie v2.1.5 with the parameters ‘-g 200-L--conservative’ based on the aligned Nanopore reads and supported by Gencode annotation vM26 (mouse) or v38 (human). Aligned reads and annotations were visualized using IGV v2.9.4.

RNAseq analysis. Murine RNA sequencing dataset reported in this article can be found at the gene expression omnibus (ncbi.nlm.nih.gov/geo/query/acc.cgi?acc=GSE140788)²⁴. RNA sequencing data was quantified with Salmon⁶⁶ (v1.9.0) using default parameters with respect to the concatenated GRCm39 cDNA and ncRNA transcriptome downloaded from ensemble⁶⁷ (release 109) without decoy sequences. Differential expression was computed with DESeq2⁶⁸ (v3.16) using the design formula (~batch+condition) in order to control for batch effects and genes with very low expression (rowCounts<10) were pre-filtered. Volcano plots were plotted using R (EnhancedVolcano package, https://github.com/kevinblighe/EnhancedVolcano.) with lfcShrinkage using apeglm⁶⁹, based on an adjusted p-value alpha of 0.05 and log 2 fold-change threshold of 1. Heatmaps and clusters were generated using R (pheatmap package) with hierarchal clustering on row-normalized and DESeq2 r log-normalized genes counts based on Euclidean distance (complete), after selecting for genes with an adjusted p-value<0.01 and |log 2 (FC)|≥2.

Human IBD cohort dataset used in this study was the Mount Sinai Crohn's and Colitis Registry (MSCCR) of biopsy whole transcriptome sequencing data of ~1200 patients enrolled from December 2013-September 2016 and approved by the Icahn School of Medicine at Mount Sinai Institutional Review Board^{26, 27}. Fastq files were trimmed using cutadapt using the options “-a AGATCGGAAGAGCACACGTCTGAACTCCAGTCA—minimum-length 1-j 15”⁷⁰ (SEQ ID NO:18). The trimmed fastq files were pseudoaligned and quantified using Salmon version 1.5.2⁶⁶. Salmon results files were imported into R using the package tximeta Bioconductor release 3.14⁷¹. Gene level counts were generated using the function summarizeToGene followed by TPM-normalization. The epithelial cell Seurat data object of single cell RNA-seq was retrieved from doi.org/10.1016% 2Fj.cell.2019.06.029 and subset to control and uninflamed samples. The percentage of epithelial cells with HOXA11AS expression greater than or equal to 1 TPM was compared to the total number of cells captured for each cell-type and the pre-existing epithelial cell identities were used.

In vitro translation assay. PcDNA3.1 (+)-C-Myc vectors coding for HOXA11os potential ORFs were purchased from GenScript, USA. pCI-Caspasel vector was used as positive control⁷². For transient expression, 4×10⁵ HEK293T cells were transfected with 0.5 μg of the indicated vectors using Lipofectamine™ 2000 (Invitrogen) as per manufacturer's instructions. Cells were lysate in RIPA buffer supplemented with protease inhibitor cocktail 24 hours post infection. Samples were resolved on 12% SDS-PAGE and transferred to nitrocellulose membranes following analysis by immunoblot with anti Myc-Tag antibody (Cell signaling). Immunoreactivity was visualized by the Odyssey Imaging System (LICOR Biosciences).

In situ RNAscope analysis. Fresh colon biopsies were collected from the indicated mice and fixed in 4% PFA for 24 hours at 4° C. Samples were then immersed in 10% sucrose at 4° C. for 24 hours, followed by 20% and 30% sucrose for additional 12-24 hours until the tissue sink to the bottom of the 15 ml tube. Samples were then frozen in optimal cutting temperature (OCT) embedding media and store in tissue block container in −80° C. until processing. Seven micrometer thin sections were mount on SuperFrost Plus slides and subjected to single-plex RNAscope 2.5 chromogenic assay BROWN and RNAscope multiplex fluorescent v2 assay according to Advanced Cell Diagnostic (ACD) user manual. Probes were purchased from ACD and are as followed:
Mm-Hoxa11os-O2: target region 575-1839 bp, catalog number 1093151-C1
Mm-mt-Rnr2-C2: target region 25-137 bp, catalog number 590781
Positive control probes were included in the kits: Mm-Ppib-C2: targeting region 98-856 and Mm-Polr2a-C1 targeting region 34-860.

For single-plex RNAscope 2.5 chromogenic assay, slides were stained with 50% Hematoxylin staining solution and mounted using xylene-based mounting medium according to manufacturer's instructions. Samples were visualized using TissueFAXS SL tissue cytometer by UMass Chan medical school SCOPE center (umassmed.edu/SCOPE/).

For RNAscope multiplex fluorescent v2 assay, Mm-Hoxa11os-C1 and Mm-mt-Rnr2-C2 were coupled with Opal™ 520 and Opal™ 690 Reagent Pack (1:1500 dilution), respectively (Akoya bioscience). Samples were then stained with DAPI and mounted with ProLong Gold Antifade Mountant solution. Samples were visualized using Laser scanning confocal microscopy (Leica 8000) at 60× (oil) magnification. Images were taken with 0.2 μm between z-slices in the differential interference contrast. For each biological replicate, at least 8 fields of view were imaged. IMARIS software (Bitplane) was used to analyze the colocalization in three dimensional images. Surface module was exploited to mark mitochondria and Spots module was used to mark HOXA11os. A filter with a negative threshold was used to identify HOXA11os within the mitochondria (beyond the mitochondrial surface). This data was analyzed blindly.

Subcellular fractionation. IECs and LP cells were isolated from the distal colon of WT mice and subjected to cell fractionation assay using RNA subcellular isolation kit (Active Motif). Briefly, 2×10⁶ cells were used for total RNA extraction and 4×10⁶ cells for nuclear/cytoplasm extraction. Cell pellets were resuspended in complete lysis buffer and incubated for 20 minutes on ice. Lysates were centrifuge at 14,000 g for 5 minutes at 4° C. and supernatants were collected as the cytoplasmic fraction. Cell pellets, containing the nuclear fraction, were washed with 70% EtOH and centrifuge at 14,000 g for 5 minutes at 4° C. Supernatants were removed, and Complete buffer G was added to each fraction. Samples were then subjected to column RNA extraction according to the protocol. RNA samples were treated with DNase I to remove genomic DNA and cDNA was synthesized and subjected to RT-qPCR. Expression levels of HOXA11os, GAPDH and 7SK in each fraction were normalized to their expression levels in the input RNA, which was set as 100%.

Mitochondrial Isolation. Mitochondria were purified from colon biopsies collected from the distal colon of WT mice by Mitochondria Isolation kit MITOISO1 (Millipore Sigma). Briefly, mitochondria were isolated from colon biopsies by homogenization in Extraction Buffer A followed by centrifuges according to the manufacturer's instructions. The final mitochondria fraction was resuspended in PBS containing 50 ug/ml RNAse A for 20 minutes at room temperature. Mitochondrial RNA was extracted using Trizol.

ChIRP-MS. LP colonic cells freshly isolated from the distal colons were pooled from 18-23 mice (for each sample) and were subjected to ChIRP-MS analysis as previously described³⁴. An amount of 1×10⁸ cells were crosslinked using 3% FA for 30 min at room temperature following by treatment with 2M glycine for 10 min at room temperature. Cells were then pelleted by spinning at 2000 g for 3 min at 4° C. and lysis for sonication using water bath bioruptor in a 4° C. at highest setting with 30 sec on, 45 sec off pulse intervals. Lysates were then spanned at 16,000 g for 10 min at 4° C. and supernatant was diluted in hybridization buffer. Ten percentages of the lysates were collected as input. Each sample was precleaned with C-1 magnetic beads for 30 min at 37° C. Samples were then collected and 1 μl of 100 μM HOXA11os and control probe mix was added in a ratio of 1 μl probe mix for each 1 ml of sample. Following 4 hr incubation at 37° C., C-1 magnetic beads were added for 30 min incubation at 37° C. Following, beads were washed 5 times and 10% of the resuspended beads were collected for RNA extraction using Trizol. Prior RNA extraction, beads were treated with proteinase K for 45 min at 50° C. followed by 10 min at 95° C. The remaining beads were used for protein extraction in 10% SDS. Samples were subjected to trypsin digestion followed by LC-MS/MS analysis at the UMass Chan Medical School MS core facility.

RNA immunoprecipitation (RIP). RIP was performed using Magna RIP kit (Millipore Sigma) according to the manufacturer's instructions on freshly isolated colonic LP cells isolated from HOXA11os-KO and WT littermate control mice. Antibodies for NDUFV1 and mouse IgG control antibody were used.

Transmission Electron Microscopy. Fresh colon biopsies were collected from the indicated mice and immediately fixed in 2.5% glutaraldehyde/1.6% paraformaldehyde in 0.1 M Sodium Cacodylate buffer pH 7.2, and left overnight at 4° C. The samples were then rinsed three times in the same fixation buffer and post-fixed with 1% osmium tetroxide for 1 h at room temperature before rinsing three times with DH₂O for 10 minutes. samples were then stained with 1% uranyl acetate for 30 mins at RT. and dehydrated through a graded ethanol series (10,30,50,70,85,95%), and 3× 100% ethanol. Samples were then infiltrated first with two changes of 100% Propylene Oxide before leaving overnight in a 50%/50% propylene oxide/SPI-Pon 812 resin mixture. Over the following 2 days seven changes of fresh 100% SPI-Pon 812 resin were done before the samples were polymerized at 68° C. in flat molds. The samples were then reoriented for horizontal sections of the colon tissue layers. The thin sections (approx. 70 nm) were placed on gold support grids, and contrasted with Lead citrate and Uranyl acetate. Sections were examined using the CM10 with 80 Kv accelerating voltage, and images were captured using a Gatan TEM CCD camera.

Oxygen consumption rate (OCR). An amount of 75×10⁵ freshly colonic cells isolated from the distal colon were plated in XF96 cell culture plates in a non-CO₂ incubator. DMEM supplemented with 1 mM pyruvate, 2 mM glutamine and 10 mM glucose in a pH 7.4 was used. OCR was measured under basal conditions using the Seahorse XF extracellular flux analyzer (Agilent).

ATP measurements. An amount of 2×10⁶ freshly IECs and LP cells isolated from the distal colon were lysate in ice cold lysis buffer containing 100 mM Tris, 2M NaCl, 20 mM EDTA and 0.2% Triton X-100. Lysates were then centrifuge for 10 min at 16,000 g and supernatants were collected for the measurement of ATP concentration by ATP determination kit (Molecular Probes A22066) and according to the manufacture's protocol.

NAD+/NADH measurements. An amount of 2×10⁴ freshly IECs and LP cells isolated from the distal colon were lysate in 0.2N NaOH base solution containing 1% DTAB and subjected to NAD+ and NADH measurements by NAD/NADH-Glo™ Assay kit according to manufacturer's protocol (Promega PRG9071).

Mitochondrial ROS measurements. An amount of 5×10⁴ freshly IECs and LP cells isolated from the distal colon were used for the detection of mictochondrial ROS by Mitochondrial ROS detection kit (Cayman chemical 701600). Antimycin A was used as positive control according to manufacturer's protocol.

Mitochondrial DNA measurements. Real time PCR was performed on extracted DNA (QIAGEN) to determine mDNA/nDNA ratio. Primers targeting mitochondrial D-loop region were used to measure mDNA levels. Nuclear DNA levels were measured by primers targeting GAPDH.

Bone marrow chimera. Lethally irradiated (900R) 8-10-week-old recipient male mice were reconstituted by retro-orbital injection of 10⁷ bone marrow cells from age and gender matched donor mice. DSS experiments were performed 8 weeks post reconstitution.

Quantification and Statistical Analysis. Samples sizes were chosen with adequate statistical power based on previously published comparable studies. Differences between group means were tested with a t-test when the experiment contained two groups, or one-way ANOVA (followed by a Tukey post hoc test) when the experiment contained more than two groups. In cases where the experiment was running in two batches, “batch” was entered as a random categorical factor into a 2-way ANOVA. All pathology evaluation and takedowns were performed blindly. Data are presented as mean±SEM. For in vivo and in vitro experiments, n represent number of animal or number of cells, respectively. Values of p<0.05 were considered statistically significant. *P<0.05, **P<0.01, ***P<0.001.

For microbiome sequencing, ecological diversity metrics were generated using QIIME2 software version 2020.2⁷³. Beta diversity significance was tested using pairwise PERMANOVA using Bray-Curtis dissimilarity. Differential abundance of taxa was evaluated using MaAsLin2 version 0.99.12 (Multivariate Analysis by Linear Models 2; huttenhower.sph.harvard.edu/maaslin2).

### Example 1. Murine lncRNA HOXA11Os and the Human Ortholog HOXA11AS are Uniquely Expressed in the Distal Colon and Downregulated During Intestinal Inflammation

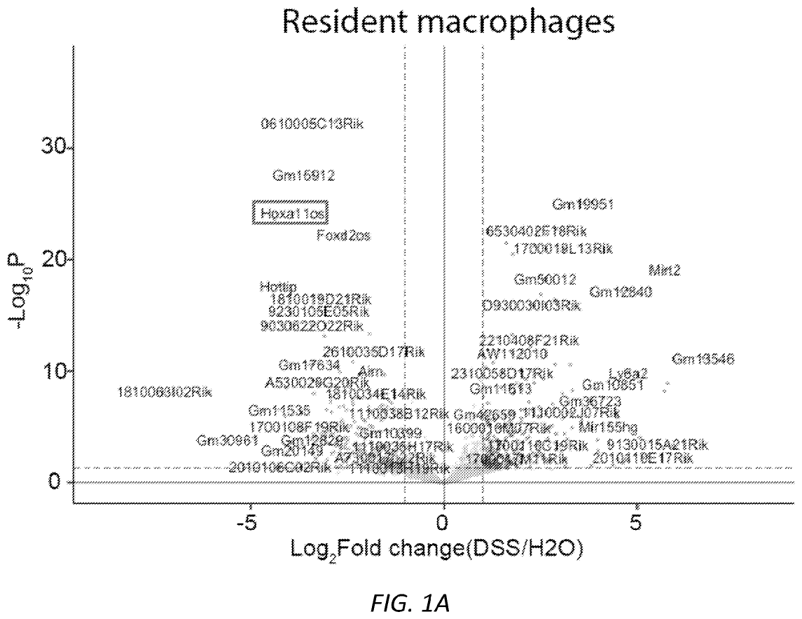

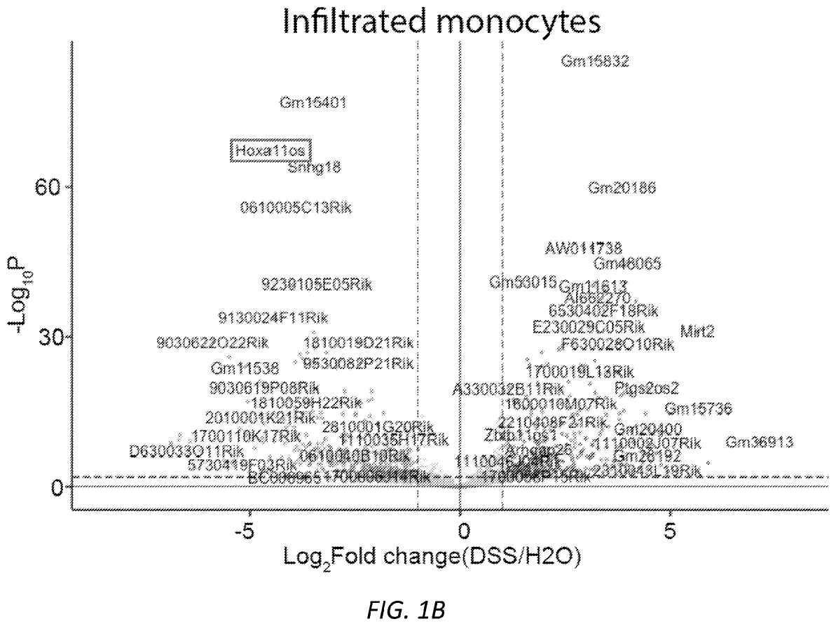

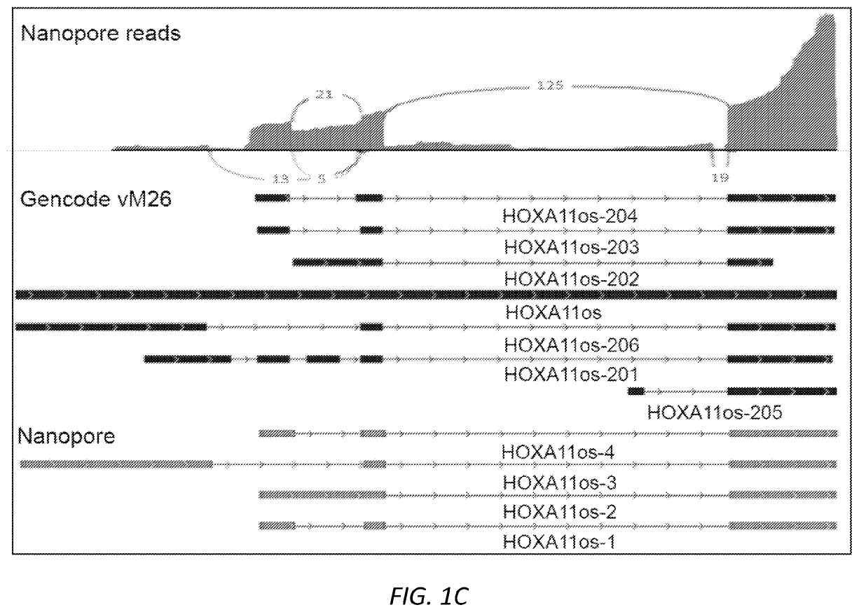

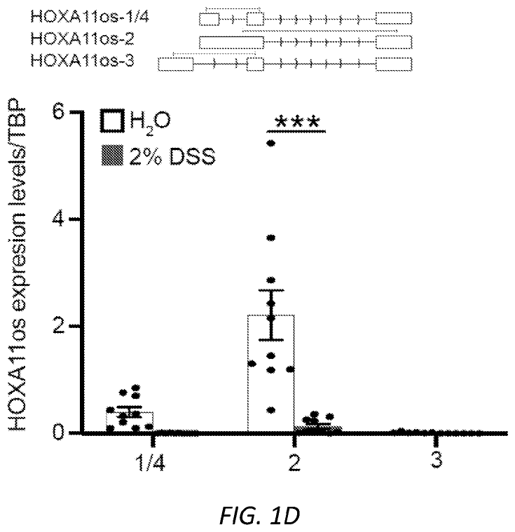

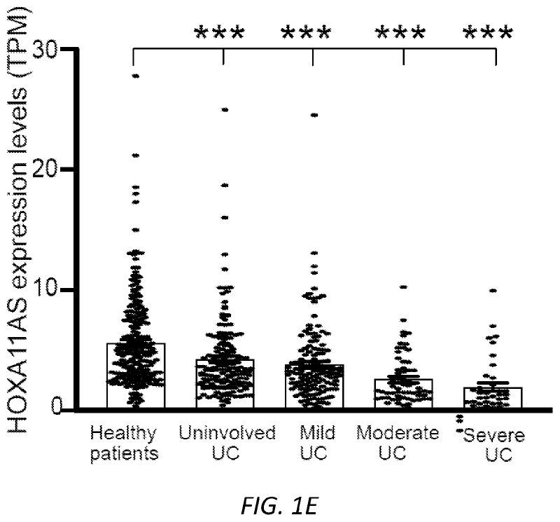

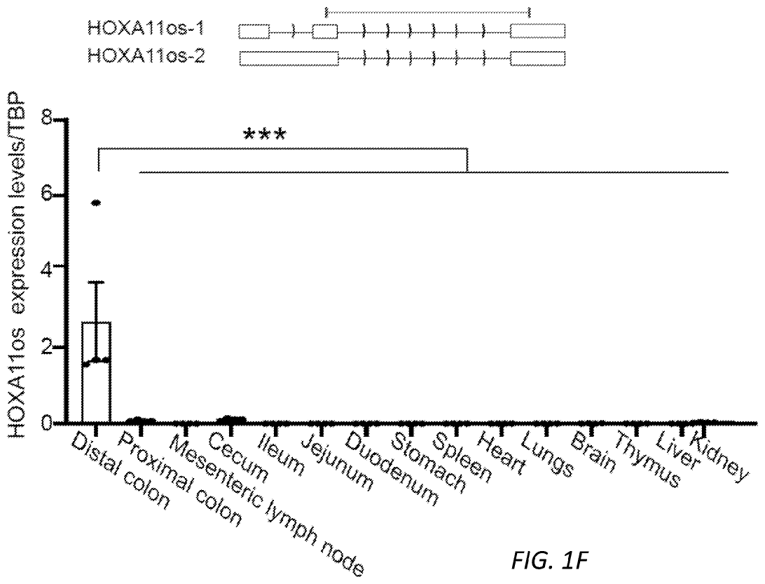

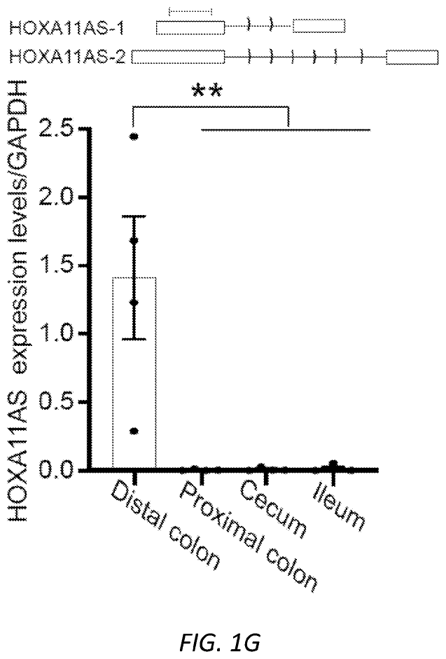

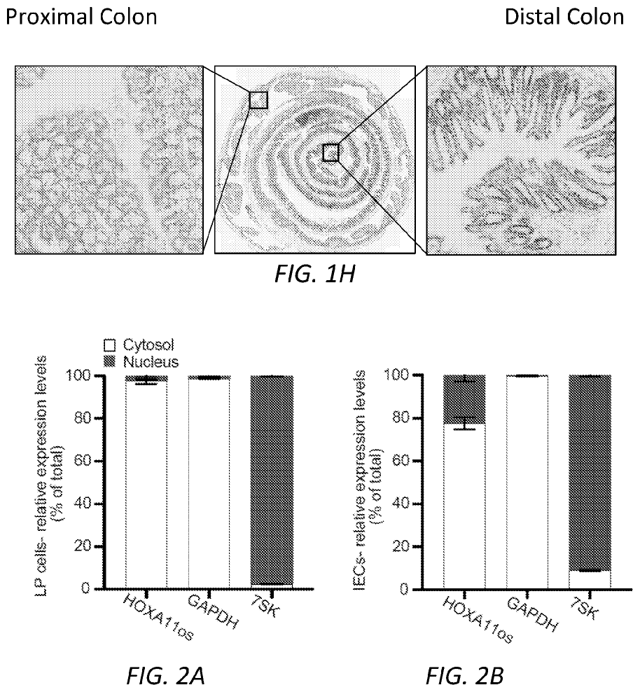

To identify lncRNAs with regulatory function in UC, we performed transcriptome analysis of resident macrophages and infiltrated monocytes in the colon of mice that were either untreated or treated with dextran sodium sulfate (DSS) for 7 days to induce acute colitis 24. LncRNAs were differentially expressed in these cells under inflammatory conditions (FIG. 1A-B). Immune genes (protein coding) in these cells were also differentially regulated, as expected. Given the large number of differentially expressed lncRNAs in the colon, we prioritized lncRNAs that were highly abundant in the colon, differentially expressed in a manner that correlates with colitis severity and conserved in humans. A lncRNA in the HOX locus, Homeobox A11 opposite strand (HOXA11os) was amongst the most abundant lncRNAs in the healthy colon. HOXA11os was significantly downregulated under conditions of intestinal inflammation (FIG. 1A-B), suggesting a potential regulatory role for HOXA11os during intestinal homeostasis.

Unlike protein coding genes, lncRNAs are poorly annotated due to a lack of hallmark characteristics such as transcription initiation and termination. LncRNAs also undergo extensive alternative splicing, which increases the potential number of isoforms ²⁵. It is, therefore, essential to identify the HOXA11os transcripts expressed in the colon. To this end, we utilized long read sequencing (Oxford Nanopore), a next generation sequencing method which allows for real time analysis of long RNA fragments and the identification of previously unannotated transcripts. Nanopore sequencing identified 4 independent transcripts of HOXA11os in the healthy colon of wild-type (WT C57BL/6) mice (FIG. 1C, Table 1). Transcripts 1 and 4 displayed high sequence similarity with minor differences in the second exon. Transcript 4 had a small number of splicing events relative to transcript 1 (FIG. 1C), suggesting transcript 1 of HOXA11os is more abundant. To quantify the expression levels of these isoforms, we used RT-qPCR with exon spanning primers to detect the mature/spliced form of each transcript (FIG. 1D). This analysis indicated that while transcript 3 was undetectable, transcript 2 was the most abundant transcript in the colon and this transcript was significantly downregulated in colitic mice (FIG. 1D). Thus, transcript 2, a novel isoform of HOXA11os is abundant in the healthy colon.

We next analysed a large human IBD cohort from the Mount Sinai Crohn's and Colitis Registry (MDCCR) which included RNA-sequencing of colon biopsies from more than a thousand patients ^{26, 27}. As in the mouse, the human ortholog, homeobox A11 antisense (HOXA11AS) was abundant in colon biopsies from healthy patients and significantly reduced in patients with UC (FIG. 1E). The levels of HOXA11AS were decreased in a manner that correlated with disease severity (FIG. 1E). Interestingly, the levels of HOXA11AS were higher in adjacent non-inflamed areas of the colon in these patients (FIG. 1E).

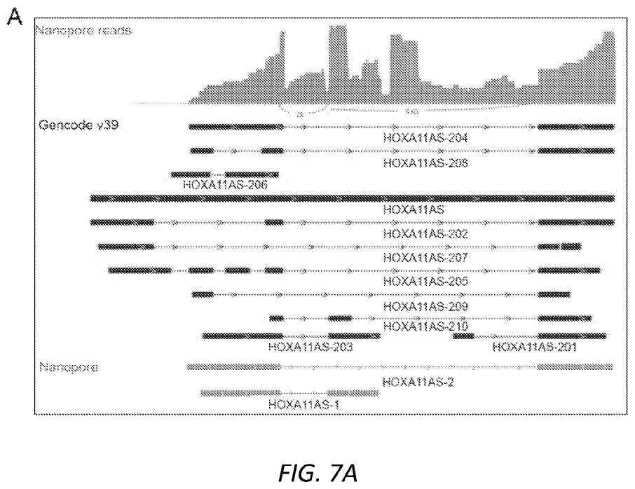

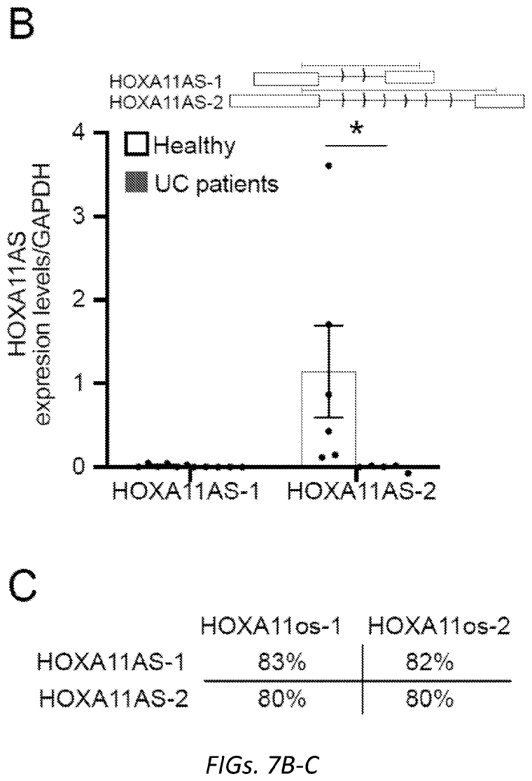

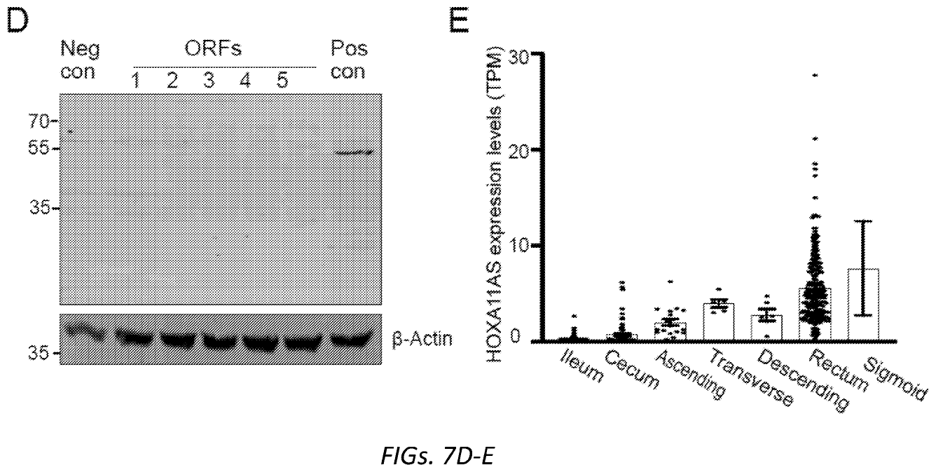

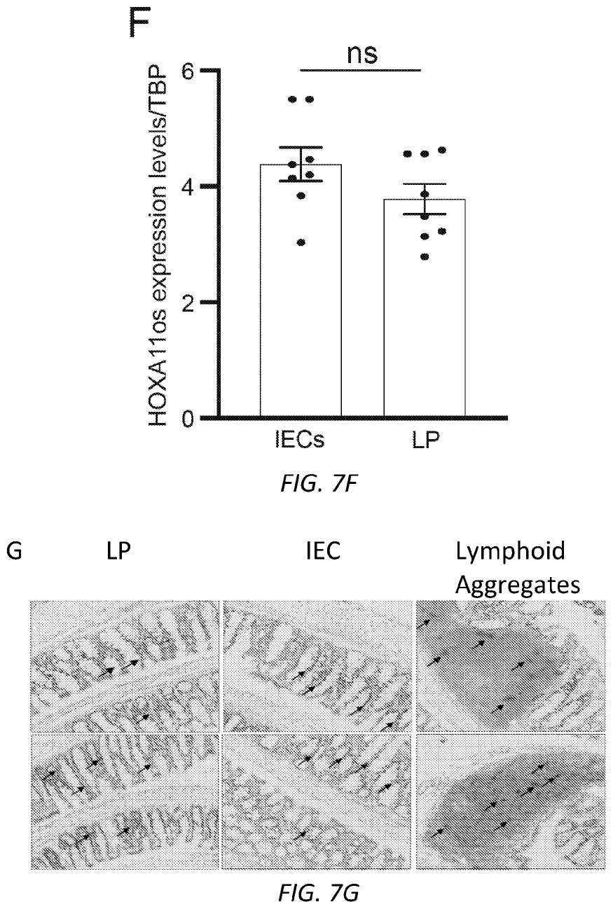

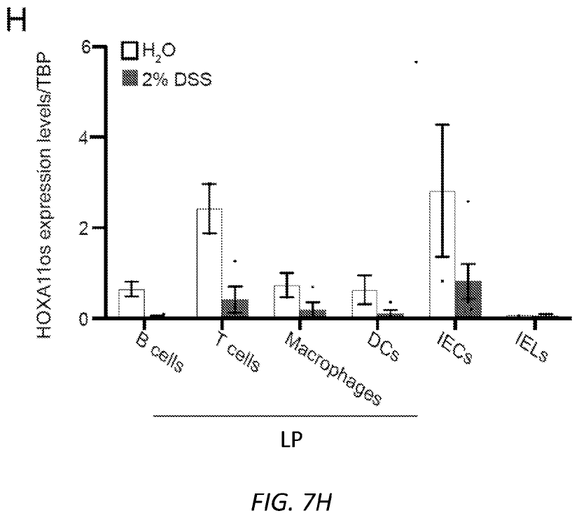

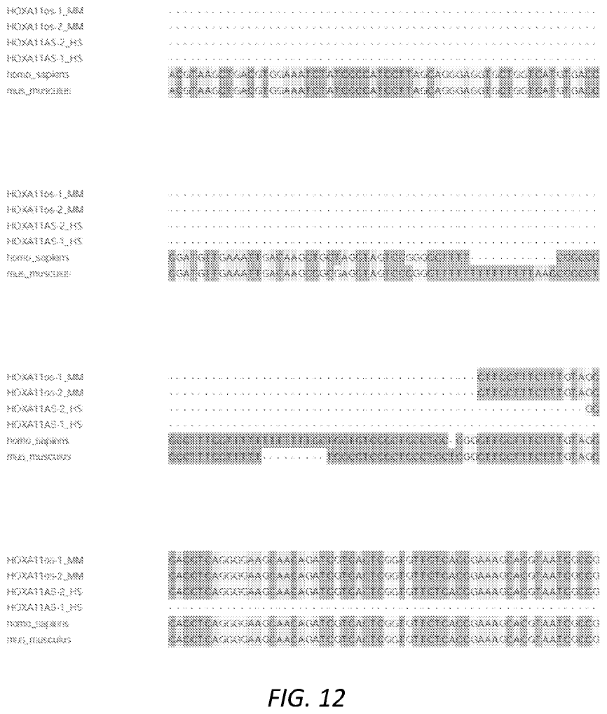

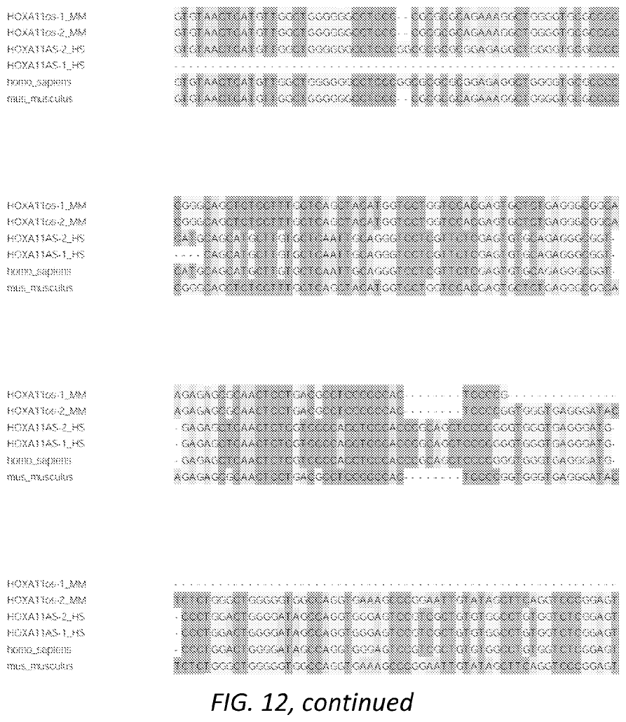

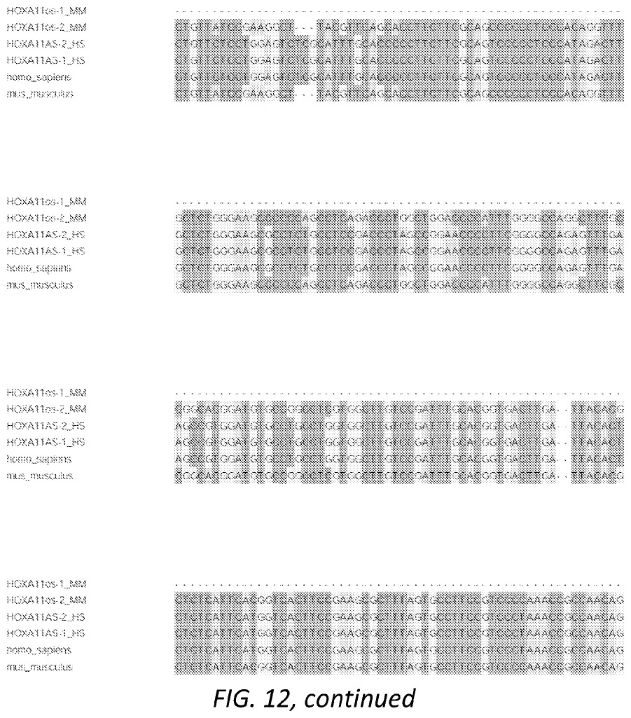

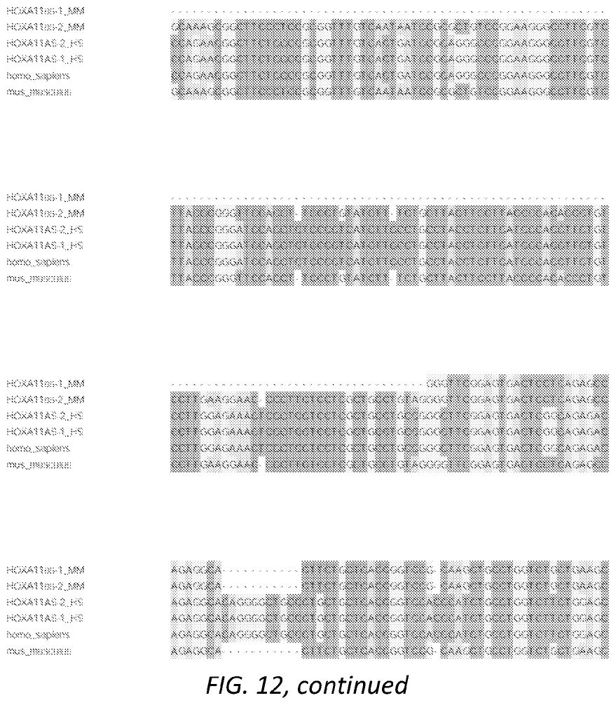

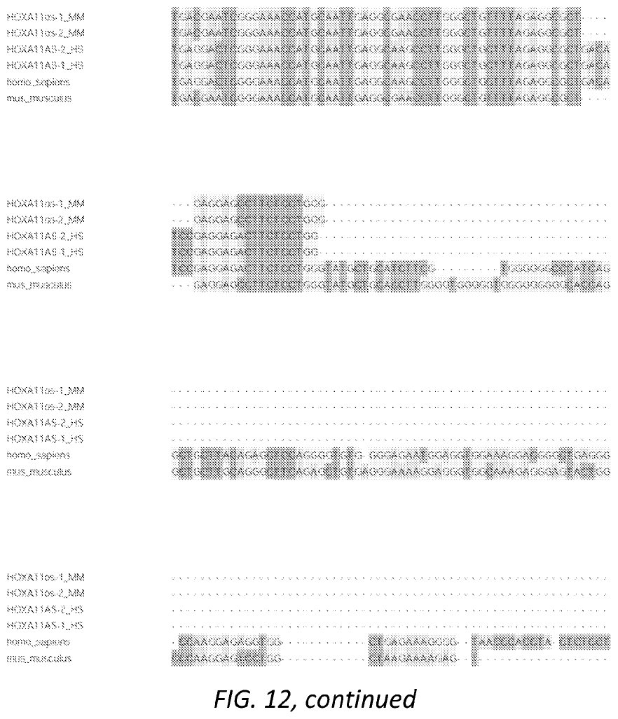

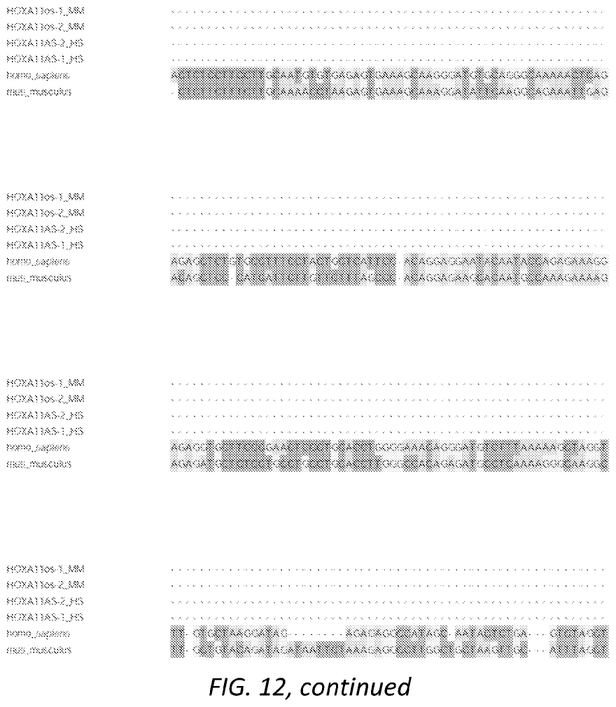

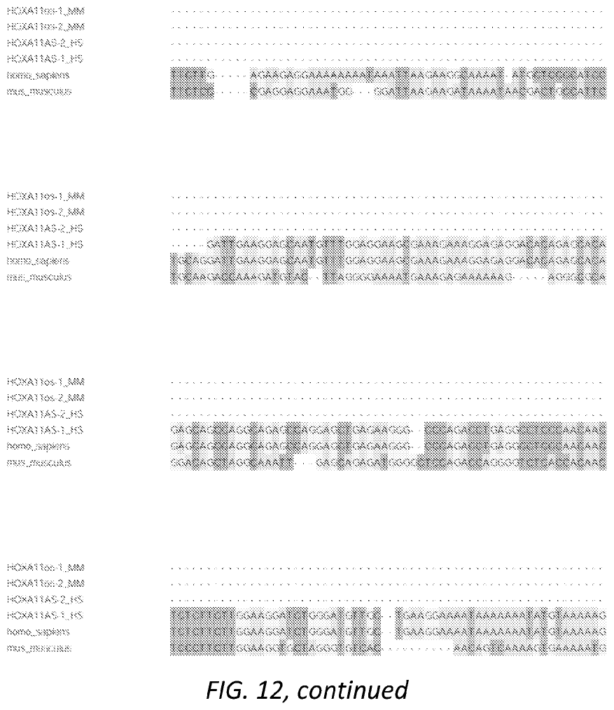

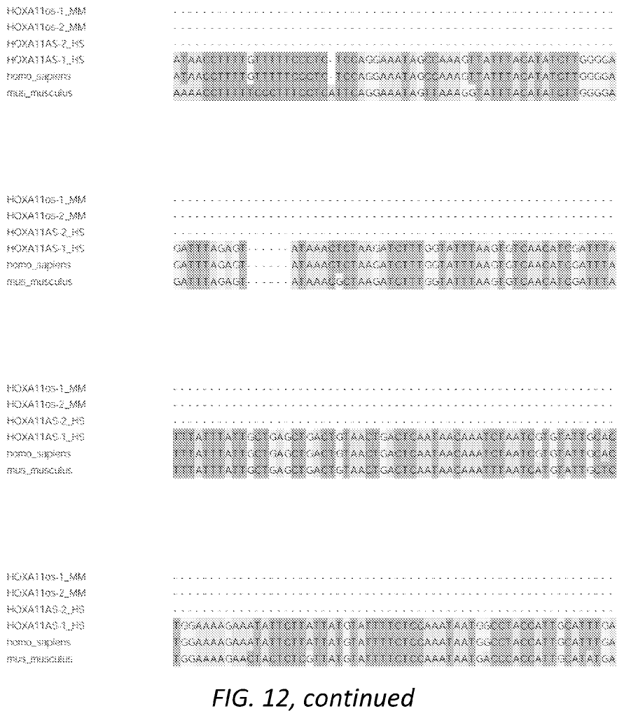

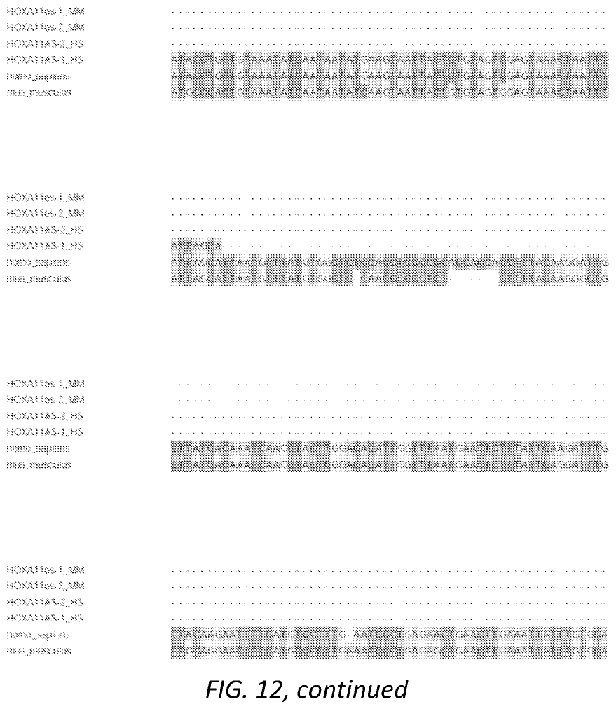

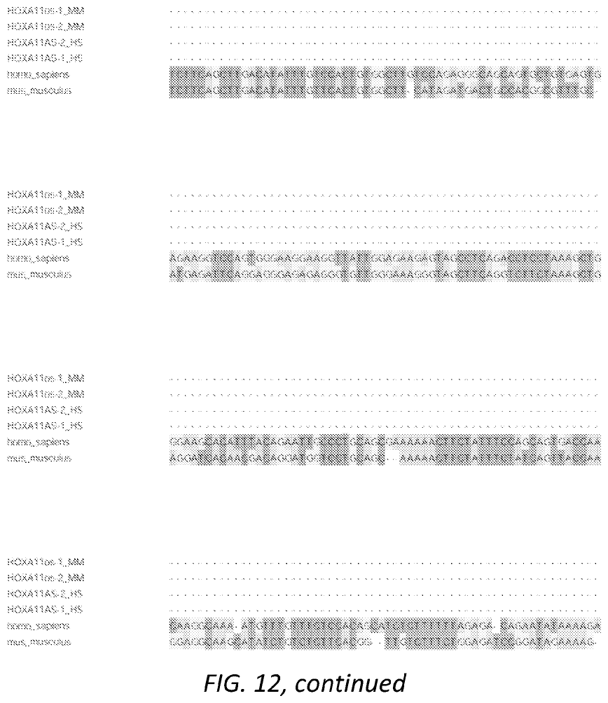

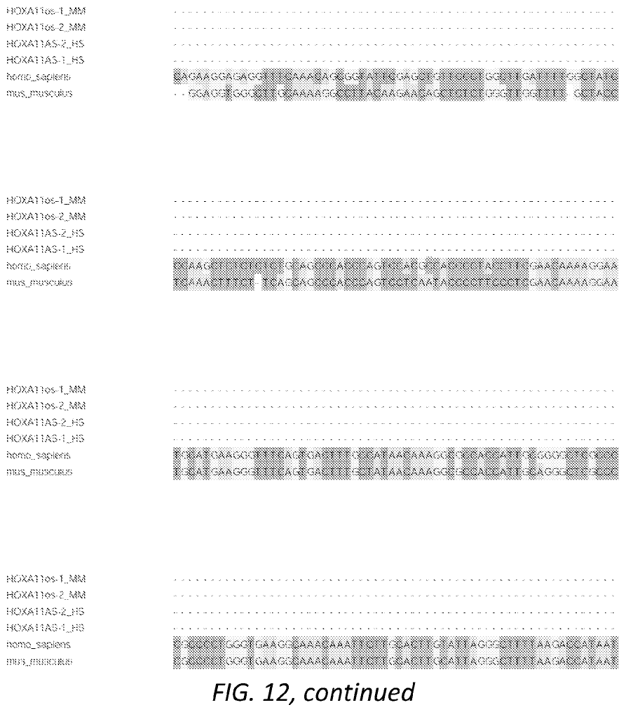

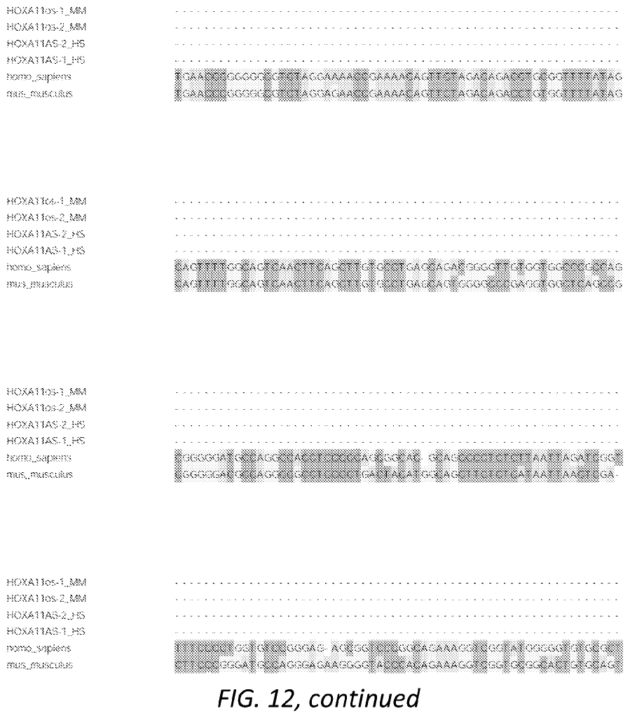

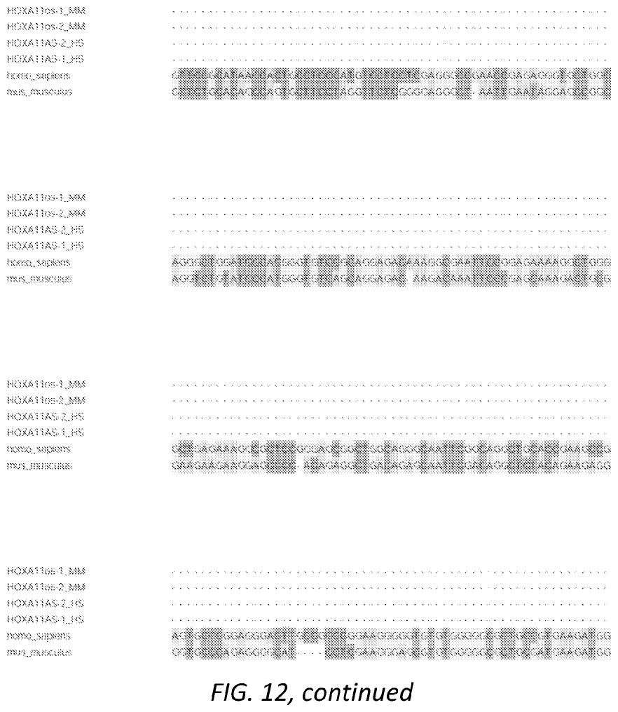

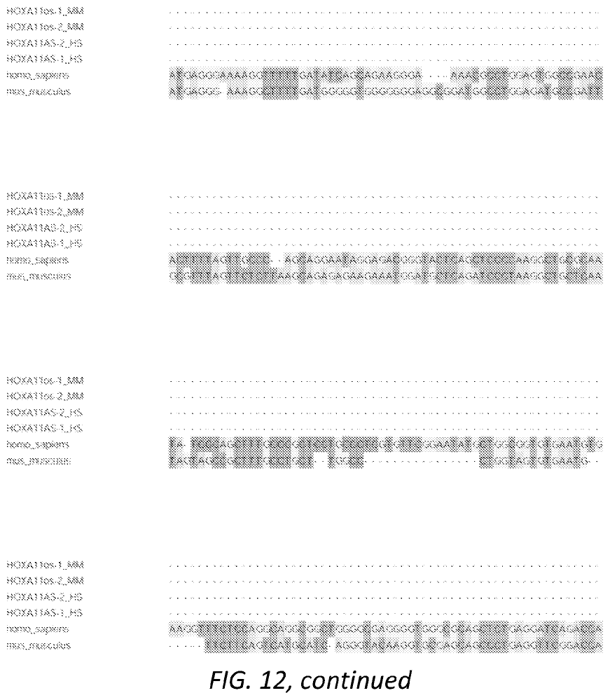

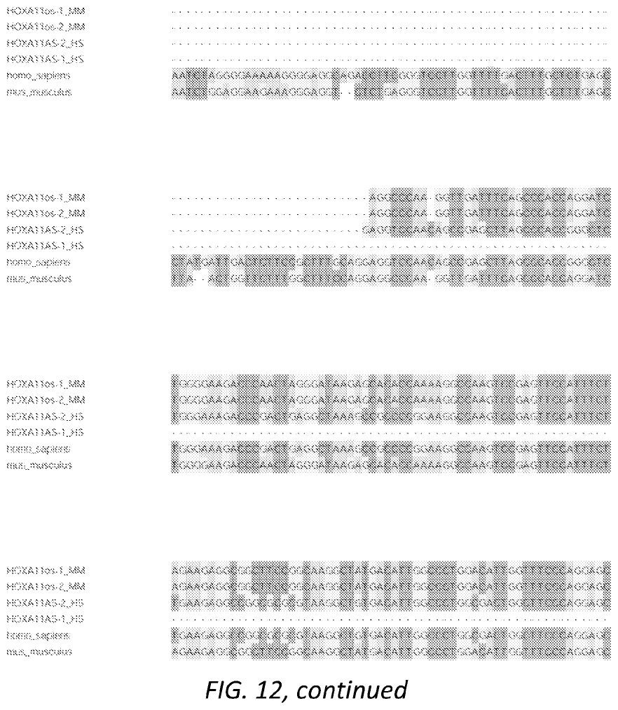

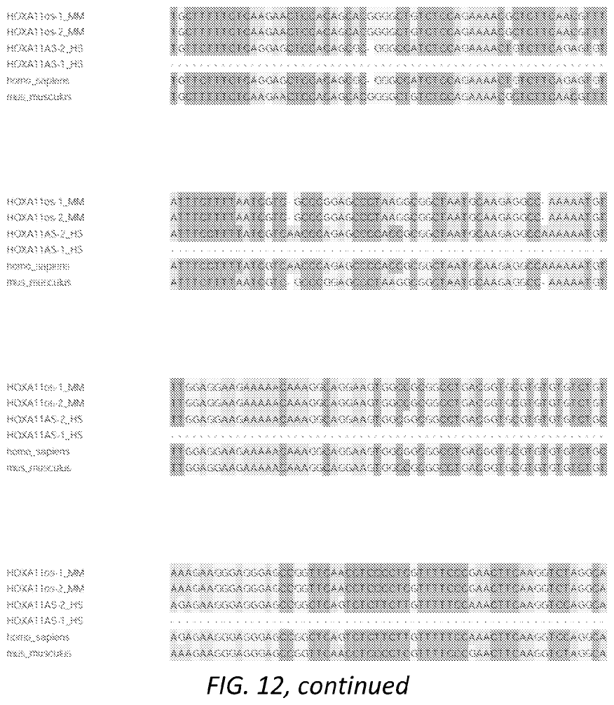

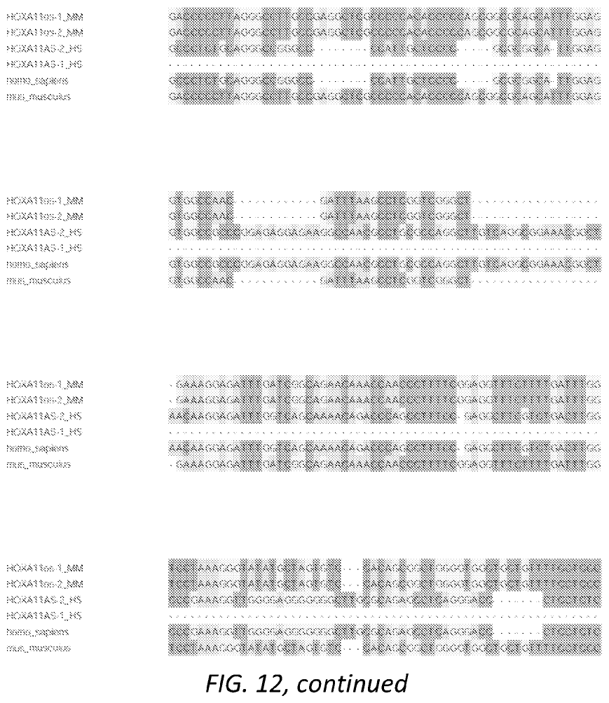

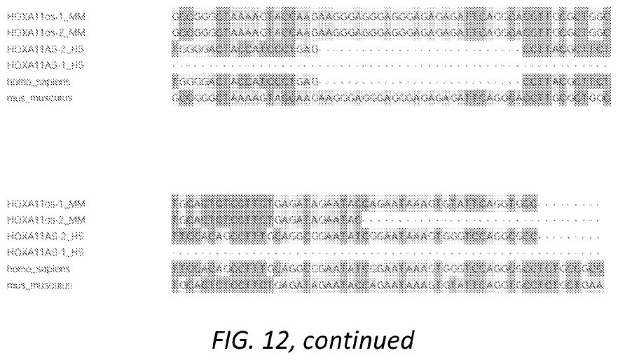

We collected fresh colon biopsies from healthy human control patients and performed long read sequencing, which identified 2 transcripts of HOXA11AS in the human colon (FIG. 7A, Table 2, SEQ ID NOs: 5 and 6). RT-qPCR using exon spanning primers showed that transcript 2 of HOXA11AS was the most abundant transcript in the healthy colon and this was reduced in the inflamed colon of patients with UC (FIG. 7B). Murine HOXA11os on chromosome 6 and human HOXA11AS on chromosome 7 are highly conserved lncRNAs with 80% identity as calculated by PFAAT (FIG. 7C, FIG. 12)²⁸.

We next wanted to determine if there was any coding potential within the HOXA11os sequence. Through NCBI ORF finder, we identified 5 putative ORFs (Table 3). To test the coding potential of these putative ORFs, we cloned and transfected the myc-tagged ORFs into HEK293T cells. Western blotting revealed that the HOXA11os ORFs were not translated (FIG. 7D). Thus, HOXA11os appears to lack coding potential.

### Example 2. Murine HOXA11Os and Human HOXA11AS Expression is Restricted to the Distal Colon

LncRNAs are expressed in a more tissue restricted manner than protein coding genes, highlighting potential physiological functions in the tissues or cell types in which they are expressed ^{1,29}. HOXA11os expression was restricted to the distal colon of mice (FIG. 1F). In human tissue HOXA11AS demonstrated a similar expression pattern and was predominantly expressed in the distal colon when assessed across the intestinal tract (FIG. 1G, FIG. 7E) ^{26,27}. HOXA11os was expressed at high levels in CD45+ cells of the lamina propria (LP) and CD45⁻ epithelial cells (IECs) (FIG. 7F). To further assess the expression pattern of endogenous HOXA11os in colonic tissue, we utilized chromogenic RNA in situ hybridization (IHC), RNAscope, which enables the visualization of RNA at single molecule resolution in the intact colon (FIG. 1H, FIG. 7G). RNAscope analysis confirmed that HOXA11os was highly abundant in the distal colon and undetectable in the proximal colon (FIG. 1H). Within the distal colon, HOXA11os was expressed at high levels in IECs, LP and lymphoid aggregates (FIG. 7G). We also quantified HOXA11os expression in isolated cells from the distal colon. HOXA11os was expressed at high levels in both IECs and LP resident immune cells and was downregulated during colitis (FIG. 7H). HOXA11os was undetectable in intraepithelial lymphocytes (IELs) under basal conditions (FIG. 7H). These observations indicate that HOXA11os is a conserved lncRNA expressed in both hematopoietic and non-hematopoietic cells of the distal colon.

### Example 3. HOXA11Os is Localized to the Mitochondria and Interacts with Mitochondria Associated Proteins

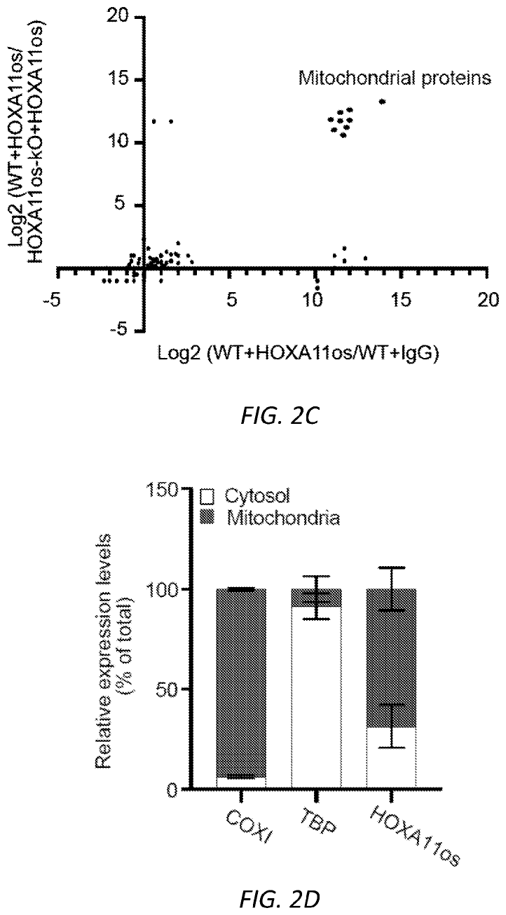

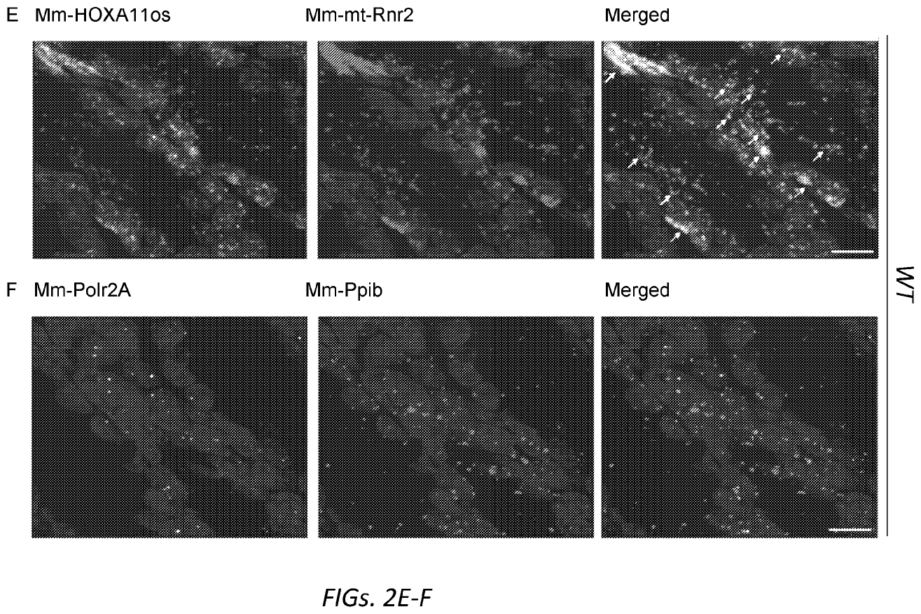

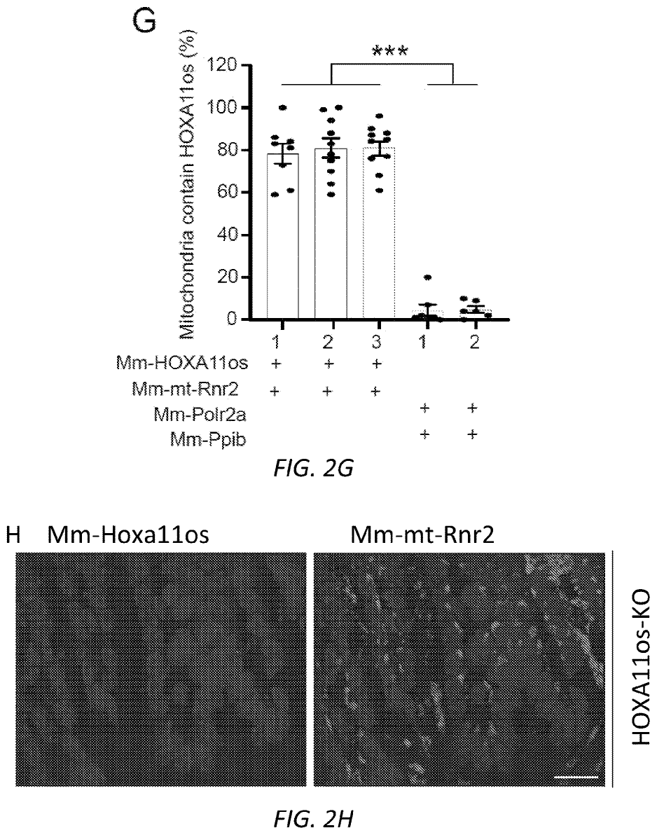

The functionality of lncRNAs depends on their sub-cellular localization 30. Nuclear localized lncRNAs have been reported to regulate chromatin structure, transcription, RNA splicing, RNA stability and nuclear condensates. Whereas cytoplasmic localized lncRNAs impact mRNA stability, translation, protein localization and protein turnover. Cytoplasmic lncRNAs also regulate protein function by scaffolding of proteins operating in shared signaling pathways ^{31, 32}. We therefore characterized the subcellular localization of HOXA11os in colonic LP cells (FIG. 2A) or IECs (FIG. 2B). As expected, the mature GAPDH mRNA transcript was enriched in cytosolic fractions (FIG. 2A-B), while 7SK (a nuclear snRNA) was enriched in the nuclear fractions (FIG. 2A-B). Mature HOXA11os was enriched in the cytosolic fraction in both LP cells and IECs (FIG. 2A-B).

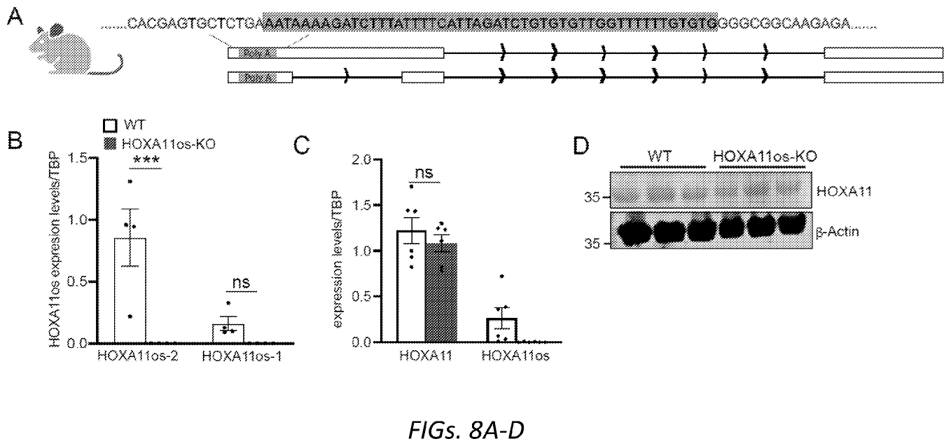

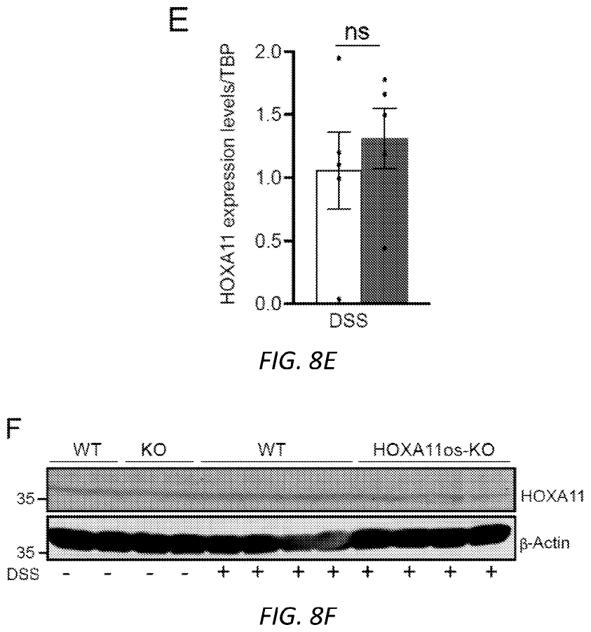

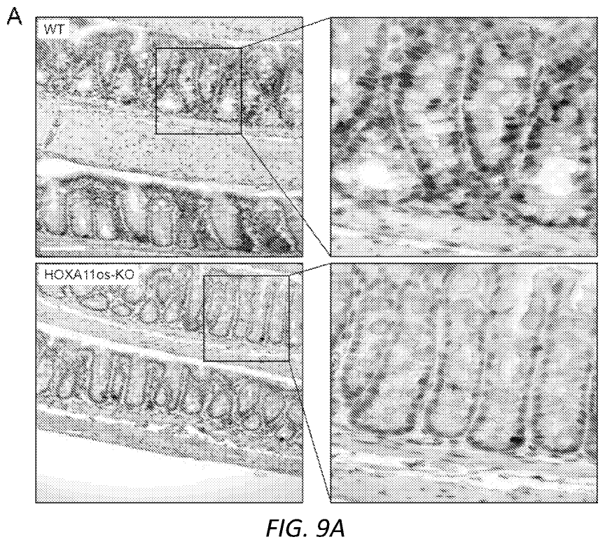

We next generated mice lacking HOXA11os through an innovative CRISPR/Cas9 approach that introduced a short 49-bp synthetic polyadenylation signal (spA) ³³ in exon 1 proximal to the transcription start site of HOXA11os (FIG. 8A). This resulted in a complete loss of HOXA11os transcripts 1 and 2 (FIG. 8B) with no effect on the expression of the proximal protein coding gene HOXA11 under basal (FIG. 8C-D) or colitis (FIG. 8E-F) conditions.

We therefore sought to identify the protein binding partners of HOXA11os. To this end, HOXA11os-KO and littermate control mice were subjected to comprehensive identification of RNA-binding proteins by mass spectrometry (ChIRP-MS) ³⁴ (FIG. 2C). An amount of 1×10⁸ primary colonic LP cells were collected from the distal colon of 18-23 pooled HOXA11os-KO or WT littermate control mice. The cells were then crosslinked and incubated with biotinylated antisense DNA probes designed to be complementary to the HOXA11os transcript and tiled along the full length of the transcript (Table 4). Probes targeting the Drosophila ROX gene were used as an irrelevant control. Probes were then captured by streptavidin magnetic beads followed by elution of RNA and proteins which were subsequently identified by liquid chromatography-mass spectrometry (LC-MS). Specific enrichment of endogenous HOXA11os complexes using HOXA11os specific probes was quantified by RT-qPCR. There was no enrichment of HOXA11os when lysates from HOXA11os-KO mice were incubated with specific probes, or when ROX probes were used. Further, GAPDH mRNA was undetectable following HOXA11os pulldown, further confirming the specificity of HOXA11os antisense purification. We then subjected these pulldowns to mass spectrometry to identify HOXA11os-associated proteins. Remarkably, HOXA11os interacted with a number of enzymes with shared functions of metabolic pathways in the mitochondria (FIG. 2C). Indeed, HOXA11os interacted with mitochondrial proteins associated with the Krebs cycle, ETC and OXPHOS. HOXA11os interacting proteins included a subunit of the mitochondrial membrane respiratory chain complex I (NADH dehydrogenase flavoprotein 1), proteins associated with fatty-acid oxidation and the Krebs cycle associated proteins Succinate coenzyme A (coA) ligase [ADP forming] subunit beta³⁵, as well as Very long-chain specific acyl-CoA dehydrogenase (ACADVL), Isoform 2 of Acyl-coenzyme A synthetase (ACSM3), Hydroxymethylglutaryl-CoA lyase (HMGCL), and Heat shock protein 75 kDa (TRAP1).

Recent studies have indicated that certain lncRNAs can localize to specific subcellular organelles ³⁰ and regulate their activity ³⁶⁻³⁸. Given that HOXA11os interacts with proteins localized in the mitochondria, all of which are part of shared metabolic pathways, we next evaluated the sub-cellular localization of HOXA11os. To this end, mitochondrial and cytoplasmic fractions were purified from distal colon tissue of WT mice and interrogated by qRT-PCR. As expected, the mRNA transcript of the mitochondrial encoded protein COXII was enriched in the mitochondrial fraction (FIG. 2D), while the mRNA transcript of the nuclear encoded protein TBP (TATA-Binding Protein) was enriched in cytoplasmic fractions (FIG. 2D). Mature HOXA11os was also enriched in the mitochondrial fraction (FIG. 2D). These data were further confirmed by RNAscope (FIG. 2E-H). HOXA11os and mitochondria were co-localized in biopsies collected from the distal colon of WT mice (FIG. 2E). DAPI was used to stain cell nuclei. As a control, RNA polymerase ll subunit A and Peptidylprolyl Isomerase B were also monitored (FIG. 2F). Strikingly, we found that approximately 80% of mitochondria of the distal colon were highly enriched for HOXA11os (FIG. 2G). No co-localization was observed between the control probes detecting RNA polymerase II subunit A (FIG. 2G). HOXA11os was also detected at low levels in the nucleolus (FIG. 2E). The nuclear signal of HOXA11os likely represents newly transcribed, immature transcripts of HOXA11os. HOXA11os was undetectable in the colon of HOXA11os-KO mice underscoring the specificity of the probes used (FIG. 2H).

### Example 4. HOXA11Os-KO Mice Exhibit Impaired OXPHOS and Age-Dependent Spontaneous Intestinal Inflammation

OXPHOS is a cellular process that harnesses the reduction of oxygen to generate phosphate bonds in the form of adenosine triphosphate (ATP) from adenosine diphosphate (ADP). Defects in OXPHOS have been associated with intestinal inflammation ³⁹⁻⁴¹. Given that HOXA11os is localized to mitochondria and interacts with proteins coupled to mitochondrial OXPHOS, we next assessed whether deficiency in HOXA11os altered OXPHOS. Mitochondrial OXPHOS can be measured by monitoring the mitochondrial oxygen consumption rate (OCR). Colonic cells isolated from the distal colon of HOXA11os-KO mice under basal conditions had lower OCR levels when compared to cells from WT littermate control mice (FIG. 3A), suggesting that HOXA11os deficiency results in a lower basal respiratory rate.

HOXA11os deficient mice exhibited spontaneous intestinal inflammation as they aged, characterized by elevated levels of the pro-inflammatory cytokines IL-6 (FIG. 3B), IL-1β (FIG. 3C) and RANTES (FIG. 3D) at 4-months of age when compared to WT littermate controls. HOXA11os deficient mice displayed diarrhea and blood in their feces at 4-months of age. However, body weights and colon length were comparable between HOXA11os-KO and their WT littermate controls. Histological features, immune cell composition and IEC composition were all comparable between HOXA11os-KO and WT littermate controls.

### Example 5. HOXA11Os Deficient Mice are Highly Susceptible to Experimental Models of Colitis

Given that HOXA11os deficiency predisposed animals to spontaneous inflammation and impaired OXPHOS in the colon, we hypothesized that HOXA11os was protective for colitis. HOXA11os-KO and WT littermate control mice were subjected to acute colitis using dextran sulfate sodium (DSS) administered in the drinking water for 7 days (FIG. 3E). HOXA11os-KO mice were more susceptible to intestinal inflammation when compared to co-housed WT littermate controls (FIG. 3F-Q), as demonstrated by shorter colon length (FIG. 3F,J), increased weight loss (FIG. 3G,K), higher score for stool appearance (FIG. 3H-L) and higher pathological scores (FIG. 3I,M-N). This enhanced susceptibility to colitis was observed with both male and female animals (FIG. 3F-M). HOXA11os-KO mice treated with DSS also had higher levels of pro-inflammatory cytokines (FIG. 3O-Q) and Interferon stimulated genes (ISGs) when compared to WT littermate control mice. Thus, HOXA11os deficiency sensitizes mice to colitis.

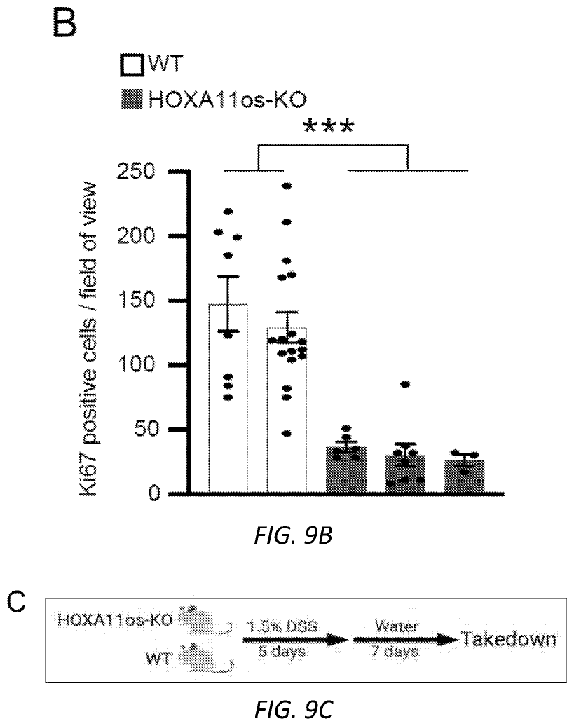

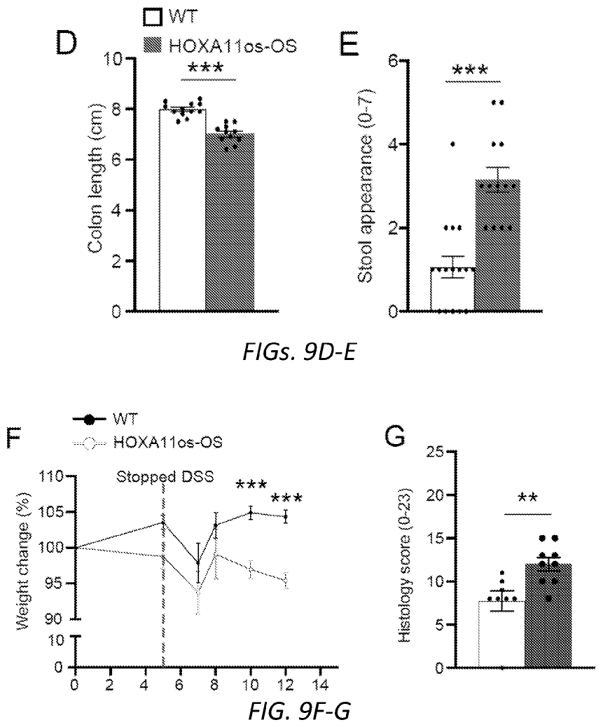

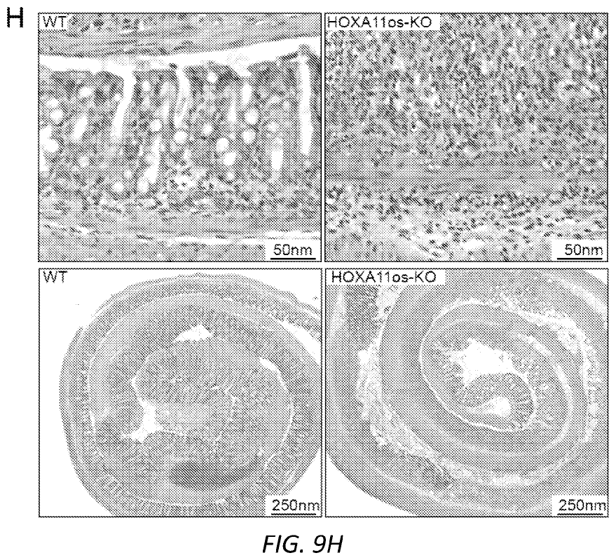

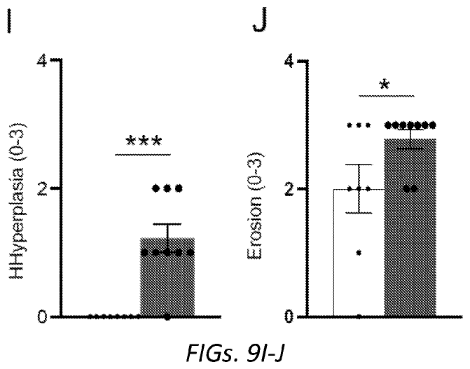

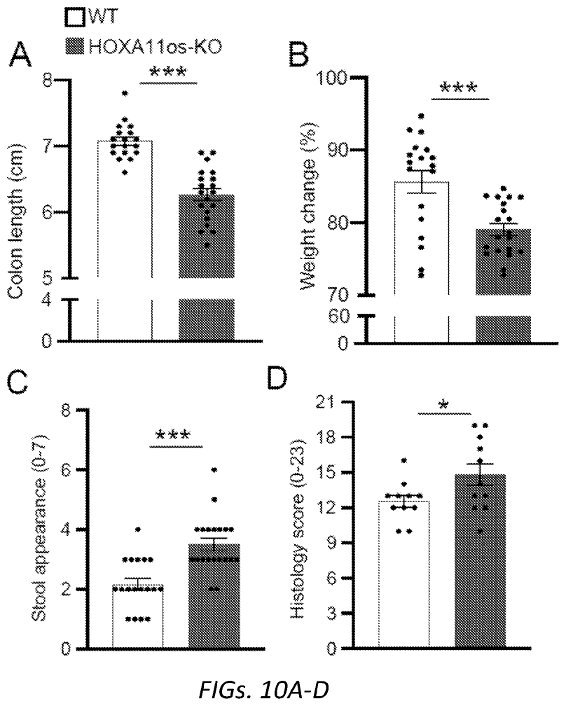

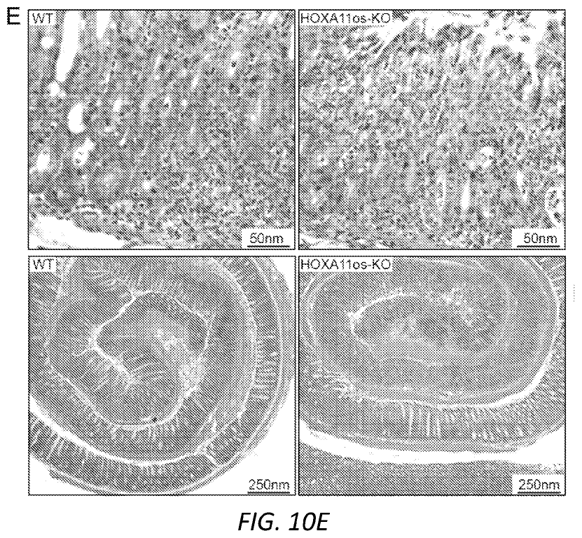

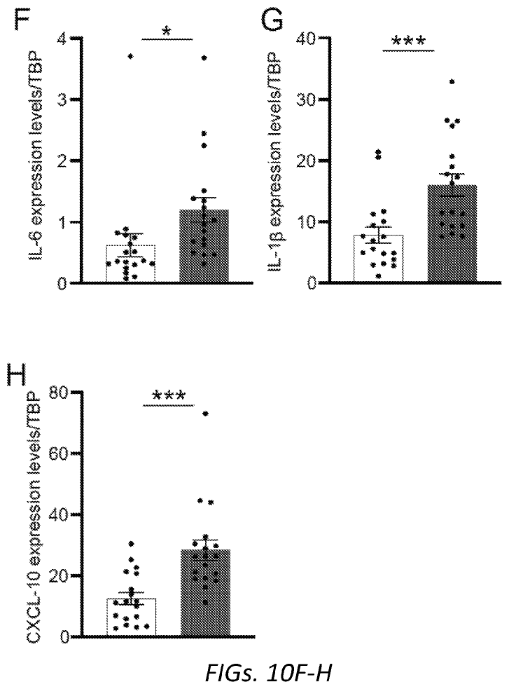

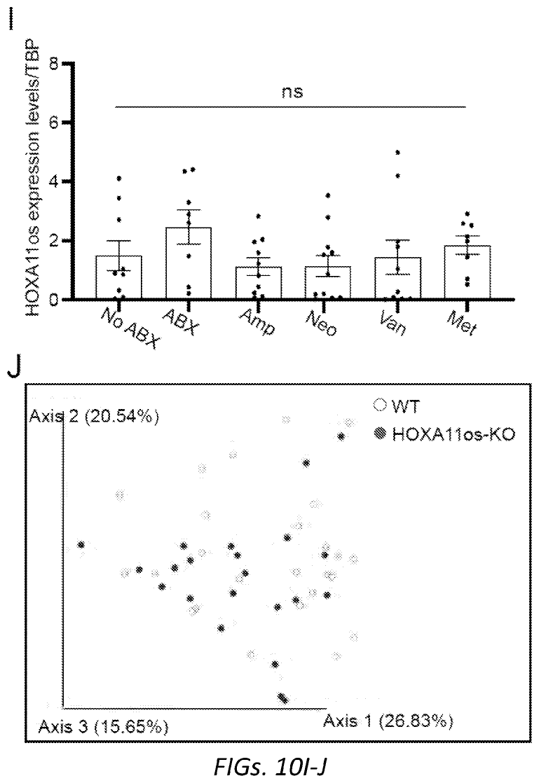

HOXA11os-KO mice also had a reduction in the level of the proliferative marker, Ki67, in the distal colon when compared to WT littermate control mice, suggesting a failure to restore barrier integrity (FIG. 9A-B). We therefore subjected these mice to a recovery model of DSS-induced colitis to assess the role of HOXA11os in the recovery phase of intestinal inflammation. HOXA11os-KO and WT littermate control mice were treated with DSS for 5 days followed by a 7-day recovery period on regular water (FIG. 9C). HOXA11os-KO mice continued to exhibit severe colitis during the recovery phase as demonstrated by shorter colon lengths (FIG. 9D), diarrhea and traces of blood in the feces (FIG. 9E), increased weight loss (FIG. 9F) and higher pathological scores (FIG. 9G-J). In contrast, littermate control mice recovered and had restored intestinal integrity (FIG. 9D-J). In addition to DSS-induced colitis, HOXA11os-KO mice challenged with Salmonella enterica serovar Typhimurium SL1344 displayed shorter colon lengths (FIG. 10A), greater weight loss (FIG. 10B), higher colitis scoring (FIG. 10C), higher pathological scores (FIG. 10D-E) and elevated levels of the pro-inflammatory cytokines IL-6 (FIG. 10F), IL1-1β (FIG. 10G) and CXCL-10 (FIG. 10H) when compared to co-housed WT littermate controls.

We next characterized the contribution of the microbiome to the expression of HOXA11os in the distal colon using antibiotic-induced microbiota depletion (FIG. 10I). Following administration of a broad-spectrum antibiotic cocktail (containing neomycin, ampicillin, vancomycin and metronidazole) for 4 weeks which resulted in a complete depletion of the microbiota, WT mice expressed comparable levels of HOXA11os in the distal colon as those treated with regular water (FIG. 101). Mice were also administrated individual antibiotics to deplete specific bacterial families. WT mice treated with neomycin (largely gram-negative targeting), ampicillin (broad-spectrum targeting), vancomycin (largely gram-positive targeting) and metronidazole (largely anaerobic targeting) displayed similar expression levels of HOXA11os as those treated with regular water (FIG. 101). These data suggest that HOXA11os expression is not dependent on the microbiota. We also assessed whether HOXA11os deficiency altered the composition of the microbiota in the colon, thus leading to increased susceptibility to acute colitis. Shotgun sequencing of stool samples collected from HOXA11os-KO and WT littermate control mice demonstrated that HOXA11os deficiency had no effect on the colonialization of microbiota in the intestine under homeostatic conditions (FIG. 10J).

### Example 6. Deficiency of HOXA11Os in Myeloid Cells Promotes Colitis

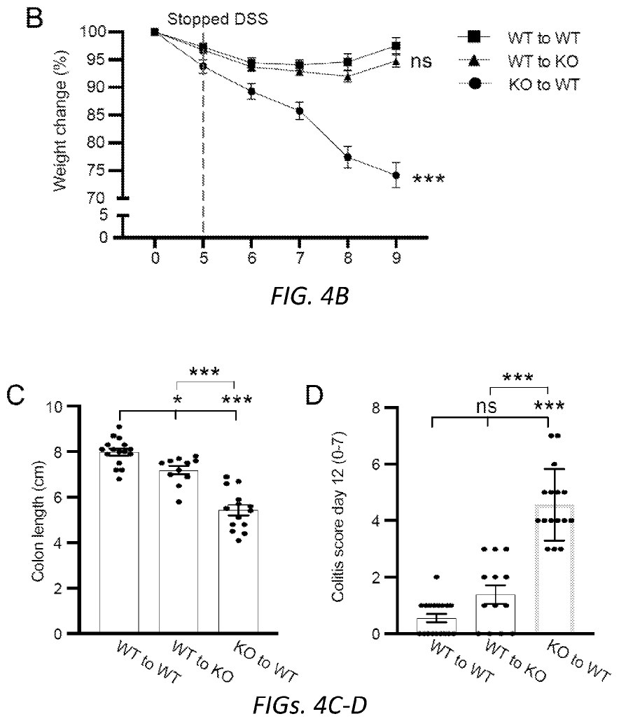

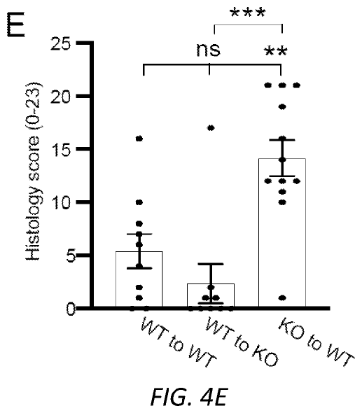

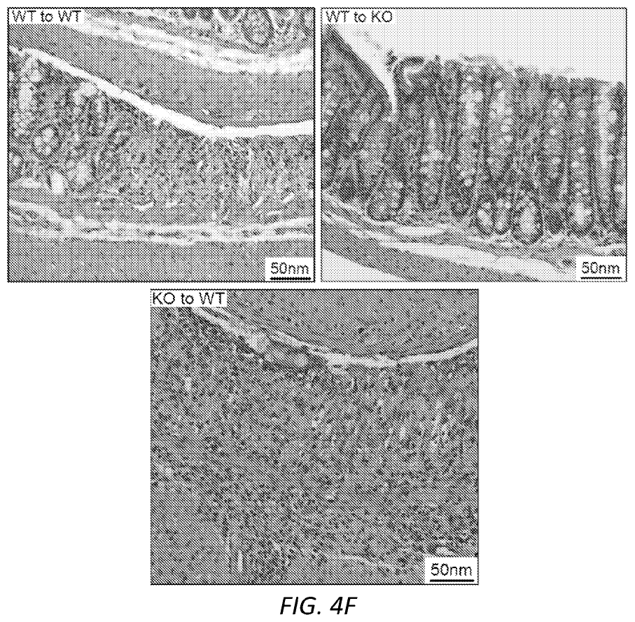

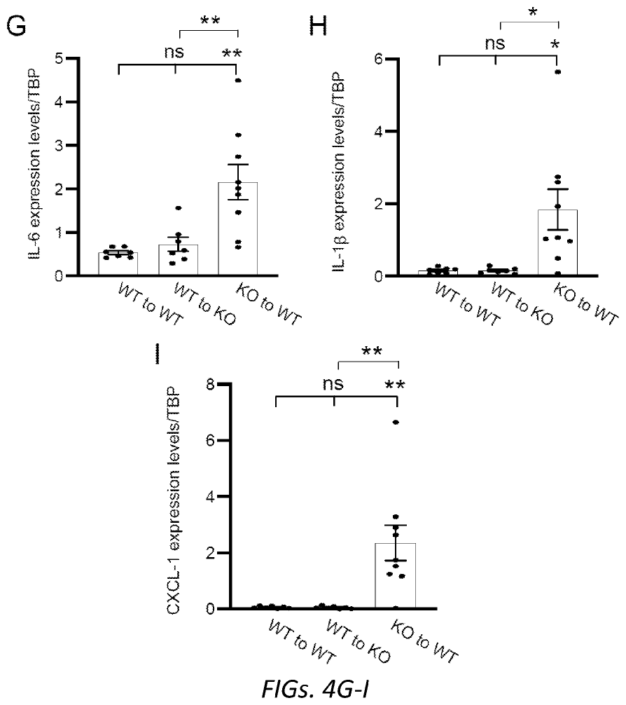

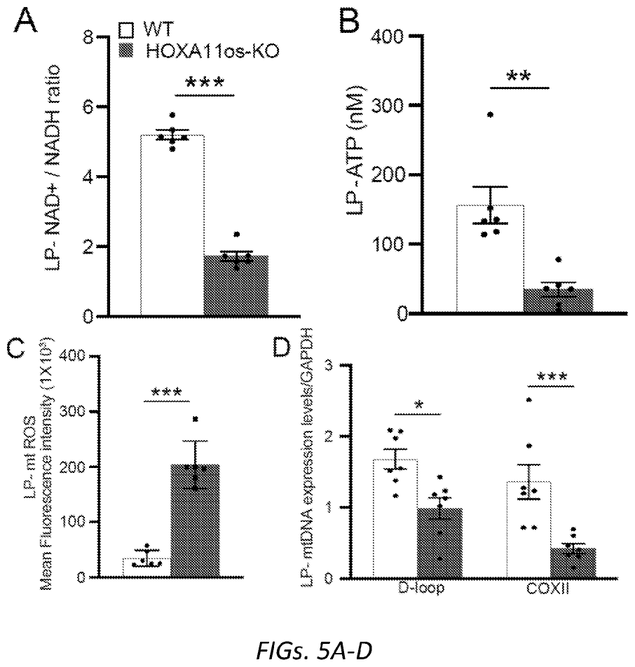

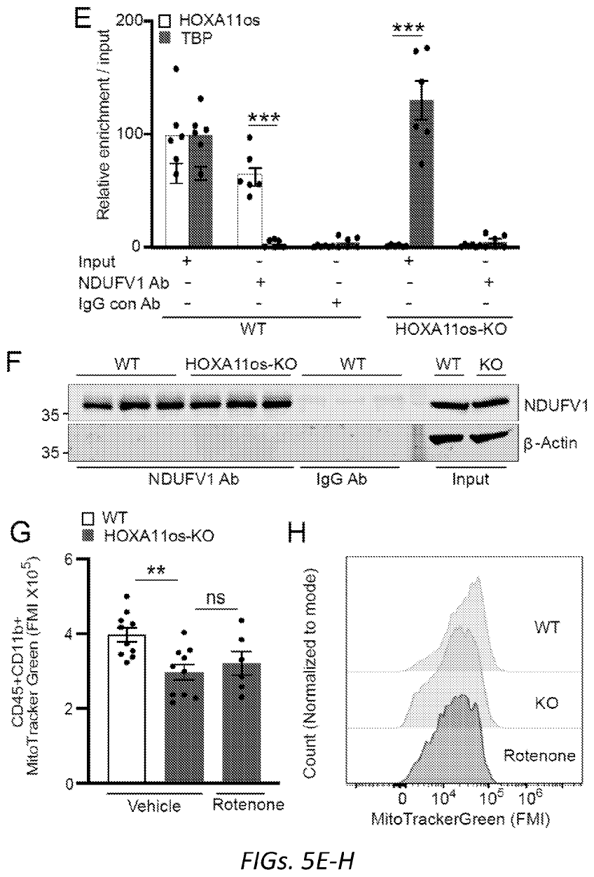

Given that HOXA11os is expressed in both IECs and immune cells of the distal colon (FIG. 7F-I), we next sought to understand the relative importance of each compartment in controlling intestinal homeostasis using bone marrow chimeras. Lethally Irradiated WT or HOXA11os-KO mice were reconstituted with bone marrow from either WT or HOXA11os-KO mice (FIG. 4A). Eight weeks post reconstitution, mice were treated with DSS for 5 days followed by a 7-day recovery period on regular water. As expected, WT mice reconstituted with WT bone marrow cells (WT to WT) recovered from DSS treatment (FIG. 4B-I). Similarly, HOXA11os-KO mice reconstituted with WT bone marrow (WT to KO) completely recovered from DSS treatment (FIG. 4B-I). Yet, WT mice reconstituted with KO bone marrow were hyper susceptible to colitis (FIG. 4B-I). Thus, HOXA11os is important in hematopoietic cells to maintain gut homeostasis and restrict colitis.

### Example 7. HOXA11Os Deficient Myeloid Cells Exhibits Impaired Complex I Activity

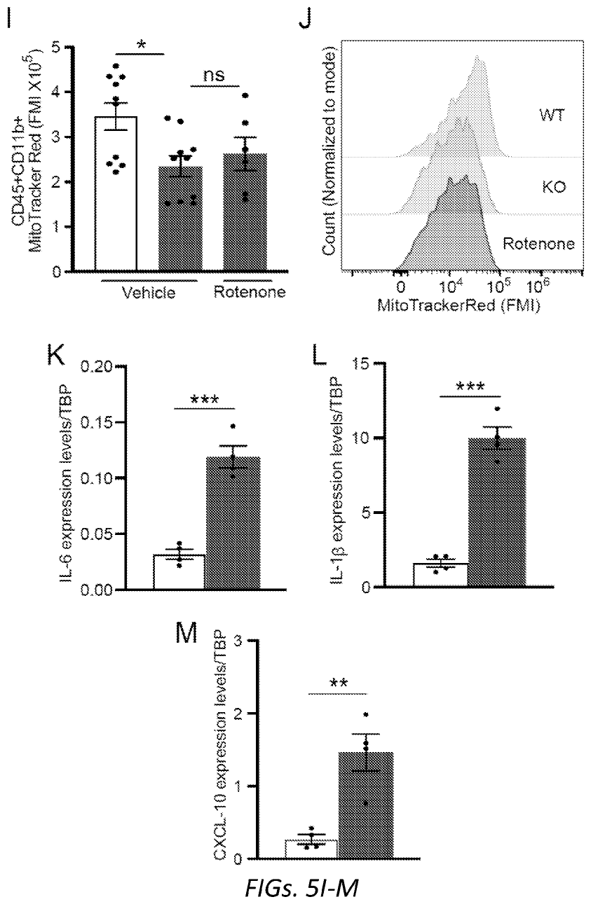

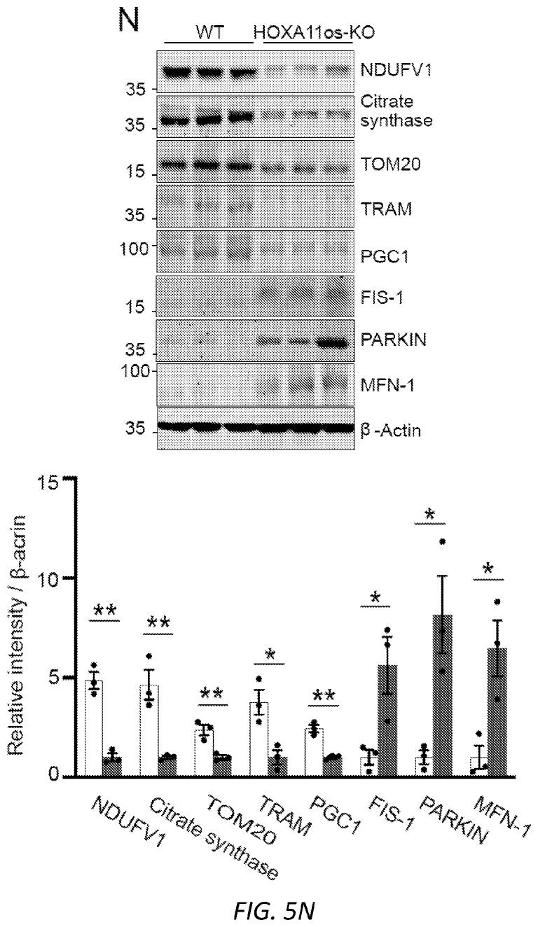

Fatty acids produced by the microbiota undergo fatty-acid oxidation to produce acetyl-CoA in colonocytes ^{42, 43}. Acetyl-CoA is then oxidized by the Krebs cycle coupled to the electron transport chain to produce ATP as part of OXPHOS. In this process, electrons from NADH and FADH2, produced as part of the Krebs cycle, are transferred down the mitochondrial respiratory complexes to molecular oxygen. In this process, protons are pumped from the mitochondrial matrix to the intermembrane space and oxygen is reduced to form water. The energy released from these oxidation/reduction reactions is used to drive the synthesis of ATP from ADP. Defects in beta oxidation ⁴⁴⁻⁴⁶, ETC and OXPHOS ³⁹⁻⁴¹ have been associated with intestinal inflammation. Given that HOXA11os deficiency in hematopoietic cells contributed to the increased susceptibility to colitis (FIG. 4), we next assessed mitochondrial metabolic activity of LP CD45⁺ immune cells (FIG. 5A-C). We observed reduced NAD+/NADH ratio (FIG. 5A), suggesting impaired activity of the ETC complex I. Impaired conversion of NADH to NAD was coupled with a reduction in ATP production (FIG. 5B). Inhibition of complex I leads to an increase in the production of mtROS due to the rapid generation of NADH 47, 48 Indeed, we observed elevated levels of mtROS in HOXA11os deficient immune cells (FIG. 5C). HOXA11os deficient immune cells also showed reduced levels of mitochondrial DNA (mtDNA) when compared to WT littermate controls (FIG. 5D). Reductions in the NAD+/NADH ratio, ATP production and elevated levels of mtROS were also observed in IECs from HOXA11os-KO mice. However, no alteration in mtDNA levels were found. Collectively, these data demonstrate that HOXA11os regulates complex I activity in colonic myeloid cells and protects against mitochondrial damage.

Next, we performed RIP (RNA Immunoprecipitation) coupled to qPCR 49 to confirm that HOXA11os interacts with the complex I subunit, NDUFV1 which we had identified via ChiRP-MS (FIG. 5E-F). HOXA11os was highly enriched in the RNA fraction that immunoprecipitated with NDUFV1 (FIG. 5E). HOXA11os interaction with NDUFV1 was specific, as we did not detect TBP in the NDUFV1 pulldowns or HOXA11os in our IgG isotope controls (FIG. 5E). In addition, HOXA11os was only detected in the input of WT cells but not of HOXA11os-KO cells, further demonstrating the specificity of the assay (FIG. 5E). The specificity of the pull down was confirmed by western blotting (FIG. 5F).

We next performed flow cytometry analysis of CD45⁺ CD11b⁺ myeloid cells, CD45⁺ CD11b⁻ lymphocytes and CD45 EPCM⁺ IECs to measure mitochondrial mass and mitochondrial membrane potential (ΔΨₘ) by using MitoTracker Green and MitoTracker Red, respectively ⁵⁰. We observed a significant decrease in both mitochondrial mass (FIG. 5G-H) and ΔΨₘ (FIG. 5I-J) in HOXA11os-KO myeloid cells. There was no further reduction of mitochondrial mass and ΔΨₘ in HOXA110s-KO myeloid cells treated with the complex I inhibitor, rotenone, indicating that complex 1 activity was already impaired in cells lacking HOXA11os ⁴⁷ (FIG. 5G-I). Comparable levels of mitochondrial mass and ΔΨₘ were observed in lymphocytes and IECs from HOXA11os-KO and WT littermate control mice. These data support a role for HOXA11os as a regulator of complex I activity and mitochondrial function in myeloid cells.

To provide additional evidence for the role of complex I deficiency in myeloid cells as the cause of inflammation in the colon of HOXA11os deficient mice, we extracted CD11b+ myeloid cells from older mice and assessed the levels of pro-inflammatory cytokines. As expected, we observed higher levels of pro-inflammatory cytokines in in HOXA11os-KO mice in comparison to their WT littermate counterparts (FIG. 5K-M).

We then assessed the levels of mitochondrial markers and regulators in the HOXA11os-KO immune cells (FIG. 5N) and IECs by western blotting. We observed reduced levels of NDUFV1 in HOXA11os-KO immune cells (FIG. 5N) but not IECs. HOXA11os-KO immune cells (FIG. 5N) but not IECs also demonstrated reduced levels of the mitochondrial mass markers, citrate synthase and the mitochondrial import receptor subunit TOM20 as well as a reduction in the protein levels of the mitochondrial biogenesis markers, translocating-chain-associated protein (TRAM) and PPARG coactivator 1 alpha (PGC1α). Mitochondrial fusion and fission play critical roles in maintaining functional mitochondria in response to environmental stress. Mitochondrial fusion serves to combine the contents of damaged mitochondria to healthy mitochondria whereas fission removes damaged mitochondria and facilitates mitophagy ⁵¹. Indeed, levels of the fusion protein mitofusion 1 (MFN-1), the mitochondrial fission protein 1 (Fis-1) and the mitophagy regulator PARKIN were all elevated in HOXA11os-KO immune cells (FIG. 5N) but not IECs.

We then assessed mitochondrial morphology by transmission electron microscopy (TEM). We observed that mitochondria from HOXA11os-KO myeloid cells were swollen with disrupted cristae and loss of matrix density when compared to intact mitochondrial ultrastructure in WT cells (FIG. 5O). Similar morphological changes have been previously reported by others in cells treated with rotenone ⁵² or cells with impaired ΔΨₘ ⁵³. Interestingly, mitochondrial morphology in IECs from HOXA11os-KO and WT littermate control mice demonstrated comparable, intact mitochondria. Taken together, these data demonstrate that HOXA11os deficiency in colonic myeloid cells compromises mitochondrial function via dysregulated complex I.

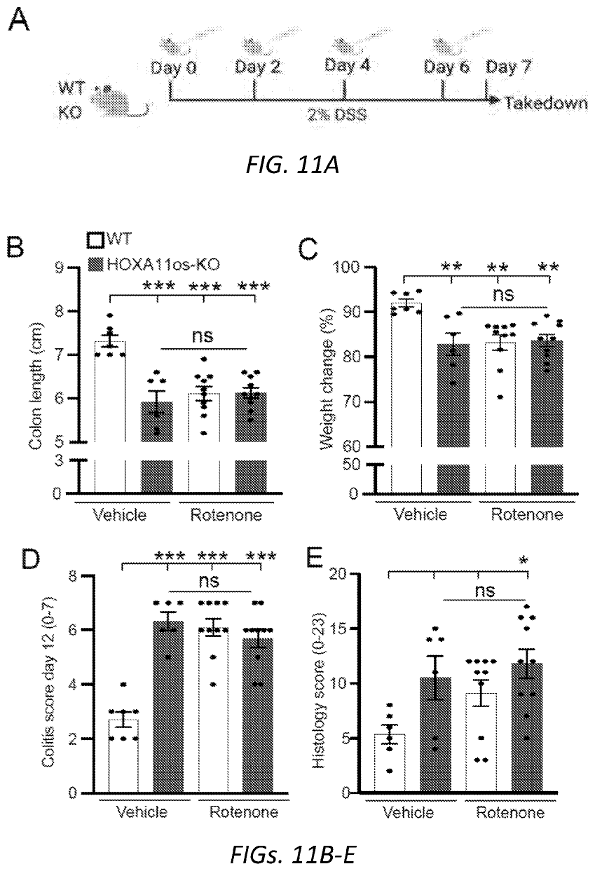

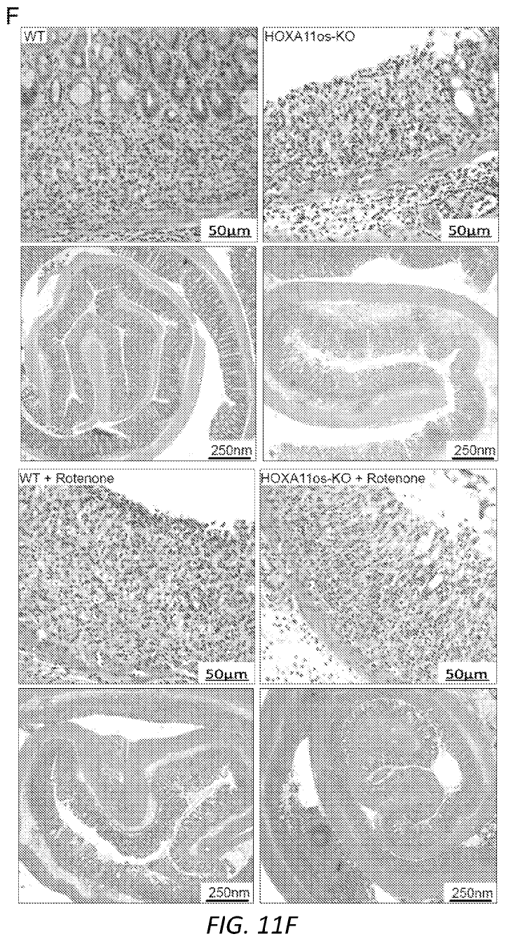

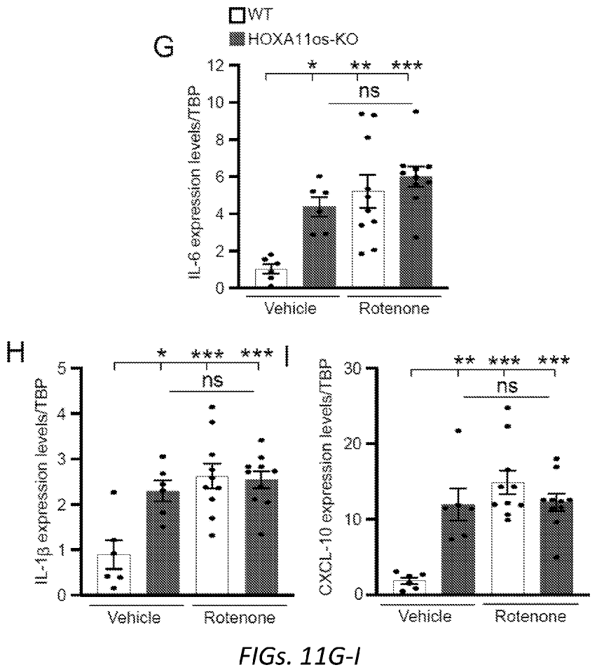

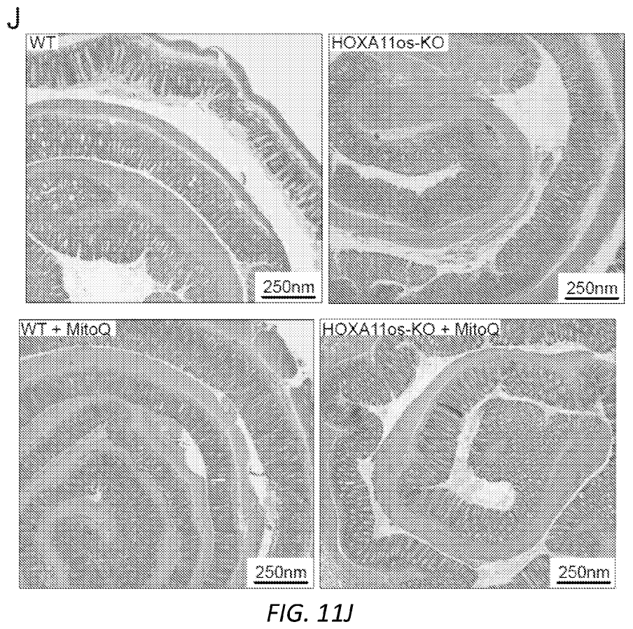

To confirm that deficiency of complex I is responsible for the exacerbated colitis in HOXA11os-KO mice, we treated mice with the complex I inhibitor rotenone. Rotenone was administrated locally via rectal administration to HOXA11os-KO and WT littermate control mice every other day in the presence of 2% DSS (FIG. 11A). WT mice treated with rotenone were more susceptible to colitis to a degree similar to that observed in HOXA11os-KO mice, as demonstrated by shorter colons (FIG. 11B), greater weight loss (FIG. 11B), traces of blood and diarrhea in the feces (FIG. 11D), greater pathological score (FIG. 11E-F) and elevated levels of pro-inflammatory cytokines (FIG. 11G-I). Rotenone treatment of HOXA11os-KO mice had no further impact on these mice. These data demonstrate that dysfunctional mitochondria as a result of complex I inhibition by rotenone or due to HOXA11os-deficiency leads to enhanced susceptibility to colitis.

### Example 8. Treatment with the mtROS Scavenger, MitoQ, Ameliorated Colitis in HOXA11Os-KO Mice

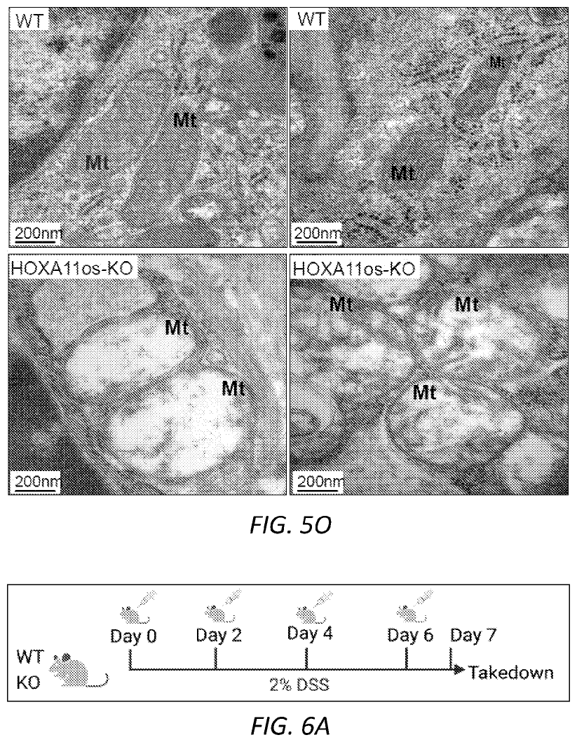

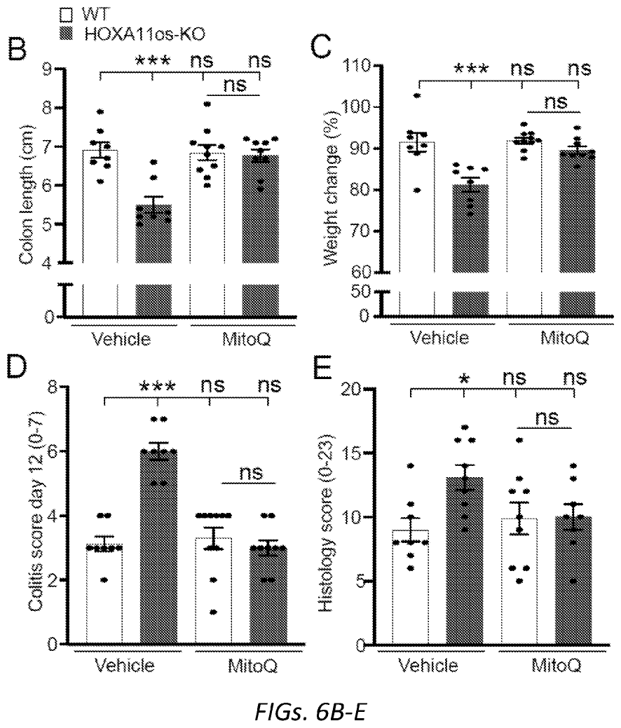

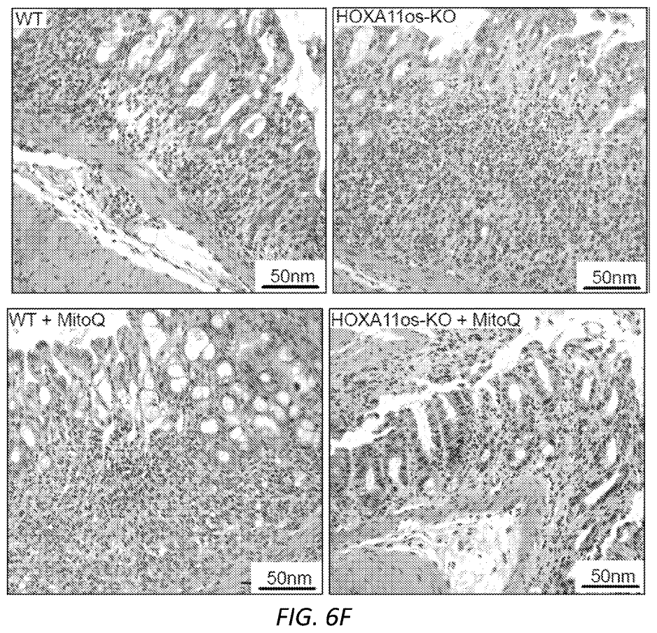

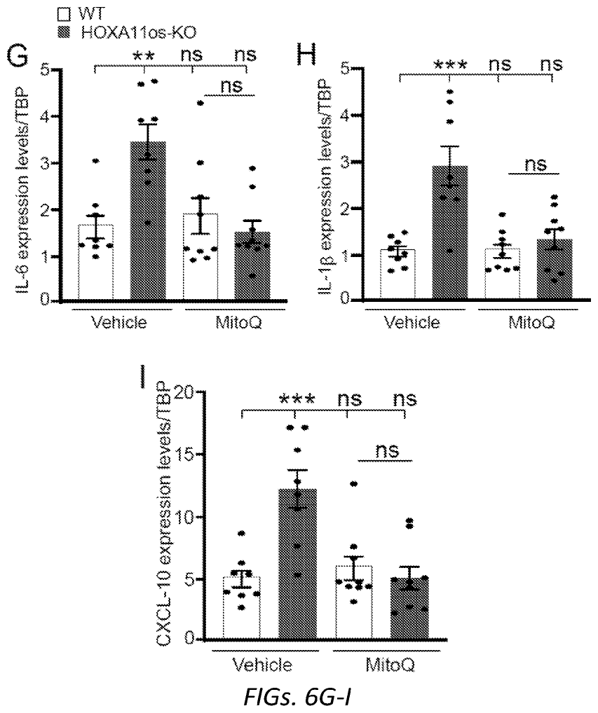

Complex I deficiency leads to elevated mtROS in HOXA11os-KO immune cells (FIG. 5C). Excessive mtROS can cause oxidative stress, induction of inflammatory responses and activation of cell death pathways. MitoQ is a mitochondrial anti-oxidant that accumulates within the mitochondrial matrix to scavenge mtROS ⁵⁴. To test if mtROS contributed to the exacerbated colitis phenotype observed in HOXA11os-KO mice, HOXA11os-KO and WT littermate control mice were treated with MitoQ every other day in the presence of 2% DSS for 7 days (FIG. 6A). MitoQ ameliorated the enhanced colitis in HOXA11os-KO mice treated with DSS. Indeed, MitoQ treated HOXA11os-KO mice displayed comparable colon length (FIG. 6B), weight loss (FIG. 6C), blood and diarrhea (FIG. 6D), pathological scores (FIG. 6E-F, FIG. 11J) and pro-inflammatory cytokines (FIG. 6G-I) as WT littermate control mice.

Collectively, these studies identify and characterize a lncRNA, HOXA11os, that under basal conditions maintains complex I activity in colonic myeloid cells. In the absence of HOXA11os, colonic myeloid cells have defective mitochondria, resulting in excessive production of mtROS and hyper susceptibility to colitis.

TABLE 1
Murine HOXA11os transcripts sequences.

| | | SEQ |
|---|---|---|
| | | ID |
| Transcripts | Sequence | NO: |
| HOXA11os-1 | CTTCCTTTCTTTGTAGCCACCTCAGGGGAAGCAACAGATCGTCACTCGGTGTTCTCACCGAAA | 1 |
| | GCACGTAATCGCCGGTGTAACTCATGTTGGCTGGGGGGCCTCCCCGCGCGCAGAAAGGCTGGG | |
| | GTGCGCCCCCGGGCAGCTCTCCTTTGCTCAGCTACATGGTCCTGGTCCACGAGTGCTCTGAGG | |
| | GCGGCAAGAGAGCGCAACTCCTGACGCCTCCCCCCACTCCCCGGGGTTCGGAGTGACTCCTCA | |
| | GAGCCAGAGGCACTTCTGCTCACCGGTCCGCAAGCTGCCTGGTCTGCTGAAGCTGACGAATCG | |
| | GGAAACCATGCAATTGAGGCGAACCTTGGGCTGTTTTAGAGGCGCTGAGGAGCCTTCTCCTGG | |
| | GAGGCCCAAGGTTGATTTCAGCCCACCAGGATCTGGGGAAGACCCAACTAGGGATAAGAGCAC | |
| | ACCAAAAGGCCAAGTCCGAGTTCCATTTCTAGAAGAGGCGGCTTCCGGCAAGGCTATGACATT | |
| | GGCCCTGGACATTGGTTTCCCAGGAGCTGCTTTTTCTCAAGAACTCCACAGCACGGGGCTGTC | |
| | TCCAGAAAACGCTCTTCAACGTTTATTTCTTTTAATCGTCGCCCGGAGCCCTAAGGCGGCTAA | |
| | TGCAAGAGGCCAAAAATGTTTGGAGGAAGAAAAACAAAGGCAGGAAGTGGCCGCGGCCTGACG | |
| | GTGCGTGTGTGTCTGTAAAGAAGGGAGGGAGCCGGTTCAACCTCCCCTCGTTTTCCCGAACTT | |
| | CAAGGTCTAGGCAGACCCCCTTAGGGCCTTGCCGAGGCTCGCCCCCACACCCCCAGCGGCGCA | |
| | GCATTTGGAGGTGGCCAACGATTTAAGCCTCGGTCGGGCTGAAAGGAGATTTGATCGGCAGAA | |
| | CAAACCAACCCTTTTCGGAGGTTTCTTTTGATTTGGTCCTAAAGGGTATATGCTAGTGTCCAC | |
| | AGCGGCTGGGGTGGCTGCTGTTTTCCTCCCGCCGGGCTAAAAGTACCAAGAAGGGAGGGAGGG | |
| | AGAGAGATTCAGGCACCTTGCGCTGGCTGCACTCTCCTTCTGAGATAGAATACCAGAATAAAG | |
| | TGTATTCAGGTGCC | |
| HOXA11os-2 | CTTCCTTTCTTTGTAGCCACCTCAGGGGAAGCAACAGATCGTCACTCGGTGTTCTCACCGAAA | 2 |
| | GCACGTAATCGCCGGTGTAACTCATGTTGGCTGGGGGGCCTCCCCGCGCGCAGAAAGGCTGGG | |
| | GTGCGCCCCCGGGCAGCTCTCCTTTGCTCAGCTACATGGTCCTGGTCCACGAGTGCTCTGAGG | |
| | GCGGCAAGAGAGCGCAACTCCTGACGCCTCCCCCCACTCCCCGGTGGGTGAGGGATACTCTCT | |
| | GGGCTGGGGGTGGCCAGGTGAAAGCCCGGAATTGTATAGCTTCAGGTCCCGGAGTCTGTTATC | |
| | CGAAGGCTTACGTTCAGCACCTTCTTCGCAGCCCCCCTCCCACAGGTTTGCTCTGGGAAGCCC | |
| | CCCAGCCTCAGACCCTGGCTGGACCCCATTTGGGGCCAGGCTTCGCCGGCACGGATGTGCCGG | |
| | CCTCGTGGCTTGTCCGATTTGCACGGTGACTTGATTACACGCTCTCATTCACGGTCACTTCCG | |
| | AAGCGCTTTAGTGCCTTCCGTCCCCAAACCGCCAACAGGCAAAGCGGCTTCCCTCCGCGGTTT | |
| | GTCAATAATCCGCGCTGTCCGGAAGGGCCTTCGTCTTACCCGGGTTCCACCTTCCCTGTATCT | |
| | TTCTGCTTACTTCCTTACCCCACACCCTGTCCTTGAAGGAACCCCTTCTCCTCGCTGCCTGTA | |
| | GGGGTTCGGAGTGACTCCTCAGAGCCAGAGGCACTTCTGCTCACCGGTCCGCAAGCTGCCTGG | |
| | TCTGCTGAAGCTGACGAATCGGGAAACCATGCAATTGAGGCGAACCTTGGGCTGTTTTAGAGG | |
| | CGCTGAGGAGCCTTCTCCTGGGAGGCCCAAGGTTGATTTCAGCCCACCAGGATCTGGGGAAGA | |
| | CCCAACTAGGGATAAGAGCACACCAAAAGGCCAAGTCCGAGTTCCATTTCTAGAAGAGGCGGC | |
| | TTCCGGCAAGGCTATGACATTGGCCCTGGACATTGGTTTCCCAGGAGCTGCTTTTTCTCAAGA | |
| | ACTCCACAGCACGGGGCTGTCTCCAGAAAACGCTCTTCAACGTTTATTTCTTTTAATCGTCGC | |
| | CCGGAGCCCTAAGGCGGCTAATGCAAGAGGCCAAAAATGTTTGGAGGAAGAAAAACAAAGGCA | |
| | GGAAGTGGCCGCGGCCTGACGGTGCGTGTGTGTCTGTAAAGAAGGGAGGGAGCCGGTTCAACC | |
| | TCCCCTCGTTTTCCCGAACTTCAAGGTCTAGGCAGACCCCCTTAGGGCCTTGCCGAGGCTCGC | |
| | CCCCACACCCCCAGCGGCGCAGCATTTGGAGGTGGCCAACGATTTAAGCCTCGGTCGGGCTGA | |
| | AAGGAGATTTGATCGGCAGAACAAACCAACCCTTTTCGGAGGTTTCTTTTGATTTGGTCCTAA | |
| | AGGGTATATGCTAGTGTCCACAGCGGCTGGGGTGGCTGCTGTTTTCCTCCCGCCGGGCTAAAA | |
| | GTACCAAGAAGGGAGGGAGGGAGAGAGATTCAGGCACCTTGCGCTGGCTGCACTCTCCTTCTG | |
| | AGATAGAATACCAGAATAAAGTGTATTCAGGTGCCTCTGC | |
| HOXA11os-3 | ATTTAAAAACAATTGTGAATAACAGAAGGCAGCATGTAATTGCAACCGGACCAGAGGAAAATG | 3 |
| | GCTTCCTTTGGACAAAAAGGCCAAATTTAAGTTAATTTGTTTCTTTTAATATATTGCTTGAGC | |
| | TAAGTGGGCTACTTTATCTGTTAAATGCGCTCTTAGCGCCATCATTAAACAGCCGATGCCTTT | |
| | GAATCCCAGCCGGGACTGGAGTCCCGCCGCAACACATGGCTTTTATAAAAATCTCCGGATTAC | |
| | CCCGCTATCAAAGGCTCCTGAAGCCCTTGTAGGGGGAATTTACAGGCGCCCACCTCCGGCTCC | |
| | CCAGCCTGGAGCTGGTCTCTAGCGGGGATCGTGCTGTTGAGTTTGGGAGCTGAGAAGGGAAAA | |
| | AAGGAAAAAGGGGAGTTGTATTTTTTTTTTTTTCTGCAGGCATGCTTTGGCCGGTGGGTATTT | |
| | CACGGCCAATTTCAGCACTCGCCACGTGATCCCTCCTTTTATAACAAAGTTTTGTTGGGGGAA | |
| | ACCTAAAGGCCCTTCATAAACCTTATATGCTTATAAAACAGCATATAAAAATTTAACAGCGGT | |
| | GCTGCGCTAGATTCCCAACTCCCCTTTCATAAAGCGCTGGGCGCTGCCTTTATACGCACCGGA | |
| | GCCACCGGCCTTGTCCTCAGTGTGGCCGGAAGACGACTCGGGGCTGCTGCTGCTCTCGGGTCG | |
| | CCGCCGTCGCTCCTTCTCCTCCGCCGCCGCCTCCTGACAGCCGCCGCCACCGCCGCCGTCCGA | |
| | ACTTGAAGTTGCCGGCGCGCCCGTGGCCGCCGCCGCCACCGCCGCAGCGGAGGTCGCAGCTGC | |
| | TGCTTGGGGCCCCTTCTCGGCGTTCTTGTCCCCGGGGTAGTCGGAGGAAGCGAGGTTTTCCGG | |
| | GGTGCCGTAAGCCGTCTCGAAAAACTGGTCGAAAGCCTGTGGCAGAACGCCGTTCCTGCCCAC | |
| | CGTGCTATAGAAATTGGACGAGACGGCGGGGGTGGGGTGGTGGTAGACGTTGGCCGAGCTCTT | |
| | GGCCAGCACGTCGCCAGGCACGCCGGCCGCGCTGGGCGCCTGCAGACAGTCTCTGTGCACGAG | |
| | CTCCTCCGCGGAGTAGCAGTGGGCCAGATTGCCGCGGGGGTGCCATTTAGTGGCGGGCTCAAT | |
| | GGCGTACTCTCTGAAGGTCACTTCGCGCACGGGTTGGACCTGGGGCAGGTTGGAGGAGTAGGA | |
| | GTATGTCATTGGGCGCGAAGACGGGGTCTGGGGCAAAAAAGAAGGGAGGCTGGAGAAATCTGG | |
| | ACCCGAGACGTAGGGTTCGGAGTGACTCCTCAGAGCCAGAGGCACTTCTGCTCACCGGTCCGC | |
| | AAGCTGCCTGGTCTGCTGAAGCTGACGAATCGGGAAACCATGCAATTGAGGCGAACCTTGGGC | |
| | TGTTTTAGAGGCGCTGAGGAGCCTTCTCCTGGGAGGCCCAAGGTTGATTTCAGCCCACCAGGA | |
| | TCTGGGGAAGACCCAACTAGGGATAAGAGCACACCAAAAGGCCAAGTCCGAGTTCCATTTCTA | |
| | GAAGAGGCGGCTTCCGGCAAGGCTATGACATTGGCCCTGGACATTGGTTTCCCAGGAGCTGCT | |
| | TTTTCTCAAGAACTCCACAGCACGGGGCTGTCTCCAGAAAACGCTCTTCAACGTTTATTTCTT | |
| | TTAATCGTCGCCCGGAGCCCTAAGGCGGCTAATGCAAGAGGCCAAAAATGTTTGGAGGAAGAA | |
| | AAACAAAGGCAGGAAGTGGCCGCGGCCTGACGGTGCGTGTGTGTCTGTAAAGAAGGGAGGGAG | |
| | CCGGTTCAACCTCCCCTCGTTTTCCCGAACTTCAAGGTCTAGGCAGACCCCCTTAGGGCCTTG | |
| | CCGAGGCTCGCCCCCACACCCCCAGCGGCGCAGCATTTGGAGGTGGCCAACGATTTAAGCCTC | |
| | GGTCGGGCTGAAAGGAGATTTGATCGGCAGAACAAACCAACCCTTTTCGGAGGTTTCTTTTGA | |
| | TTTGGTCCTAAAGGGTATATGCTAGTGTCCACAGCGGCTGGGGTGGCTGCTGTTTTCCTCCCG | |
| | CCGGGCTAAAAGTACCAAGAAGGGAGGGAGGGAGAGAGATTCAGGCACCTTGCGCTGGCTGCA | |
| | CTCTCCTTCTGAGATAGAATACCAGAATAAAGTGTATTCAGGTGCCT | |
| HOXA11os-4 | CTTCCTTTCTTTGTAGCCACCTCAGGGGAAGCAACAGATCGTCACTCGGTGTTCTCACCGAAA | 4 |
| | GCACGTAATCGCCGGTGTAACTCATGTTGGCTGGGGGGCCTCCCCGCGCGCAGAAAGGCTGGG | |
| | GTGCGCCCCCGGGCAGCTCTCCTTTGCTCAGCTACATGGTCCTGGTCCACGAGTGCTCTGAGG | |
| | GCGGCAAGAGAGCGCAACTCCTGACGCCTCCCCCCACTCCCCGGAACCCCTTCTCCTCGCTGC | |
| | CTGTAGGGGTTCGGAGTGACTCCTCAGAGCCAGAGGCACTTCTGCTCACCGGTCCGCAAGCTG | |
| | CCTGGTCTGCTGAAGCTGACGAATCGGGAAACCATGCAATTGAGGCGAACCTTGGGCTGTTTT | |
| | AGAGGCGCTGAGGAGCCTTCTCCTGGGAGGCCCAAGGTTGATTTCAGCCCACCAGGATCTGGG | |
| | GAAGACCCAACTAGGGATAAGAGCACACCAAAAGGCCAAGTCCGAGTTCCATTTCTAGAAGAG | |
| | GCGGCTTCCGGCAAGGCTATGACATTGGCCCTGGACATTGGTTTCCCAGGAGCTGCTTTTTCT | |
| | CAAGAACTCCACAGCACGGGGCTGTCTCCAGAAAACGCTCTTCAACGTTTATTTCTTTTAATC | |
| | GTCGCCCGGAGCCCTAAGGCGGCTAATGCAAGAGGCCAAAAATGTTTGGAGGAAGAAAAACAA | |
| | AGGCAGGAAGTGGCCGCGGCCTGACGGTGCGTGTGTGTCTGTAAAGAAGGGAGGGAGCCGGTT | |
| | CAACCTCCCCTCGTTTTCCCGAACTTCAAGGTCTAGGCAGACCCCCTTAGGGCCTTGCCGAGG | |
| | CTCGCCCCCACACCCCCAGCGGCGCAGCATTTGGAGGTGGCCAACGATTTAAGCCTCGGTCGG | |
| | GCTGAAAGGAGATTTGATCGGCAGAACAAACCAACCCTTTTCGGAGGTTTCTTTTGATTTGGT | |
| | CCTAAAGGGTATATGCTAGTGTCCACAGCGGCTGGGGTGGCTGCTGTTTTCCTCCCGCCGGGC | |
| | TAAAAGTACCAAGAAGGGAGGGAGGGAGAGAGATTCAGGCACCTTGCGCTGGCTGCACTCTCC | |
| | TTCTGAGATAGAATACCAGAATAAAGTGTATTCAGGTGCCTCTGC | |

TABLE 2
Human HOXA11AS transcripts sequences.

| | | SEQ |
|---|---|---|
| | | ID |
| Transcripts | Sequence | NO: |
| HOXA11AS-1 | GCCACCTCAGGGGAAGCAACAGATCGTCACTCGGTGTTCTCACCGAAAGCACGTAATCGCCGG | 5 |
| | TGTAACTCATGTTGGCTGGGGGGCCTCCCGGCGCGCGCGGAGAGGCTGGGGTGCGCCCCCATG | |
| | CAGCATGCTTGTGCTCAATTGCAGGGTCCTCGTTCTCGAGTGTGCAGAGGGCGGTGAGAGCTC | |
| | AACTCTCGTCCCCACCTCCCACCCGCAGCTCCCCGGGTGGGTGAGGGATGCCCTGGACTGGGG | |
| | ATAGCCAGGTGGGAGTCCGTCGCTGTGTGGCCTGTGGTCTCGGAGTCTGTTCTCCTGGAGTCT | |
| | CGCATTTGCACCCCCTTCTTCGCAGTCCCCCTCCCATAGACTTGCTCTGGGAAGCGCCTCTGC | |
| | CTCCGACCCTAGCCGGAACCCCTTCGGGGCCAGAGTTTGAAGCCGTGGATGTGCCTGCCTGGT | |
| | GGCTTGTCCGATTTGCACGGTGACTTGATTACACTCTCTCATTCATGGTCACTTCCGAAGCGC | |
| | TTTAGTGCCTTCCGTCCCTAAACCGCCAACAGCCAGAACGGCTTCTCCCCGCGGTTTGTCACT | |
| | GATCCGCAGGGCCCGGAAGGGCCTTCGTCTTACCCGGGATCCACCTCTCCCCTCATCTTCCCT | |
| | GCCTACCTCTTCATCCCACCTTCTGTCCTTGGAGAAACTCCCTCCTCCTCGCTGCCTGCCGGG | |
| | CTTCGGAGTGACTCGGCAGAGACAGAGGCACAGGGGCTGCCCTGCTGCTCACCGGTCCACCCA | |
| | TCTGCCTGGTCTTCTGGAGCTGAGGACTCGGGAAACCATGCAATTGAGGCAAGCCTTGGGCTG | |
| | CTTTAGAGGCGCTGACATCCGAGGAGACTTCTCCTGGGAGGTCCAACAGCCGAGCTTAGCCCA | |
| | CCGGGCTCTGGGAAAGACCCGACTGAGGCTAAAGCCGCCCCGGAAGGCCAAGTCCGAGTTCCA | |
| | TTTCTTGAAGAGGCCGGCGCGCGTAAGGCTGTGACATTGGCCCTGGCGACTGGCTTCCCAGGA | |
| | GCTGTTCTTTCTCAGGAGCTCCACAGCGCGGGCCATCTCCAGAAAACTGTCTTCAGAGTGTAT | |
| | TTCCTTTTATCGTCAACCCAGAGCCCCACCGCGGCTAATGCAAGAGGCCAAAAAATGTTTGGA | |
| | GGAAGAAAAACAAAGGCAGGAAGTGGCGGCGGCCTGACGGTGCGTGTGTGTCTGCAGAGAAGG | |
| | GAGGGAGCCGGCTCAGTCTCTTCTTGTTTTTCCAAACTTCAAGGTCCAGGCAGCCCTCTGCAG | |
| | GGCCGGGCCCCATTGCTCCCCGCGCGGCATTGGAGGTGGCCGCCCGGAGAGGAGAAGGCCAAC | |
| | GCCTGCGCCAGGCTTGTCAGGCGGAAACGGCTAACAAGGAGATTTGGTCAGCAAAACAGACCC | |
| | AGCCTTTCCGAGGCTTCGTCTGACTTGGCCCGAAAGGTTGGGGAGGGGGGGCTTGCGCAGAGC | |
| | CTCAGGGACCCTCCTCTCTGGGGACTACCATCCCTGAGCCTTACGCTTCTTTCCACAGCCTTT | |
| | GCAGGCGGAATATCGGAATAAAGTGGGICCAGGCGCC | |
| HOXA11AS-2 | CAGCATGCTTGTGCTCAATTGCAGGGTCCTCGTTCTCGAGTGTGCAGAGGGCGGTGAGAGCTC | 6 |
| | AACTCTCGTCCCCACCTCCCACCCGCAGCTCCCCGGGTGGGTGAGGGATGCCCTGGACTGGGG | |
| | ATAGCCAGGTGGGAGTCCGTCGCTGTGTGGCCTGTGGTCTCGGAGTCTGTTCTCCTGGAGTCT | |
| | CGCATTTGCACCCCCTTCTTCGCAGTCCCCCTCCCATAGACTTGCTCTGGGAAGCGCCTCTGC | |
| | CTCCGACCCTAGCCGGAACCCCTTCGGGGCCAGAGTTTGAAGCCGTGGATGTGCCTGCCTGGT | |
| | GGCTTGTCCGATTTGCACGGTGACTTGATTACACTCTCTCATTCATGGTCACTTCCGAAGCGC | |
| | TTTAGTGCCTTCCGTCCCTAAACCGCCAACAGCCAGAACGGCTTCTCCCCGCGGTTTGTCACT | |
| | GATCCGCAGGGCCCGGAAGGGCCTTCGTCTTACCCGGGATCCACCTCTCCCCTCATCTTCCCT | |
| | GCCTACCTCTTCATCCCACCTTCTGTCCTTGGAGAAACTCCCTCCTCCTCGCTGCCTGCCGGG | |
| | CTTCGGAGTGACTCGGCAGAGACAGAGGCACAGGGGCTGCCCTGCTGCTCACCGGTCCACCCA | |
| | TCTGCCTGGTCTTCTGGAGCTGAGGACTCGGGAAACCATGCAATTGAGGCAAGCCTTGGGCTG | |
| | CTTTAGAGGCGCTGACATCCGAGGAGACTTCTCCTGGGATTGAAGGAGCAATGTTTGGAGGAA | |
| | GCGAAAGAAAGGAGAGGACACAGAGCACAGAGCAGCCAGGCAGAGCCAGGAGCTGAGAAGGGC | |
| | CCAGACCTGAGGCCTCCCAACAACTCTCTTCTTGGAAGGATCTGGGATGTTGCTGAAGGAAAA | |
| | TAAAAAAATATGTAAAAAGATAACCTTTTGTTTTTCCCTCTCCAGGAAATAGCCAAAGTTATT | |
| | TACATATCTTGGGGAGATTTAGAGTATAAACTCTAAGATCTTTGGTATTTAAGTGTCAACATC | |
| | GATTTATTTATTTATTGCTGAGCTGACTGTAACTGACTCAATAACAAATCTAATCGTGTATTG | |
| | CACTGGAAAAGAAATATTCTTATTATGTATTTTCTCCAAATAATGGCCTACCATTGCATTTGA | |
| | ATACCTGCTGTAAATATCAATAATATGAAGTAATTACTCTGTAGTCGAGTAAACTAATTTATT | |
| | AGCA | |

TABLE 3
Murine HOXA11os putative ORF sequences.

| | | SEQ ID |
|---|---|---|
| ORF | Sequence | NO: |
| 1 | ATGCAATTGAGGCGAACCTTGGGCTGTTTTAGAGGCGCTGAGGAGCCTTCTCCTGGGAGGCCCAAGGTTGATT | 7 |
| | TCAGCCCACCAGGATCTGGGGAAGACCCAACTAGGGATAAGAGCACACCAAAAGGCCAAGTCCGAGTTCCATT | |
| | TCTAGAAGAGGCGGCTTCCGGCAAGGCTATGACATTGGCCCTGGACATTGGTTTCCCAGGAGCTGCTTTTTCT | |
| | CAAGAACTCCACAGCACGGGGCTGTCTCCAGAAAACGCTCTTCAACGTTTATTTCTTTTAATCGTCGCCCGGA | |
| | GCCCTAAGGCGGCTAATGCAAGAGGCCAAAAATGTTTGGAGGAAGAAAAACAAAGGCAGGAAGTGGCCGCGGC | |
| | CTGA | |
| 2 | ATGCAAGAGGCCAAAAATGTTTGGAGGAAGAAAAACAAAGGCAGGAAGTGGCCGCGGCCTGACGGTGCGTGTG | 8 |
| | TGTCTGTAAAGAAGGGAGGGAGCCGGTTCAACCTCCCCTCGTTTTCCCGAACTTCAAGGTCTAGGCAGACCCC | |
| | CTTAGGGCCTTGCCGAGGCTCGCCCCCACACCCCCAGCGGCGCAGCATTTGGAGGTGGCCAACGATTTAA | |
| 3 | ATGTTGGCTGGGGGGCCTCCCCGCGCGCAGAAAGGCTGGGGTGCGCCCCCGGGCAGCTCTCCTTTGCTCAGCT | 9 |
| | ACATGGTCCTGGTCCACGAGTGCTCTGAGGGCGGCAAGAGAGCGCAACTCCTGACGCCTCCCCCCACTCCCCG | |
| | GGGTTCGGAGTGA | |
| 4 | ATGTTGGCTGGGGGGCCTCCCCGCGCGCAGAAAGGCTGGGGTGCGCCCCCGGGCAGCTCTCCTTTGCTCAGCT | 10 |
| | ACATGGTCCTGGTCCACGAGTGCTCTGAGGGCGGCAAGAGAGCGCAACTCCTGACGCCTCCCCCCACTCCCCG | |
| | GTGGGTGAGGGATACTCTCTGGGCTGGGGGTGGCCAGGTGAAAGCCCGGAATTGTATAGCTTCAGGTCCCGGA | |
| | GTCTGTTATCCGAAGGCTTACGTTCAGCACCTTCTTCGCAGCCCCCCTCCCACAGGTTTGCTCTGGGAAGCCC | |
| | CCCAGCCTCAGACCCTGGCTGGACCCCATTTGGGGCCAGGCTTCGCCGGCACGGATGTGCCGGCCTCGTGGCT | |
| | TGTCCGATTTGCACGGTGA | |
| 5 | ATGTGCCGGCCTCGTGGCTTGTCCGATTTGCACGGTGACTTGATTACACGCTCTCATTCACGGTCACTTCCGA | 11 |
| | AGCGCTTTAGTGCCTTCCGTCCCCAAACCGCCAACAGGCAAAGCGGCTTCCCTCCGCGGTTTGTCAATAATCC | |
| | GCGCTGTCCGGAAGGGCCTTCGTCTTACCCGGGTTCCACCTTCCCTGTATCTTTCTGCTTACTTCCTTACCCC | |
| | ACACCCTGTCCTTGA | |

### REFERENCES

1. Djebali S, Landscape of transcription in human cells. Nature. 2012; 489(7414):101-8. Epub 2012 Sep. 8. doi: 10.1038/nature11233. PubMed PMID: 22955620; PMCID: PMC3684276.
2. Guttman et al., Chromatin signature reveals over a thousand highly conserved large non-coding RNAs in mammals. Nature. 2009; 458(7235):223-7. Epub 2009 Feb. 3. doi: 10.1038/nature07672. PubMed PMID: 19182780; PMCID: PMC2754849
3. Dinger M E, Pang K C, Mercer T R, Crowe M L, Grimmond S M, Mattick J S. NRED: a database of long noncoding RNA expression. Nucleic Acids Res. 2009; 37 (Database issue): D122-6. Epub 2008 Oct. 3. doi: 10.1093/nar/gkn617. PubMed PMID: 18829717; PMCID: PMC2686506.
4. Carpenter S. Determining the Function of Long Noncoding RNA in Innate Immunity. Methods Mol Biol. 2016; 1390:183-95. Epub 2016 Jan. 25. doi: 10.1007/978-1-4939-3335-8_12. PubMed PMID: 26803630.
5. Carpenter S, Aiello D, Atianand M K, Ricci E P, Gandhi P, Hall L L, Byron M, Monks B, Henry-Bezy M, Lawrence J B, O'Neill L A, Moore M J, Caffrey D R, Fitzgerald K A. A long noncoding RNA mediates both activation and repression of immune response genes. Science. 2013; 341(6147):789-92. Epub 2013 Aug. 3. doi: 10.1126/science. 1240925. PubMed PMID: 23907535; PMCID: PMC4376668.
6. Kotzin J J, Spencer S P, McCright S J, Kumar D B U, Collet M A, Mowel W K, Elliott E N, Uyar A, Makiya M A, Dunagin M C, Harman C C D, Virtue A T, Zhu S, Bailis W, Stein J, Hughes C, Raj A, Wherry E J, Goff L A, Klion A D, Rinn J L, Williams A, Flavell R A, Henao-Mejia J. The long non-coding RNA Morrbid regulates Bim and short-lived myeloid cell lifespan. Nature. 2016; 537(7619):239-43. Epub 2016 Aug. 16. doi: 10.1038/nature19346. PubMed PMID: 27525555; PMCID: PMC5161578.
7. Atianand M K, Hu W, Satpathy A T, Shen Y, Ricci E P, Alvarez-Dominguez J R, Bhatta A, Schattgen S A, McGowan J D, Blin J, Braun J E, Gandhi P, Moore M J, Chang H Y, Lodish H F, Caffrey D R, Fitzgerald K A. A Long Noncoding RNA lincRNA-EPS Acts as a Transcriptional Brake to Restrain Inflammation. Cell. 2016; 165(7):1672-85. Epub 2016 Jun. 18. doi: 10.1016/j.cell.2016.05.075. PubMed PMID: 27315481; PMCID: PMC5289747.
8. Atianand M K, Fitzgerald K A. Long non-coding RNAs and control of gene expression in the immune system. Trends Mol Med. 2014; 20(11):623-31. Epub 2014 Sep. 30. doi: 10.1016/j.molmed.2014.09.002. PubMed PMID: 25262537; PMCID: PMC4252818.
9. Li Z, Chao T C, Chang K Y, Lin N, Patil V S, Shimizu C, Head S R, Burns J C, Rana™. The long noncoding RNA THRIL regulates TNFalpha expression through its interaction with hnRNPL. Proc Natl Acad Sci USA. 2014; 111(3):1002-7. Epub 2013 Dec. 29. doi: 10.1073/pnas.1313768111. PubMed PMID: 24371310; PMCID: PMC3903238.
10. Castellanos-Rubio A, Fernandez-Jimenez N, Kratchmarov R, Luo X, Bhagat G, Green P H, Schneider R, Kiledjian M, Bilbao J R, Ghosh S. A long noncoding RNA associated with susceptibility to celiac disease. Science. 2016; 352(6281):91-5. Epub 2016 Apr. 2. doi: 10.1126/science.aad0467. PubMed PMID: 27034373; PMCID: PMC4994711.
11. Chan J, Atianand M, Jiang Z, Carpenter S, Aiello D, Elling R, Fitzgerald K A, Caffrey D R. Cutting Edge: A Natural Antisense Transcript, A S-I L 1 alpha, Controls Inducible Transcription of the Proinflammatory Cytokine I L-1 alpha. J Immunol. 2015; 195(4):1359-63. Epub 2015 Jul. 17. doi: 10.4049/jimmunol. 1500264. PubMed PMID: 26179904; PMCID: PMC4530055.
12. Agarwal S, Vierbuchen T, Ghosh S, Chan J, Jiang Z, Kandasamy R K, Ricci E, Fitzgerald K A. The long non-coding RNA LUCAT1 is a negative feedback regulator of interferon responses in humans. Nat Commun. 2020; 11(1):6348. Epub 2020 Dec. 15. doi: 10.1038/s41467-020-20165-5. PubMed PMID: 33311506; PMCID: PMC7733444.
13. Derrien et al., The GENCODE v7 catalog of human long noncoding RNAs: analysis of their gene structure, evolution, and expression. Genome Res. 2012; 22(9):1775-89. Epub 2012 Sep. 8. doi: 10.1101/gr. 132159.111. PubMed PMID: 22955988; PMCID: PMC3431493.
14. Yarani R, Mirza A H, Kaur S, Pociot F. The emerging role of lncRNAs in inflammatory bowel disease. Exp Mol Med. 2018; 50(12):1-14. Epub 2018 Dec. 14. doi: 10.1038/s12276-018-0188-9. PubMed PMID: 30523244; PMCID: PMC6283835.
15. Mirza A H, Berthelsen C H, Seemann S E, Pan X, Frederiksen K S, Vilien M, Gorodkin J, Pociot F. Transcriptomic landscape of lncRNAs in inflammatory bowel disease. Genome Med. 2015; 7(1):39. Epub 2015 May 21. doi: 10.1186/s13073-015-0162-2. PubMed PMID: 25991924; PMCID: PMC4437449.
16. Chen et al., Effect of Long Noncoding RNA H19 Overexpression on Intestinal Barrier Function and Its Potential Role in the Pathogenesis of Ulcerative Colitis. Inflamm Bowel Dis. 2016; 22(11):2582-92. Epub 2016 Oct. 19. doi: 10.1097/MIB.0000000000000932. PubMed PMID: 27661667.
17. Zou T, Jaladanki S K, Liu L, Xiao L, Chung H K, Wang J Y, Xu Y, Gorospe M, Wang J Y. H19 Long Noncoding RNA Regulates Intestinal Epithelial Barrier Function via MicroRNA 675 by Interacting with RNA-Binding Protein HuR. Mol Cell Biol. 2016; 36(9):1332-41. Epub 2016 Feb. 18. doi: 10.1128/MCB.01030-15. PubMed PMID: 26884465; PMCID: PMC4836219.
18. Liu R, Tang A, Wang X, Chen X, Zhao L, Xiao Z, Shen S. Inhibition of lncRNA NEAT1 suppresses the inflammatory response in IBD by modulating the intestinal epithelial barrier and by exosome-mediated polarization of macrophages. Int J Mol Med. 2018; 42(5):2903-13. Epub 2018 Aug. 23. doi: 10.3892/ijmm.2018.3829. PubMed PMID: 30132508.
19. Han J, Li Y, Zhang B, Liu H, Wu M, Zhang X. lncRNA TUG1 regulates ulcerative colitis through miR-142-5p/SOCS1 axis. Microb Pathog. 2020; 143:104139. Epub 2020 Mar. 17. doi: 10.1016/j.micpath.2020.104139. PubMed PMID: 32173492.
20. Wu F, Huang Y, Dong F, Kwon J H. Ulcerative Colitis-Associated Long Noncoding RNA, BC012900, Regulates Intestinal Epithelial Cell Apoptosis. Inflamm Bowel Dis. 2016; 22(4):782-95. Epub 2016 Mar. 5. doi: 10.1097/MIB.0000000000000691. PubMed PMID: 26937624.
21. Ding G, Ming Y, Zhang Y. lncRNA Mirt2 Is Downregulated in Ulcerative Colitis and Regulates IL-22 Expression and Apoptosis in Colonic Epithelial Cells. Gastroenterol Res Pract. 2019; 2019:8154692. Epub 2019 Nov. 7. doi: 10.1155/2019/8154692. PubMed PMID: 31687015; PMCID: PMC6800894.
22. Padua D, Mahurkar-Joshi S, Law I K, Polytarchou C, Vu J P, Pisegna J R, Shih D, Iliopoulos D, Pothoulakis C. A long noncoding RNA signature for ulcerative colitis identifies IFNG-ASI as an enhancer of inflammation. Am J Physiol Gastrointest Liver Physiol. 2016; 311(3):G446-57. Epub 2016 Aug. 6. doi: 10.1152/ajpgi.00212.2016. PubMed PMID: 27492330; PMCID: PMC5076004.
23. Zhu M, Xie J. LncRNA MALATI Promotes Ulcerative Colitis by Upregulating lncRNA ANRIL. Dig Dis Sci. 2020; 65(11):3191-6. Epub 2020 Feb. 7. doi: 10.1007/s10620-020-06093-w. PubMed PMID: 32026279.
24. Gross-Vered M, Shmuel-Galia L, Zarmi B, Humphries F, Thaiss C, Salame™, David E, Chappell-Maor L, Fitzgerald K A, Shai Y, Jung S. TLR2 Dimerization Blockade Allows Generation of Homeostatic Intestinal Macrophages under Acute Colitis Challenge. J Immunol. 2020; 204(3):707-17. Epub 2019 Dec. 29. doi: 10.4049/jimmunol. 1900470. PubMed PMID: 31882517.
25. Deveson I W, Brunck M E, Blackburn J, Tseng E, Hon T, Clark T A, Clark M B, Crawford J, Dinger M E, Nielsen L K, Mattick J S, Mercer T R. Universal Alternative Splicing of Noncoding Exons. Cell Syst. 2018; 6(2):245-55 e5. Epub 2018 Feb. 6. doi: 10.1016/j.cels.2017.12.005. PubMed PMID: 29396323.
26. Argmann et al., Molecular Characterization of Limited Ulcerative Colitis Reveals Novel Biology and Predictors of Disease Extension. Gastroenterology. 2021; 161(6):1953-68 e15. Epub 2021 Sep. 5. doi: 10.1053/j.gastro.2021.08.053. PubMed PMID: 34480882; PMCID: PMC8640960.
27. Suarez-Farinas et al., Intestinal Inflammation Modulates the Expression of ACE2 and TMPRSS2 and Potentially Overlaps With the Pathogenesis of SARS-COV-2-related Disease. Gastroenterology. 2021; 160(1):287-301 e20. Epub 2020 Sep. 28. doi: 10.1053/j.gastro.2020.09.029. PubMed PMID: 32980345; PMCID: PMC7516468.
28. Caffrey D R, Dana P H, Mathur V, Ocano M, Hong E J, Wang Y E, Somaroo S, Caffrey B E, Potluri S, Huang E S. PFAAT version 2.0: a tool for editing, annotating, and analyzing multiple sequence alignments. BMC Bioinformatics. 2007; 8:381. Epub 2007 Oct. 13. doi: 10.1186/1471-2105-8-381. PubMed PMID: 17931421; PMCID: PMC2092438.
29. Cabili M N, Trapnell C, Goff L, Koziol M, Tazon-Vega B, Regev A, Rinn J L. Integrative annotation of human large intergenic noncoding RNAs reveals global properties and specific subclasses. Genes Dev. 2011; 25(18):1915-27. Epub 2011 Sep. 6. doi: 10.1101/gad. 17446611. PubMed PMID: 21890647; PMCID: PMC3185964.
30. Bridges M C, Daulagala A C, Kourtidis A. LNCcation: lncRNA localization and function. J Cell Biol. 2021; 220(2). Epub 2021 Jan. 20. doi: 10.1083/jcb.202009045. PubMed PMID: 33464299; PMCID: PMC7816648.
31. Statello L, Guo C J, Chen L L, Huarte M, Author Correction: Gene regulation by long non-coding RNAs and its biological functions. Nat Rev Mol Cell Biol. 2021; 22(2):159. Epub 2021 Jan. 10. doi: 10.1038/s41580-021-00330-4. PubMed PMID: 33420484; PMCID: PMC8095262.
32. Noh J H, Kim K M, McClusky W G, Abdelmohsen K, Gorospe M. Cytoplasmic functions of long noncoding RNAs. Wiley Interdiscip Rev RNA. 2018; 9(3):e1471. Epub 2018 Mar. 9. doi: 10.1002/wrna. 1471. PubMed PMID: 29516680; PMCID: PMC5963534.
33. Levitt N, Briggs D, Gil A, Proudfoot N J. Definition of an efficient synthetic poly (A) site. Genes Dev. 1989; 3(7):1019-25. Epub 1989 Jul. 1. doi: 10.1101/gad.3.7.1019. PubMed PMID: 2570734.
34. Chu C, Chang H Y. ChIRP-M S: RNA-Directed Proteomic Discovery. Methods Mol Biol. 2018; 1861:37-45. Epub 2018 Sep. 16. doi: 10.1007/978-1-4939-8766-5_3. PubMed PMID: 30218357.
35. Huang J, Nguyen V H, Hamblin K A, Maytum R, van der Giezen M, Fraser M E. ATP-specificity of succinyl-CoA synthetase from Blastocystis hominis. Acta Crystallogr D Struct Biol. 2019; 75 (Pt 7):647-59. Epub 2019 Jul. 10. doi: 10.1107/S2059798319007976. PubMed PMID: 31282474.
36. Noh J H, Kim K M, Abdelmohsen K, Yoon J H, Panda A C, Munk R, Kim J, Curtis J, Moad C A, Wohler C M, Indig F E, de Paula W, Dudekula D B, De S, Piao Y, Yang X, Martindale J L, de Cabo R, Gorospe M. HuR and GRSF1 modulate the nuclear export and mitochondrial localization of the lncRNA RMRP. Genes Dev. 2016; 30(10):1224-39. Epub 2016 May 21. doi: 10.1101/gad.276022.115. PubMed PMID: 27198227; PMCID: PMC4888842.
37. Leucci E, Vendramin R, Spinazzi M, Laurette P, Fiers M, Wouters J, Radaelli E, Eyckerman S, Leonelli C, Vanderheyden K, Rogiers A, Hermans E, Baatsen P, Aerts S, Amant F, Van Aelst S, van den Oord J, de Strooper B, Davidson I, Lafontaine D L, Gevaert K, Vandesompele J, Mestdagh P, Marine J C. Melanoma addiction to the long non-coding RNA SAMMSON. Nature. 2016; 531(7595):518-22. Epub 2016 Mar. 25. doi: 10.1038/nature17161. PubMed PMID: 27008969.
38. Tran K V, Brown E L, DeSouza T, Jespersen N Z, Nandrup-Bus C, Yang Q, Yang Z, Desai A, Min S Y, Rojas-Rodriguez R, Lundh M, Feizi A, Willenbrock H, Larsen T J, Severinsen M C K, Malka K, Mozzicato A M, Deshmukh A S, Emanuelli B, Pedersen B K, Fitzgibbons T, Scheele C, Corvera S, Nielsen S. Human thermogenic adipocyte regulation by the long noncoding RNA LINC00473. Nat Metab. 2020; 2(5):397-412. Epub 2020 May 23. doi: 10.1038/s42255-020-0205-x. PubMed PMID: 32440655; PMCID: PMC7241442.
39. Haberman et al., Ulcerative colitis mucosal transcriptomes reveal mitochondriopathy and personalized mechanisms underlying disease severity and treatment response. Nat Commun. 2019; 10(1):38. Epub 2019 Jan. 4. doi: 10.1038/s41467-018-07841-3. PubMed PMID: 30604764; PMCID: PMC6318335.
40. Noble C L, Abbas A R, Cornelius J, Lees C W, Ho G T, Toy K, Modrusan Z, Pal N, Zhong F, Chalasani S, Clark H, Arnott I D, Penman I D, Satsangi J, Diehl L. Regional variation in gene expression in the healthy colon is dysregulated in ulcerative colitis. Gut. 2008; 57(10):1398-405. Epub 2008 Jun. 5. doi: 10.1136/gut.2008.148395. PubMed PMID: 18523026.
41. Hsieh S Y, Shih T C, Yeh C Y, Lin C J, Chou Y Y, Lee Y S. Comparative proteomic studies on the pathogenesis of human ulcerative colitis. Proteomics. 2006; 6(19):5322-31. Epub 2006 Sep. 2. doi: 10.1002/pmic.200500541. PubMed PMID: 16947118.
42. de Carvalho C, Caramujo M J. The Various Roles of Fatty Acids. Molecules. 2018; 23(10). Epub 2018 Oct. 12. doi: 10.3390/molecules23102583. PubMed PMID: 30304860; PMCID: PMC6222795.
43. Donohoe D R, Garge N, Zhang X, Sun W, O'Connell™, Bunger M K, Bultman S J. The microbiome and butyrate regulate energy metabolism and autophagy in the mammalian colon. Cell Metab. 2011; 13(5):517-26. Epub 2011 May 3. doi: 10.1016/j.cmet.2011.02.018. PubMed PMID: 21531334; PMCID: PMC3099420.
44. Harig J M, Soergel K H, Komorowski R A, Wood C M. Treatment of diversion colitis with short-chain-fatty acid irrigation. N Engl J Med. 1989; 320(1):23-8. Epub 1989 Jan. 5. doi: 10.1056/NEJM198901053200105. PubMed PMID: 2909876.
45. Roediger W E, Nance S. Metabolic induction of experimental ulcerative colitis by inhibition of fatty acid oxidation. Br J Exp Pathol. 1986; 67(6):773-82. Epub 1986 Dec. 1. PubMed PMID: 3099821; PMCID: PMC2013114.
46. Peltekova V D, Wintle R F, Rubin L A, Amos C I, Huang Q, Gu X, Newman B, Van Oene M, Cescon D, Greenberg G, Griffiths A M, St George-Hyslop P H, Siminovitch K A. Functional variants of OCTN cation transporter genes are associated with Crohn disease. Nat Genet. 2004; 36(5):471-5. Epub 2004 Apr. 27. doi: 10.1038/ng1339. PubMed PMID: 15107849.
47. Li N, Ragheb K, Lawler G, Sturgis J, Rajwa B, Melendez J A, Robinson J P. Mitochondrial complex I inhibitor rotenone induces apoptosis through enhancing mitochondrial reactive oxygen species production. J Biol Chem. 2003; 278(10):8516-25. Epub 2002 Dec. 24. doi: 10.1074/jbc.M210432200. PubMed PMID: 12496265.
48. Chen Y, McMillan-Ward E, Kong J, Israels S J, Gibson S B. Mitochondrial electron-transport-chain inhibitors of complexes I and I I induce autophagic cell death mediated by reactive oxygen species. J Cell Sci. 2007; 120 (Pt 23):4155-66. Epub 2007 Nov. 23. doi: 10.1242/jcs.011163. PubMed PMID: 18032788.
49. Gagliardi M, Matarazzo M R. RIP: RNA Immunoprecipitation. Methods Mol Biol. 2016; 1480:73-86. Epub 2016 Sep. 24. doi: 10.1007/978-1-4939-6380-5_7. PubMed PMID: 27659976.
50. Monteiro L B, Davanzo G G, de Aguiar C F, Moraes-Vieira P M M. Using flow cytometry for mitochondrial assays. MethodsX. 2020; 7:100938. Epub 2020 Jun. 20. doi: 10.1016/j.mex.2020.100938. PubMed PMID: 32551241; PMCID: PMC7289760
51. Youle R J, van der Bliek A M. Mitochondrial fission, fusion, and stress. Science. 2012; 337(6098):1062-5. Epub 2012 Sep. 1. doi: 10.1126/science.1219855. PubMed PMID: 22936770; PMCID: PMC4762028.
52. Zhang X, Du L, Zhang W, Yang Y, Zhou Q, Du G. Therapeutic effects of baicalein on rotenone-induced Parkinson's disease through protecting mitochondrial function and biogenesis. Sci Rep. 2017; 7(1):9968. Epub 2017 Sep. 1. doi: 10.1038/s41598-017-07442-y. PubMed PMID: 28855526; PMCID: PMC5577282.
53. Sun M G, Williams J, Munoz-Pinedo C, Perkins G A, Brown J M, Ellisman M H, Green D R, Frey T G. Correlated three-dimensional light and electron microscopy reveals transformation of mitochondria during apoptosis. Nat Cell Biol. 2007; 9(9):1057-65. Epub 2007 Aug. 28. doi: 10.1038/ncb1630. PubMed PMID: 17721514.
54. Smith R A, Murphy M P. Animal and human studies with the mitochondria-targeted antioxidant MitoQ. Ann N Y Acad Sci. 2010; 1201:96-103. Epub 2010 Jul. 24. doi: 10.1111/j.1749-6632.2010.05627.x. PubMed PMID: 20649545.
55. Bar F, Bochmann W, Widok A, von Medem K, Pagel R, Hirose M, Yu X, Kalies K, Konig P, Bohm R, Herdegen T, Reinicke A T, Buning J, Lehnert H, Fellermann K, Ibrahim S, Sina C. Mitochondrial gene polymorphisms that protect mice from colitis. Gastroenterology. 2013; 145(5):1055-63 e3. Epub 2013 Jul. 23. doi: 10.1053/j.gastro.2013.07.015. PubMed PMID: 23872498.
56. Delpre G, Avidor I, Steinherz R, Kadish U, Ben-Bassat M. Ultrastructural abnormalities in endoscopically and histologically normal and involved colon in ulcerative colitis. Am J Gastroenterol. 1989; 84(9):1038-46. Epub 1989 Sep. 1. PubMed PMID: 2773897.
57. Sifroni K G, Damiani C R, Stoffel C, Cardoso M R, Ferreira G K, Jeremias I C, Rezin G T, Scaini G, Schuck P F, Dal-Pizzol F, Streck E L. Mitochondrial respiratory chain in the colonic mucosal of patients with ulcerative colitis. Mol Cell Biochem. 2010; 342(1-2):111-5. Epub 2010 May 5. doi: 10.1007/s11010-010-0474-x. PubMed PMID: 20440543.
58. Santhanam S, Venkatraman A, Ramakrishna B S. Impairment of mitochondrial acetoacetyl COA thiolase activity in the colonic mucosa of patients with ulcerative colitis. Gut. 2007; 56(11):1543-9. Epub 2007 May 8. doi: 10.1136/gut.2006.108449. PubMed PMID: 17483192; PMCID: PMC2095666.
59. Santhanam S, Rajamanickam S, Motamarry A, Ramakrishna B S, Amirtharaj J G, Ramachandran A, Pulimood A, Venkatraman A. Mitochondrial electron transport chain complex dysfunction in the colonic mucosa in ulcerative colitis. Inflamm Bowel Dis. 2012; 18(11):2158-68. Epub 2012 Mar. 1. doi: 10.1002/ibd.22926. PubMed PMID: 22374887.
60. Mills E L, Kelly B, Logan A, Costa A S H, Varma M, Bryant C E, Tourlomousis P, Dabritz J H M, Gottlieb E, Latorre I, Corr S C, McManus G, Ryan D, Jacobs H T, Szibor M, Xavier R J, Braun T, Frezza C, Murphy M P, O'Neill L A. Succinate Dehydrogenase Supports Metabolic Repurposing of Mitochondria to Drive Inflammatory Macrophages. Cell. 2016; 167(2):457-70 e13. Epub 2016 Sep. 27. doi: 10.1016/j.cell.2016.08.064. PubMed PMID: 27667687; PMCID: PMC5863951.
61. Nakahira K, Haspel J A, Rathinam V A, Lee S J, Dolinay T, Lam H C, Englert J A, Rabinovitch M, Cernadas M, Kim H P, Fitzgerald K A, Ryter S W, Choi A M. Autophagy proteins regulate innate immune responses by inhibiting the release of mitochondrial DNA mediated by the NALP3 inflammasome. Nat Immunol. 2011; 12(3):222-30. Epub 2010 Dec. 15. doi: 10.1038/ni.1980. PubMed PMID: 21151103; PMCID: PMC3079381.
62. Ho G T, Aird R E, Liu B, Boyapati R K, Kennedy N A, Dorward D A, Noble C L, Shimizu T, Carter R N, Chew E T S, Morton N M, Rossi A G, Sartor R B, Iredale J P, Satsangi J. MDR1 deficiency impairs mitochondrial homeostasis and promotes intestinal inflammation. Mucosal Immunol. 2018; 11(1):120-30. Epub 2017 Apr. 13. doi: 10.1038/mi.2017.31. PubMed PMID: 28401939; PMCID: PMC5510721.
63. Bolger A M, Lohse M, Usadel B. Trimmomatic: a flexible trimmer for Illumina sequence data. Bioinformatics. 2014; 30(15):2114-20. Epub 2014 Apr. 4. doi: 10.1093/bioinformatics/btu170. PubMed PMID: 24695404; PMCID: PMC4103590.
64. Langmead B, Salzberg S L. Fast gapped-read alignment with Bowtie 2. Nat Methods. 2012; 9(4):357-9. Epub 2012 Mar. 6. doi: 10.1038/nmeth.1923. PubMed PMID: 22388286; PMCID: PMC3322381.
65. Truong D T, Franzosa E A, Tickle T L, Scholz M, Weingart G, Pasolli E, Tett A, Huttenhower C, Segata N. MetaPhlAn2 for enhanced metagenomic taxonomic profiling. Nat Methods. 2015; 12(10):902-3. Epub 2015 Sep. 30. doi: 10.1038/nmeth.3589. PubMed PMID: 26418763.
66. Patro R, Duggal G, Love M I, Irizarry R A, Kingsford C. Salmon provides fast and bias-aware quantification of transcript expression. Nat Methods. 2017; 14(4):417-9. Epub 2017 Mar. 7. doi: 10.1038/nmeth.4197. PubMed PMID: 28263959; PMCID: PMC5600148.
67. Cunningham et al., Ensembl 2022. Nucleic Acids Res. 2022; 50 (D1): D988-D95. Epub 2021 Nov. 19. doi: 10.1093/nar/gkab1049. PubMed PMID: 34791404; PMCID: PMC8728283.
68. Love M I, Huber W, Anders S. Moderated estimation of fold change and dispersion for RNA-seq data with DESeq2. Genome Biol. 2014; 15(12):550. Epub 2014 Dec. 18. doi: 10.1186/s13059-014-0550-8. PubMed PMID: 25516281; PMCID: PMC4302049.
69. Zhu A, Ibrahim J G, Love M I. Heavy-tailed prior distributions for sequence count data: removing the noise and preserving large differences. Bioinformatics. 2019; 35(12):2084-92. Epub 2018 Nov. 6. doi: 10.1093/bioinformatics/bty 895. PubMed PMID: 30395178; PMCID: PMC6581436.
70. Kechin A, Boyarskikh U, Kel A, Filipenko M. cutPrimers: A New Tool for Accurate Cutting of Primers from Reads of Targeted Next Generation Sequencing. J Comput Biol. 2017; 24(11):1138-43. Epub 2017 Jul. 18. doi: 10.1089/cmb.2017.0096. PubMed PMID: 28715235.
71. Love M I, Soneson C, Hickey P F, Johnson L K, Pierce N T, Shepherd L, Morgan M, Patro R. Tximeta: Reference sequence checksums for provenance identification in RNA-seq. PLOS Comput Biol. 2020; 16(2):e1007664. Epub 2020 Feb. 26. doi: 10.1371/journal.pcbi. 1007664. PubMed PMID: 32097405; PMCID: PMC7059966 following competing interests: R P is a co-founder of Ocean Genomics.
72. Hornung V, Ablasser A, Charrel-Dennis M, Bauernfeind F, Horvath G, Caffrey D R, Latz E, Fitzgerald K A. AIM2 recognizes cytosolic dsDNA and forms a caspase-1-activating inflammasome with ASC. Nature. 2009; 458(7237):514-8. Epub 2009 Jan. 23. doi: 10.1038/nature07725. PubMed PMID: 19158675; PMCID: PMC2726264.
73. Bolyen et al., Author Correction: Reproducible, interactive, scalable and extensible microbiome data science using QIIME 2. Nat Biotechnol. 2019; 37(9):1091. Epub 2019 Aug. 11. doi: 10.1038/s41587-019-0252-6. PubMed PMID: 31399723.
74. Shmuel-Galia L, Aychek T, Fink A, Porat Z, Zarmi B, Bernshtein B, Brenner O, Jung S, Shai Y. Neutralization of pro-inflammatory monocytes by targeting TLR2 dimerization ameliorates colitis. EMBO J. 2016; 35(6):685-98. Epub 2016 Feb. 18. doi: 10.15252/embj.201592649. PubMed PMID: 26884587; PMCID: PMC4801944.
75. Shmuel-Galia L, Humphries F, Lei X, Ceglia S, Wilson R, Jiang Z, Ketelut-Carneiro N, Foley S E, Pechhold S, Houghton J, Muneeruddin K, Shaffer S A, McCormick B A, Reboldi A, Ward D, Marshak-Rothstein A, Fitzgerald K A.

Dysbiosis exacerbates colitis by promoting ubiquitination and accumulation of the innate immune adaptor STING in myeloid cells. Immunity. 2021; 54(6):1137-53 e8. Epub 2021 May 30. doi: 10.1016/j.immuni.2021.05.008. PubMed PMID: 34051146; PMCID: PMC8237382.
76. Beghini F, Mclver L J, Blanco-Miguez A, Dubois L, Asnicar F, Maharjan S, Mailyan A, Manghi P, Scholz M, Thomas A M, Valles-Colomer M, Weingart G, Zhang Y, Zolfo M, Huttenhower C, Franzosa E A, Segata N. Integrating taxonomic, functional, and strain-level profiling of diverse microbial communities with bioBakery 3. Elife. 2021; 10. Epub 2021 May 5. doi: 10.7554/eLife.65088. PubMed PMID: 33944776; PMCID: PMC8096432.
77. Mallick et al., Multivariable association discovery in population-scale meta-omics studies. PLOS Comput Biol. 2021; 17(11):e1009442. Epub 2021 Nov. 17. doi: 10.1371/journal.pcbi.1009442. PubMed PMID: 34784344; PMCID: PMC8714082
78. Vierbuchen et al., The lncRNA LUCATI is elevated in inflammatory disease and restrains inflammation by regulating the splicing and stability of NR4A2. Proc Natl Acad Sci USA. 2023; 120(1):e2213715120. Epub 2022 Dec. 29. doi: 10.1073/pnas.2213715120. PubMed PMID: 36577072; PMCID: PMC9910463.
79. Argmann et al., Molecular Characterization of Limited Ulcerative Colitis Reveals Novel Biology and Predictors of Disease Extension. Gastroenterology. 2021; 161(6):1953-68 e15. Epub 2021 Sep. 5. doi: 10.1053/j.gastro.2021.08.053. PubMed PMID: 34480882; PMCID: PMC8640960.
80. Suarez-Farinas et al., Intestinal Inflammation Modulates the Expression of ACE2 and TMPRSS2 and Potentially Overlaps With the Pathogenesis of SARS-COV-2-related Disease. Gastroenterology. 2021; 160(1):287-301 e20. Epub 2020 Sep. 28. doi: 10.1053/j.gastro.2020.09.029. PubMed PMID: 32980345; PMCID: PMC7516468

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of diagnosing inflammatory bowel disease (IBD) in a subject, the method comprising:
providing a sample from the subject;
detecting a level of a HOXA11AS in the sample; and
comparing the level of the HOXA11AS in the sample to a reference level;
wherein presence of a level of HOXA11AS below the reference level indicates that the subject has IBD.

2. A method comprising:
providing a sample from a subject known or suspected to have IBD; and
detecting a level of a HOXA11AS in the sample; and optionally
comparing the level of the HOXA11AS in the sample to a reference level.

3. The method of claim 1, further comprising administering a treatment for IBD to the subject who has a level of HOXA11AS below the reference level.

4. The method of claim 1, wherein the sample comprises nucleic acids or cells from the intestine, optionally from the distal colon, of the subject.

5. The method of claim 4, wherein the sample comprises an intestinal biopsy or cytology brushing.

6. The method of claim 1, wherein the HOXA11AS is at least 80%, 85%, 90%, 95%, or 99% identical to SEQ ID NO:5 or 6.

7. The method of claim 1, wherein detecting a level of HOXA11AS comprises using RT-PCR.

8. A method of treating IBD in a subject, the method comprising administering to the subject a HOXA11AS nucleic acid comprising the isolated nucleic acid of claim 11 to cells in the intestine of the subject, optionally to myeloid cells in the intestine of the subject.

9. The method of claim 8, wherein the HOXA11AS is at least 80%, 85%, 90%, 95%, or 99% identical to SEQ ID NO:5 or 6.

10. The method of claim 8, wherein the HOXA11AS nucleic acid is naked RNA or a viral vector comprising a sequence encoding HOXA11AS.

11. An isolated nucleic acid encoding HOXA11AS, optionally wherein the HOXA11AS is at least 80%, 85%, 90%, 95%, or 99% identical to SEQ ID NO:5 or 6.

12. The isolated nucleic acid of claim 11, which is linked to or comprised within a nanoparticle, e.g., a lipid nanoparticle, optionally wherein the isolated nucleic acid is a HOXA11AS RNA.

13. An expression vector comprising the isolated nucleic acid of claim 11.

14. The expression vector of claim 13, which is a viral vector, optionally an AAV, adenovirus, or lentivirus.

15. The method of claim 1, wherein the IBD is ulcerative colitis (UC).

16.-20. (canceled)
